(19) United States
(12) Patent Application Publication
(10) Pub. No.: US 2004/0235011 A1
(43) Pub. Date: Nov. 25, 2004

(54) Production of multimeric proteins

(76) Inventors: Richard K. Cooper, Baton Rouge, LA (US); William C. Fioretti, Grapevine, TX (US); Gary G. Cadd, Grapevine, TX (US)

Correspondence Address: JOHN S. PRATT, ESQ, KILPATRICK STOCKTON, LLP, 1100 PEACHTREE STREET, ATLANTA, GA, 30309, US

(21) Appl. No.: 10/746,943

(22) Filed: Dec. 24, 2003

Related U.S. Application Data
(63) Continuation-in-part of application No. 10/609,019, filed on Jun. 26, 2003.
(60) Provisional application No. 60/392,415, filed on Jun. 26, 2002.
(60) Provisional application No. 60/441,392, filed on Jan. 21, 2003.
(60) Provisional application No. 60/441,377, filed on Jan. 21, 2003.
(60) Provisional application No. 60/441,502, filed on Jan. 21, 2003.
(60) Provisional application No. 60/441,405, filed on Jan. 21, 2003.
(60) Provisional application No. 60/441,447, filed on Jan. 21, 2003.
(60) Provisional application No. 60/441,381, filed on Jan. 21, 2003.

(51) Int. Cl. C12Q 1/68; A01K 67/00; C07H 21/04; C12P 21/04

(52) U.S. Cl. 435/6; 435/69.7; 435/320.1; 435/325; 530/350; 536/23.2

(57) ABSTRACT

The present invention provides a new, effective and efficient method of producing multimeric proteins in an individual. Multimeric proteins include associated multimeric proteins (two or more associated polypeptides) and multivalent multimeric proteins (a single polypeptide encoded by more than one gene of interest). Expression and/or formation of the multimeric protein in the individual is achieved by administering a polynucleotide cassette containing genes of interest that encode portions of the multimeric protein to the individual. The polynucleotide cassette may additionally contain one or more pro sequences, prepro sequences, cecropin prepro sequences, and/or cleavage site sequences.

### CROSS-REFERENCE TO RELATED APPLICATIONS

The present application is a continuation-in-part of U.S. patent application Ser. No. 10/609,019 filed on Jun. 26, 2003, and claims the priority benefit of U.S. Provisional Patent Application No. 60/441,392 filed Jan. 21, 2003; U.S. Provisional Patent Application No. 60/441,377 filed Jan. 21, 2003; U.S. Provisional Patent Application No. 60/441,502 filed Jan. 21, 2003; U.S. Provisional Patent Application No. 60/441,405 filed Jan. 21, 2003; U.S. Provisional Patent Application No. 60/441,447 filed Jan. 21, 2003; U.S. Provisional Patent Application No. 60/441,381 filed Jan. 21, 2003; and U.S. Provisional Patent Application No. 60/392,415 filed Jun. 26, 2002.

The U.S. Government has certain rights in this invention. The development of this invention was partially funded by the United States Government under a HATCH grant from the United States Department of Agriculture, partially funded by the United States Government with Formula 1433 funds from the United States Department of Agriculture and partially funded by the United States Government under contract DAAD 19-02016 awarded by the Army.

### FIELD OF THE INVENTION

The present invention relates generally to production of multimeric proteins in a transgenic individual, wherein genes encoding the multimeric proteins are operably-linked to signal sequences, or portions of signal sequences.

### BACKGROUND OF THE INVENTION

Methods for producing multimeric proteins in transgenic animals are desirable for a variety of reasons, including the transgenic animal's potential as biological factories to produce multimeric proteins for pharmaceutical, diagnostic and industrial uses. This potential is attractive to the industry due to the inadequate capacity in facilities used for recombinant production of multimeric proteins and the increasing demand by the pharmaceutical industry for use of these facilities. Numerous attempts to produce transgenic animals have met several problems, including low rates of gene incorporation and unstable gene incorporation. Accordingly, improved gene technologies are needed for the development of transgenic animals for the production of multimeric proteins.

Several of the prior art gene delivery technologies employed viruses that are associated with potentially undesirable side effects and safety concerns. The majority of current gene-delivery technologies useful for gene therapy rely on virus-based delivery vectors, such as adeno and adeno-associated viruses, retroviruses, and other viruses, which have been attenuated to no longer replicate. (Kay, M. A., et al. 2001. Nature Medicine 7:3340).

There are multiple problems associated with the use of viral vectors. Firstly, they are not tissue-specific. In fact, a gene therapy trial using adenovirus was recently halted because the vector was present in the patient's sperm (Gene trial to proceed despite fears that therapy could change child's genetic makeup. The New York Times, Dec. 23, 2001). Secondly, viral vectors are likely to be transiently incorporated, which necessitates re-treating a patient at specified time intervals. (Kay, M. A., et al. 2001. Nature Medicine 7:33-40). Thirdly, there is a concern that a viral-based vector could revert to its virulent form and cause disease. Fourthly, viral-based vectors require a dividing cell for stable integration. Fifthly, viral-based vectors indiscriminately integrate into various cells, which can result in undesirable germline integration. Sixthly, the required high titers needed to achieve the desired effect have resulted in the death of one patient and they are believed to be responsible for induction of cancer in a separate study. (Science, News of the Week, Oct. 4, 2002).

Accordingly, what is needed is a new method to produce multimeric proteins in transgenic animals and humans, in which the vector containing those genes does not cause disease or other unwanted side effects. There is also a need for DNA constructs that would be stably incorporated into the tissues and cells of animals and humans, including cells in the resting state that are not replicating. There is a further recognized need in the art for DNA constructs capable of delivering genes to specific tissues and cells of animals and humans and for producing multimeric proteins in those animals and humans.

### SUMMARY OF THE INVENTION

The present invention provides a new, effective and efficient method of producing multimeric proteins in an individual. Multimeric proteins include associated multimeric proteins (two or more associated polypeptides) and multivalent multimeric proteins (a single polypeptide encoded by more than one gene of interest). Expression and/or formation of the multimeric protein in the individual is achieved by administering a polynucleotide cassette containing the genes of interest to the individual. The polynucleotide cassette may additionally contain one or more pro sequences, prepro sequences, cecropin prepro sequences, and/or cleavage site sequences.

This invention provides polynucleotide cassettes containing two or more genes of interest and two or more pro polynucleotide sequences, wherein each gene of interest is operably-linked to a pro nucleotide sequence. Each of the genes of interest encodes a polypeptide that forms a part of the multimeric protein. One discovery of the present invention is the use of pro portions of prepro signal sequences to facilitate appropriate processing, expression, and/or formation of multimeric proteins in an individual. Several examples of prepro polynucleotides from which a pro polynucleotide can be derived or be a part of are a cecropin prepro, lysozyme prepro, ovomucin prepro, ovotransferrin prepro, a signal peptide for tumor necrosis factor receptor (SEQ ID NO:6), a signal peptide encoded by a polynucleotide sequence provided in one of SEQ ID NOs:7-54 and a signal peptide provide in SEQ ID NO:55. The prepro or pro polynucleotide can be a cecropin prepro or pro polynucleotide selected from the group consisting of cecropin A1, cecropin A2, cecropin B, cecropin C, cecropin D, cecropin E and cecropin F. In a preferred embodiment, the pro polynucleotide is a cecropin B pro polynucleotide having a sequence shown in SEQ ID NO:1 or SEQ ID NO:2. A preferred prepro polynucleotide is a cecropin B polynucleotide having a sequence shown in SEQ ID NO:3 or SEQ ID NO:4.

Another discovery of the present invention is that cecropin prepro sequences facilitate appropriate processing, expression, and/or formation of proteins, including multimeric proteins, in an individual. Accordingly, the present invention includes polynucleotide cassettes containing one or more genes of interest operably-linked to a cecropin prepro sequence. In one embodiment, the polynucleotide cassette contains two or more genes of interest operably-linked to a cecropin prepro sequence. Preferred cecropin prepro polynucleotides are provided in SEQ ID NO:3 and SEQ ID NO:4. The present invention also includes polynucleotide cassettes containing two or more genes of interest operably linked to a cecropin prepro polynucleotide, wherein pro sequences are located between the genes of interest.

These polynucleotide cassettes are administered to an individual for expression of polypeptide sequences and the formation of a protein, and more preferably, a multimeric protein. Preferably, the individual is an animal from which the protein can be harvested. Preferred animals are egg-laying or milk-producing animals.

In one embodiment, the egg-laying transgenic animal is an avian. The method of the present invention may be used in avians including Ratites, Psittaciformes, Falconiformes, Piciformes, Strigiformes, Passeriformes, Coraciformes, Ralliformes, Cuculiformes, Columbiformes, Galliformes, Anseriformes, and Herodiones. Preferably, the egg-laying transgenic animal is a poultry bird. More preferably, the bird is a chicken, turkey, duck, goose or quail. Another preferred bird is a ratite, such as, an emu, an ostrich, a rhea, or a cassowary. Other preferred birds are partridge, pheasant, kiwi, parrot, parakeet, macaw, falcon, eagle, hawk, pigeon, cockatoo, song birds, jay bird, blackbird, finch, warbler, canary, toucan, mynah, or sparrow.

In some embodiments, the polynucleotide cassettes are located within transposon-based vectors that allow for incorporation of the cassettes into the DNA of the individual. The transposon-based vectors of the present invention include a transposase, operably-linked to a first promoter, and a coding sequence for a protein or peptide of interest operably-linked to a second promoter, wherein the coding sequence for the protein or peptide of interest and its operably-linked promoter are flanked by transposase insertion sequences recognized by the transposase. The transposon-based vector also includes the following characteristics: a) one or more modified Kozak sequences comprising ACCATG (SEQ ID NO:5) at the 3′ end of the first promoter to enhance expression of the transposase; b) modifications of the codons for the first several N-terminal amino acids of the transposase, wherein the nucleotide at the third base position of each codon is changed to an A or a T without changing the corresponding amino acid; c) addition of one or more stop codons to enhance the termination of transposase synthesis; and/or, d) addition of an effective polyA sequence operably-linked to the transposase to further enhance expression of the transposase gene. In some embodiments, the effective polyA sequence is an avian optimized polyA sequence.

In one embodiment, the transposon-based vector comprises an avian optimized polyA sequence and does not comprise a modified Kozak sequence comprising ACCATG (SEQ ID NO:5). One example of such a transposon-based vector is the pTnMCS vector (SEQ ID NO:56). In another embodiment the transposon-based vector comprises a) one or more modified Kozak sequences comprising ACCATG (SEQ ID NO:5) at the 3′ end of the first promoter to enhance expression of the transposase; b) modifications of the codons for the first several N-terminal amino acids of the transposase, wherein the third base of each codon was changed to an A or a T without changing the corresponding amino acid; c) addition of one or more stop codons to enhance the termination of transposase synthesis; and, d) addition of an effective polyA sequence operably-linked to the transposase to further enhance expression of the transposase gene. One example of such a transposon-based vector is the pTnMod vector (SEQ ID NO:57).

Accordingly, it is an object of the present invention to provide improved methods for the production of multimeric proteins in an individual.

It is another object of the present invention to provide improved methods for the production of multimeric proteins in an egg-laying animal or a milk-producing animal.

It is yet another object of the present invention to provide improved methods for the production of multimeric proteins in a chicken or quail.

Another object of the present invention is to provide a method to produce an egg or milk containing a multimeric protein.

An advantage of the present invention is that multimeric proteins are produced by transgenic animals much more efficiently and economically than prior art methods, thereby providing a means for large scale production of multimeric proteins.

These and other objects, features and advantages of the present invention will become apparent after a review of the following detailed description of the disclosed embodiments and claims.

### BRIEF DESCRIPTION OF THE FIGURES

FIG. 1 depicts schematically a polynucleotide cassette containing two genes of interest operably-linked to two pro polynucleotides, wherein the first pro polynucleotide is a part of a prepro polynucleotide. “Prom” indicates promoter.

FIG. 2 depicts schematically a polynucleotide cassette containing polynucleotides encoding for a heavy chain and a light chain of an antibody. “Oval prom” indicates an ovalbumin promoter. The polynucleotide cassette contains pro and prepro sequences and is flanked by insertion sequences (IS) recognized by a transposase.

FIG. 3 depicts schematically a polynucleotide cassette containing a cecropin prepro sequence operably-linked to two genes of interest. Between the genes of interest resides a cleavage site indicates by “CS.”

FIG. 4 depicts schematically a polynucleotide cassette containing two genes of interest, a promoter (prom), a signal sequence (SS) and a cleavage site (CS). The polynucleotide cassette is flanked by insertion sequences (IS) recognized by a transposase.

FIG. 5 is a picture of a gel showing partially purified egg white derived from a transgenic avian run under reducing and non-reducing conditions.

### DETAILED DESCRIPTION OF THE INVENTION

The present invention provides a new, effective and efficient method of producing multimeric proteins in an individual. Multimeric proteins include associated multimeric proteins (two or more associated polypeptides) and multivalent multimeric proteins (a single polypeptide encoded by more than one gene of interest). Expression and/or formation of the multimeric protein in the individual is achieved by administering a polynucleotide cassette containing the genes of interest to the individual. The polynucleotide cassette may additionally contain one or more pro sequences, prepro sequences, cecropin prepro sequences, and/or cleavage site sequences.

This invention provides polynucleotide cassettes containing two or more genes of interest and two or more pro polynucleotide sequences, wherein each gene of interest is operably-linked to a pro nucleotide sequence. Each of the genes of interest encodes a polypeptide that forms a part of the multimeric protein. These polynucleotide cassettes are administered to an individual for expression of the polypeptide sequences and expression and/or formation of the multimeric protein. Preferably, the individual is an animal from which the multimeric protein can be harvested. Preferred animals are egg-laying or milk-producing animals. In some embodiments, the polynucleotide cassettes are located within transposon-based vectors that allow for incorporation of the cassettes into the DNA of the individual.

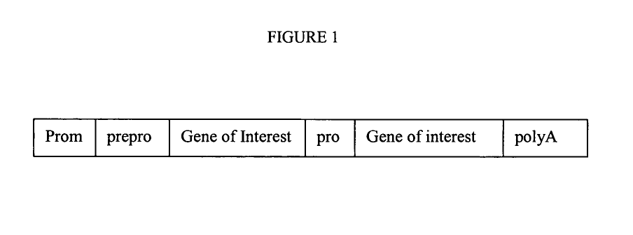

The pro polynucleotide sequences operably-linked to the genes of interest include pro portions of prepro polynucleotide sequences commonly associated with polynucleotides encoding proteins secreted from a cell in nature. It may be that the pre polynucleotide sequence functions to direct the resultant protein into the endoplasmic reticulum and the pro sequence is cleaved within the endoplasmic reticulum or Golgi complex of a cell containing the protein. While prepro polynucleotide sequences are associated with secreted polypeptides in nature, one discovery of the present invention is the use of pro portions of the prepro signal sequences to facilitate appropriate processing, expression, and/or formation of multimeric proteins, and more particularly, associated multimeric proteins. In the present invention, each gene of interest is operably-linked with a pro polynucleotide sequence. FIG. 1 shows schematically one polynucleotide cassette containing two genes of interest, wherein each gene of interest is operably-linked to a pro polynucleotide sequence. The first gene of interest is operably-linked to a pro polynucleotide sequence that is part of a prepro polynucleotide sequence, while the second gene of interest is operably-linked to a pro polynucleotide sequence that is not part of a prepro polynucleotide sequence, but may have been derived from a prepro polynucleotide sequence. Accordingly, the term “pro sequence” encompasses a pro sequence that is part of a prepro sequence and a pro sequence that is not part of a prepro sequence, but may have been derived from a prepro sequence. In preferred embodiments, the most 5′ pro polynucleotide sequence in the polynucleotide cassette is a part of a prepro polynucleotide sequence.

Several examples of prepro polynucleotides from which a pro polynucleotide can be derived or be a part of are a cecropin prepro, lysozyme prepro, ovomucin prepro, ovotransferrin prepro, a signal peptide for tumor necrosis factor receptor (SEQ ID NO:6), a signal peptide encoded by a polynucleotide sequence provided in one of SEQ ID NOs:7-54 and a signal peptide provide in SEQ ID NO:55. The prepro or pro polynucleotide can be a cecropin prepro or pro polynucleotide selected from the group consisting of cecropin A1, cecropin A2, cecropin B, cecropin C, cecropin D, cecropin E and cecropin F. In a preferred embodiment, the pro polynucleotide is a cecropin B pro polynucleotide having a sequence shown in SEQ ID NO:1 or SEQ ID NO:2. A preferred prepro polynucleotide is a cecropin B polynucleotide having a sequence shown in SEQ ID NO:3 or SEQ ID NO:4.

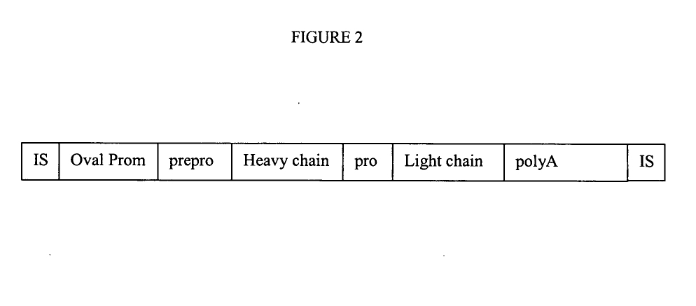

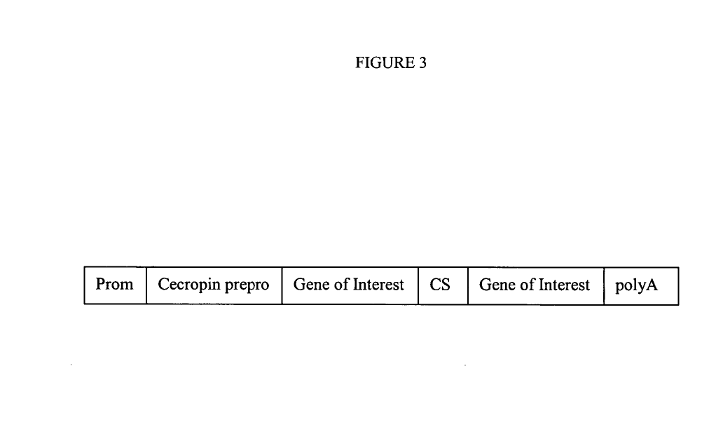

FIG. 1 provides one embodiment of the invention wherein the polynucleotide cassette includes two genes of interest and two pro polynucleotide sequences arranged in the following order: a prepro polynucleotide, a first gene of interest, a pro polynucleotide, and a second gene of interest. Preferably, the sequences are arranged in the aforementioned order beginning at a 5′ end of the polynucleotide cassette. FIG. 2 provides a more specific embodiment of the present invention wherein the first and second genes of interest are polynucleotides encoding antibody heavy and light chains. However, the invention includes polynucleotide cassettes containing at least two genes of interest. Each of the genes of interest is operably-linked to a pro polynucleotide. Each of these pro polynucleotides can be the same, or each can be different. In one embodiment, all of the pro polynucleotides in the polynucleotide cassette are the same and are cecropin pro polynucleotides. The most 5′ cecropin pro polynucleotide is preferably a part of a cecropin prepro polynucleotide sequence as shown in FIG. 3.

The polynucleotide cassettes of the present invention may be administered to an individual for production of a multimeric protein in that individual. Accordingly, the present invention includes a method of producing a multimeric protein in an individual comprising administering to the individual a polynucleotide cassette comprising at least two genes of interest, each encoding a part of the multimeric protein, wherein each gene of interest is operably-linked to a pro polynucleotide sequence. The present invention also includes a method of producing a multimeric protein in an individual comprising administering to the individual a polynucleotide cassette comprising a cecropin prepro sequence operably-linked to two or more genes of interest, each gene of interest encoding a part of the multimeric protein. This second method does not require the linking of pro polynucleotides to each gene of interest since the use of a cecropin prepro sequence itself in a polynucleotide cassette facilitates processing, expression, and/or formation of multimeric proteins. Polynucleotide cassettes containing the cecropin prepro polynucleotide can contain at least two genes of interest. Preferably, the cecropin prepro polynucleotide is located 5′ of the genes of interest in the polynucleotide cassette. One exemplary polynucleotide cassette is shown in FIG. 3. In a preferred embodiment, the prepro sequence comprises a sequence shown in SEQ ID NO:3 or SEQ ID NO:4. As shown in FIG. 3, the polynucleotide cassettes containing a cecropin prepro polynucleotide preferably contain a cleavage site between each of two genes of interest. Such cleavage site(s) may be nucleotides encoding any cleavage sites including, but not limited to, an enzymatic cleavage site, a pro polynucleotide, and a photolabile cleavage site, a chemical cleavage site, and a self-splicing cleavage site (i.e., intein). Cleavage sites are discussed in more detail below.

The polynucleotide cassettes of the present invention are particularly suited for production of multimeric proteins in an individual. Individuals include both humans and animals. Preferred animals are egg-laying animals and milk-producing animals. As used herein, the term “egg-laying animal” includes all amniotes such as birds, turtles, lizards and monotremes. Monotremes are egg-laying mammals and include the platypus and echidna. The term “bird” or “fowl,” as used herein, is defined as a member of the Aves class of animals which are characterized as warm-blooded, egg-laying vertebrates primarily adapted for flying. Avians include, without limitation, Ratites, Psittaciformes, Falconiformes, Piciformes, Strigiformes, Passeriformes, Coraciformes, Ralliformes, Cuculiformes, Columbiformes, Galliformes, Anseriformes, and Herodiones. The term “Ratite,” as used herein, is defined as a group of flightless, mostly large, running birds comprising several orders and including the emus, ostriches, kiwis, and cassowaries. The term “Psittaciformes”, as used herein, includes parrots and refers to a monofamilial order of birds that exhibit zygodactylism and have a strong hooked bill. A “parrot” is defined as any member of the avian family Psittacidae (the single family of the Psittaciformes), distinguished by the short, stout, strongly hooked beak. Preferred avians are poultry birde including chickens, quail, turkeys, geese and ducks. The term “chicken” as used herein denotes chickens used for table egg production, such as egg-type chickens, chickens reared for public meat consumption, or broilers, and chickens reared for both egg and meat production (“dual-purpose” chickens). The term “chicken” also denotes chickens produced by primary breeder companies, or chickens that are the parents, grandparents, great-grandparents, etc. of those chickens reared for public table egg, meat, or table egg and meat consumption.

When the polynucleotide cassettes of the present invention are administered to an egg-laying or milk-producing animal, a transgenic animal containing a polynucleotide cassette is created and the animal produces a transgenic multimeric protein. It is preferred that the resultant multimeric protein is deposited in the egg or in the milk. Various different signal sequences and promoters may be used to achieve deposition of the multimeric protein in the egg or in the milk and these are described in more detail below. In order to achieve a transgenic animal containing a polynucleotide cassette of the present invention, the polynucleotide cassettes can be administered to the individual with, or contained in, any vector, as naked DNA, or in any delivery construct or solution. A preferred vector for incorporation of the polynucleotide cassettes into an individual is a transposon-based vector described below.

Definitions

It is to be understood that as used in the specification and in the claims, “a” or “an” can mean one or more, depending upon the context in which it is used. Thus, for example, reference to “a cell” can mean that at least one cell can be utilized.

The term “antibody” is used interchangeably with the term “immunoglobulin” and is defined herein as a protein synthesized by an animal or a cell of the immune system in response to the presence of a foreign substance commonly referred to as an “antigen” or an “immunogen”. The term antibody includes fragments of antibodies. Antibodies are characterized by specific affinity to a site on the antigen, wherein the site is referred to an “antigenic determinant” or an “epitope”. Antigens can be naturally occurring or artificially engineered. Artificially engineered antigens include but are not limited to small molecules, such as small peptides, attached to haptens such as macromolecules, for example proteins, nucleic acids, or polysaccharides. Artificially designed or engineered variants of naturally occurring antibodies and artificially designed or engineered antibodies not occurring in nature are all included in the current definition. Such variants include conservatively substituted amino acids and other forms of substitution as described in the section concerning proteins and polypeptides.

The term “egg” is defined herein as including a large female sex cell enclosed in a porous, calcarous or leathery shell, produced by birds and reptiles. The term “ovum” is defined as a female gamete, and is also known as an egg. Therefore, egg production in all animals other than birds and reptiles, as used herein, is defined as the production and discharge of an ovum from an ovary, or “ovulation”. Accordingly, it is to be understood that the term “egg” as used herein is defined as a large female sex cell enclosed in a porous, calcarous or leathery shell, when a bird or reptile produces it, or it is an ovum when it is produced by all other animals.

The term “gene” is defined herein to include a polynucleotide that includes a coding region for a protein, peptide or polypeptide, with or without intervening sequences such as introns.

The term “multimeric protein” is defined herein to include one or more polypeptides that are associated, or joined, by any means including disulfde bonds. An example of this type of multimeric protein is an antibody that contains both heavy and light chains that are associated by disulfide bonds. These multimeric proteins are referred to herein as “associated multimeric proteins.” The term “multimeric protein” also includes a polypeptide that is encoded by more than one gene of interest. An example of this type of multimeric protein is a single polypeptide containing a heavy chain polypeptide (first polypeptide of interest) and a light chain polypeptide (second polypeptide of interest). In these embodiments, the different polypeptides of interest may be separated by other polypeptide sequences such as spacer polypeptides and cleavage site polypeptides. These types of multimeric proteins are referred to herein as “multivalent multimeric proteins.”

The term “milk-producing animal” refers herein to mammals including, but not limited to, bovine, ovine, porcine, equine, and primate animals. Milk-producing animals include but are not limited to cows, llamas, camels, goats, reindeer, zebu, water buffalo, yak, horses, pigs, rabbits, non-human primates, and humans.

The term “transgenic animal” refers to an animal having at least a portion of the transposon-based vector DNA is incorporated into its DNA. While a transgenic animal includes an animal wherein the transposon-based vector DNA is incorporated into the germline DNA, a transgenic animal also includes an animal having DNA in one or more somatic cells that contain a portion of the transposon-based vector DNA for any period of time. In a preferred embodiment, a portion of the transposon-based vector comprises a gene of interest. More preferably, the gene of interest is incorporated into the animal's DNA for a period of at least five days, more preferably the laying life of the animal, and most preferably the life of the animal. In a further preferred embodiment, the animal is an avian.

The term “vector” is used interchangeably with the terms “construct”, “DNA construct” and “genetic construct” to denote synthetic nucleotide sequences used for manipulation of genetic material, including but not limited to cloning, subcloning, sequencing, or introduction of exogenous genetic material into cells, tissues or organisms, such as birds. It is understood by one skilled in the art that vectors may contain synthetic DNA sequences, naturally occurring DNA sequences, or both. The vectors of the present invention are transposon-based vectors as described herein.

When referring to two nucleotide sequences, one being a regulatory sequence, the term “operably-linked” is defined herein to mean that the two sequences are associated in a manner that allows the regulatory sequence to affect expression of the other nucleotide sequence. It is not required that the operably-linked sequences be directly adjacent to one another with no intervening sequence(s).

The term “regulatory sequence” is defined herein as including promoters, enhancers and other expression control elements such as polyadenylation sequences, matrix attachment sites, insulator regions for expression of multiple genes on a single construct, ribosome entry/attachment sites, introns that are able to enhance expression, and silencers.

Transposon-Based Vectors

While not wanting to be bound by the following statement, it is believed that the nature of the DNA construct is an important factor in successfully producing transgenic animals. The “standard” types of plasmid and viral vectors that have previously been almost universally used for transgenic work in all species, especially avians, have low efficiencies and may constitute a major reason for the low rates of transformation previously observed. The DNA (or RNA) constructs previously used often do not integrate into the host DNA, or integrate only at low frequencies. Other factors may have also played a part, such as poor entry of the vector into target cells. The present invention provides transposon-based vectors that can be administered to an animal that overcome the prior art problems relating to low transgene integration frequencies. Two preferred transposon-based vectors of the present invention in which a transposase, gene of interest and other polynucleotide sequences may be introduced are termed pTnMCS (SEQ ID NO:56) and pTnMod (SEQ ID NO:57).

The transposon-based vectors of the present invention produce integration frequencies an order of magnitude greater than has been achieved with previous vectors. More specifically, intratesticular injections performed with a prior art transposon-based vector (described in U.S. Pat. No. 5,719,055) resulted in 41% sperm positive roosters whereas intratesticular injections performed with the novel transposon-based vectors of the present invention resulted in 77% sperm positive roosters. Actual frequencies of integration were estimated by either or both comparative strength of the PCR signal from the sperm and histological evaluation of the testes and sperm by quantitative PCR.

The transposon-based vectors of the present invention include a transposase gene operably-linked to a first promoter, and a coding sequence for a desired protein or peptide operably-linked to a second promoter, wherein the coding sequence for the desired protein or peptide and its operably-linked promoter are flanked by transposase insertion sequences recognized by the transposase. The transposon-based vector also includes one or more of the following characteristics: a) one or more modified Kozak sequences comprising ACCATG (SEQ ID NO:5) at the 3′ end of the first promoter to enhance expression of the transposase; b) modifications of one or more of the codons for the first several N-terminal amino acids of the transposase, wherein the third base of each codon was changed to an A or a T without changing the corresponding amino acid; c) addition of one or more stop codons to enhance the termination of transposase synthesis; and/or, d) addition of an effective polyA sequence operably-linked to the transposase to further enhance expression of the transposase gene. In one embodiment, the transposon-based vector comprises an avian optimized polyA sequence and does not comprise a modified Kozak sequence comprising ACCATG (SEQ ID NO:5). One example of such a transposon-based vector is the pTnMCS vector (SEQ ID NO:56). In another embodiment the transposon-based vector comprises a) one or more modified Kozak sequences comprising ACCATG (SEQ ID NO:5) at the 3′ end of the first promoter to enhance expression of the transposase; b) modifications of the codons for the first several N-terminal amino acids of the transposase, wherein the third base of each codon was changed to an A or a T without changing the corresponding amino acid; c) addition of one or more stop codons to enhance the termination of transposase synthesis; and, d) addition of an effective polyA sequence operably-linked to the transposase to further enhance expression of the transposase gene. One example of such a transposon-based vector is the pTnMod vector (SEQ ID NO:57). The transposon-based vector may additionally or alternatively include one or more of the following Kozak sequences at the 3′ end of any promoter, including the promoter operably-linked to the transposase: ACCATGG (SEQ ID NO:58), AAGATGT (SEQ ID NO:59), ACGATGA (SEQ ID NO:60), AAGATGG (SEQ ID NO:61), GACATGA (SEQ ID NO:62), ACCATGA (SEQ ID NO:63), and ACCATGA (SEQ ID NO:64), ACCATGT (SEQ ID NO:65).

Transposases and Insertion Sequences

In a further embodiment of the present invention, the transposase found in the transposase-based vector is an altered target site (ATS) transposase and the insertion sequences are those recognized by the ATS transposase. However, the transposase located in the transposase-based vectors is not limited to a modified ATS transposase and can be derived from any transposase. Transposases known in the prior art include those found in AC7, Tn5SEQ1, Tn916, Tn951, Tn1721, Tn2410, Tn1681, Tn1, Tn2, Tn3, Tn4, Tn5, Tn6, Tn9, Tn10, Tn30, Tn101, Tn903, Tn501, Tn1000 (γδ), Tn1681, Tn2901, AC transposons, Mp transposons, Spm transposons, En transposons, Dotted transposons, Mu transposons, Ds transposons, dSpm transposons and I transposons. According to the present invention, these transposases and their regulatory sequences are modified for improved functioning as follows: a) the addition one or more modified Kozak sequences comprising ACCATG (SEQ ID NO:5) at the 3′ end of the promoter operably-linked to the transposase; b) a change of one or more of the codons for the first several amino acids of the transposase, wherein the third base of each codon was changed to an A or a T without changing the corresponding amino acid; c) the addition of one or more stop codons to enhance the termination of transposase synthesis; and/or, d) the addition of an effective polyA sequence operably-linked to the transposase to further enhance expression of the transposase gene.

Although not wanting to be bound by the following statement, it is believed that the modifications of the first several N-terminal codons of the transposase gene facilitate transcription of the transposase gene, in part, by increasing strand dissociation during transcription. It is preferable that one or more of between approximately the first 1 to 20, more preferably 3 to 15, and most preferably between 4 to 12 N-terminal codons of the transposase are modified such that the third base of each codon is changed to an A or a T without changing the encoded amino acid. In one embodiment, the first ten N-terminal codons of the transposase gene are modified in this manner. It is also preferred that the transposase contain mutations that make it less specific for preferred insertion sites and thus increases the rate of transgene insertion as discussed in U.S. Pat. No. 5,719,055.

In some embodiments, the transposon-based vectors are optimized for expression in a particular host by changing the methylation patterns of the vector DNA. For example, prokaryotic methylation may be reduced by using a methylation deficient organism for production of the transposon-based vector. The transposon-based vectors may also be methylated to resemble eukaryotic DNA for expression in a eukaryotic host.

Transposases and insertion sequences from other analogous eukaryotic transposon-based vectors that can also be modified and used are, for example, the Drosophila P element derived vectors disclosed in U.S. Pat. No. 6,291,243; the Drosophila mariner element described in Sherman et al. (1998); or the sleeping beauty transposon. See also Hackett et al. (1999); D. Lampe et al., 1999. Proc. Natl. Acad. Sci. USA, 96:11428-11433; S. Fischer et al., 2001. Proc. Natl. Acad. Sci. USA, 98:6759-6764; L. Zagoraiou et al., 2001. Proc. Natl. Acad. Sci. USA, 98:11474-11478; and D. Berg et al. (Eds.), Mobile DNA, Amer. Soc. Microbiol. (Washington, D.C., 1989). However, it should be noted that bacterial transposon-based elements are preferred, as there is less likelihood that a eukaryotic transposase in the recipient species will recognize prokaryotic insertion sequences bracketing the transgene.

Many transposases recognize different insertion sequences, and therefore, it is to be understood that a transposase-based vector will contain insertion sequences recognized by the particular transposase also found in the transposase-based vector. In a preferred embodiment of the invention, the insertion sequences have been shortened to about 70 base pairs in length as compared to those found in wild-type transposons that typically contain insertion sequences of well over 100 base pairs.

While the examples provided below incorporate a “cut and insert” Tn10 based vector that is destroyed following the insertion event, the present invention also encompasses the use of a “rolling replication” type transposon-based vector. Use of a rolling replication type transposon allows multiple copies of the transposon/transgene to be made from a single transgene construct and the copies inserted. This type of transposon-based system thereby provides for insertion of multiple copies of a transgene into a single genome. A rolling replication type transposon-based vector may be preferred when the promoter operably-linked to gene of interest is endogenous to the host cell and present in a high copy number or highly expressed. However, use of a rolling replication system may require tight control to limit the insertion events to non-lethal levels. Tn1, Tn2, Tn3, Tn4, Tn5, Tn9, Tn21, Tn501, Tn551, Tn951, Tn1721, Tn2410 and Tn2603 are examples of a rolling replication type transposon, although Tn5 could be both a rolling replication and a cut and insert type transposon.

Stop Codons and PolyA Sequences

In one embodiment, the transposon-based vector contains two stop codons operably-linked to the transposase and/or to the gene of interest. In an alternate embodiment, one stop codon of UAA or UGA is operably linked to the transposase and/or to the gene of interest. While not wanting to be bound by the following statement, it is thought that the stop codon UAG is less effective in translation termination and is therefore less desirable in the constructs described herein.

As used herein an “effective polyA sequence” refers to either a synthetic or non-synthetic sequence that contains multiple and sequential nucleotides containing an adenine base (an A polynucleotide string) and that increases expression of the gene to which it is operably-linked. A polyA sequence may be operably-linked to any gene in the transposon-based vector including, but not limited to, a transposase gene and a gene of interest. A preferred polyA sequence is optimized for use in the host animal or human. In one embodiment, the polyA sequence is optimized for use in an avian species and more specifically, a chicken. An avian optimized polyA sequence generally contains a minimum of 40 base pairs, preferably between approximately 40 and several hundred base pairs, and more preferably approximately 75 base pairs that precede the A polynucleotide string and thereby separate the stop codon from the A polynucleotide string. In one embodiment of the present invention, the polyA sequence comprises a conalbumin polyA sequence as provided in SEQ ID NO:66 and as taken from GenBank accession # Y00407, base pairs 10651-11058. In another embodiment, the polyA sequence comprises a synthetic polynucleotide sequence shown in SEQ ID NO:67. In yet another embodiment, the polyA sequence comprises an avian optimized polyA sequence provided in SEQ ID NO:68. A chicken optimized polyA sequence may also have a reduced amount of CT repeats as compared to a synthetic polyA sequence.

It is a surprising discovery of the present invention that such an avian optimized poly A sequence increases expression of a polynucleotide to which it is operably-linked in an avian as compared to a non-avian optimized polyA sequence. Accordingly, the present invention includes methods of or increasing incorporation of a gene of interest wherein the gene of interest resides in a transposon-based vector containing a transposase gene and wherein the transposase gene is operably linked to an avian optimized polyA sequence. The present invention also includes methods of increasing expression of a gene of interest in an avian that includes administering a gene of interest to the avian, wherein the gene of interest is operably-linked to an avian optimized polyA sequence. An avian optimized polyA nucleotide string is defined herein as a polynucleotide containing an A polynucleotide string and a minimum of 40 base pairs, preferably between approximately 40 and several hundred base pairs, and more preferably approximately 75 base pairs that precede the A polynucleotide string. The present invention further provides transposon-based vectors containing a gene of interest or transposase gene operably linked to an avian optimized polyA sequence.

Promoters and Enhancers

The first promoter operably-linked to the transposase gene and the second promoter operably-linked to the gene of interest can be a constitutive promoter or an inducible promoter. Constitutive promoters include, but are not limited to, immediate early cytomegalovirus (CMV) promoter, herpes simplex virus 1 (HSV1) immediate early promoter, SV40 promoter, lysozyme promoter, early and late CMV promoters, early and late HSV promoters, β-actin promoter, tubulin promoter, Rous-Sarcoma virus (RSV) promoter, and heat-shock protein (HSP) promoter. Inducible promoters include tissue-specific promoters, developmentally-regulated promoters and chemically inducible promoters. Examples of tissue-specific promoters include the glucose 6 phosphate (G6P) promoter, vitellogenin promoter, ovalbumin promoter, ovomucoid promoter, conalbumin promoter, ovotransferrin promoter, prolactin promoter, kidney uromodulin promoter, and placental lactogen promoter. In one embodiment, the vitellogenin promoter includes a polynucleotide sequence of SEQ ID NO:69. The G6P promoter sequence may be deduced from a rat G6P gene untranslated upstream region provided in GenBank accession number U57552.1. Examples of developmentally-regulated promoters include the homeobox promoters and several hormone induced promoters. Examples of chemically inducible promoters include reproductive hormone induced promoters and antibiotic inducible promoters such as the tetracycline inducible promoter and the zinc-inducible metallothionine promoter.

Other inducible promoter systems include the Lac operator repressor system inducible by IPTG (isopropyl beta-D-thiogalactoside) (Cronin, A. et al. 2001. Genes and Development, v. 15), ecdysone-based inducible systems (Hoppe, U. C. et al. 2000. Mol. Ther. 1:159-164); estrogen-based inducible systems (Braselmann, S. et al. 1993. Proc. Natl. Acad. Sci. 90:1657-1661); progesterone-based inducible systems using a chimeric regulator, GLVP, which is a hybrid protein consisting of the GAL4 binding domain and the herpes simplex virus transcriptional activation domain, VP16, and a truncated form of the human progesterone receptor that retains the ability to bind ligand and can be turned on by RU486 (Wang, et al. 1994. Proc. Natl. Acad. Sci. 91:8180-8184); CID-based inducible systems using chemical inducers of dimerization (CIDs) to regulate gene expression, such as a system wherein rapamycin induces dimerization of the cellular proteins FKBP12 and FRAP (Belshaw, P. J. et al. 1996. J. Chem. Biol. 3:731-738; Fan, L. et al. 1999. Hum. Gene Ther. 10:2273-2285; Shariat, S. F. et al. 2001. Cancer Res. 61:2562-2571; Spencer, D. M. 1996. Curr. Biol. 6:839-847). Chemical substances that activate the chemically inducible promoters can be administered to the animal containing the transgene of interest via any method known to those of skill in the art.

Other examples of cell or tissue-specific and constitutive promoters include but are not limited to smooth-muscle SM22 promoter, including chimeric SM22alpha/telokin promoters (Hoggatt A. M. et al., 2002. Circ Res. 91(12):1151-9); ubiquitin C promoter (Biochim Biophys Acta, 2003. Jan. 3; 1625(1):52-63); Hsf2 promoter; murine COMP (cartilage oligomeric matrix protein) promoter; early B cell-specific mb-1 promoter (Sigvardsson M., et al., 2002. Mol. Cell Biol. 22(24):8539-51); prostate specific antigen (PSA) promoter (Yoshimura I. et al., 2002, J. Urol. 168(6):2659-64); exorh promoter and pineal expression-promoting element (Asaoka Y., et al., 2002. Proc. Natl. Acad. Sci. 99(24):15456-61); neural and liver ceramidase gene promoters (Okino N. et al., 2002. Biochem. Biophys. Res. Commun. 299(1):160-6); PSP94 gene promoter/enhancer (Gabril M. Y. et al., 2002. Gene Ther. 9(23):1589-99); promoter of the human FAT/CD36 gene (Kuriki C., et al., 2002. Biol. Pharm. Bull. 25(11):1476-8); VL30 promoter (Staplin W. R. et al., 2002. Blood Oct. 24, 2002); and IL-10 promoter (Brenner S., et al., 2002. J. Biol. Chem. Dec. 18, 2002).

Examples of avian promoters include, but are not limited to, promoters controlling expression of egg white proteins, such as ovalbumin, ovotransferrin (conalbumin), ovomucoid, lysozyme, ovomucin, g2 ovoglobulin, g3 ovoglobulin, ovoflavoprotein, ovostatin (ovomacroglobin), cystatin, avidin, thiamine-binding protein, glutamyl aminopeptidase minor glycoprotein 1, minor glycoprotein 2; and promoters controlling expression of egg-yolk proteins, such as vitellogenin, very low-density lipoproteins, low density lipoprotein, cobalamin-binding protein, riboflavin-binding protein, biotin-binding protein (Awade, 1996. Z. Lebensm. Unters. Forsch. 202:1-14). An advantage of using the vitellogenin promoter is that it is active during the egg-laying stage of an animal's life-cycle, which allows for the production of the protein of interest to be temporally connected to the import of the protein of interest into the egg yolk when the protein of interest is equipped with an appropriate targeting sequence. In some embodiments, the avian promoter is an oviduct-specific promoter. As used herein, the term “oviduct-specific promoter” includes, but is not limited to, ovalbumin, ovotransferrin (conalbumin), ovomucoid, lysozyme, ovomucin, g2 ovoglobulin, g3 ovoglobulin, ovoflavoprotein, and ovostatin (ovomacroglobin) promoters.

Liver-specific promoters of the present invention include, but are not limited to, the following promoters, vitellogenin promoter, G6P promoter, cholesterol-7-alpha-hydroxylase (CYP7A) promoter, phenylalanine hydroxylase (PAH) promoter, protein C gene promoter, insulin-like growth factor I (IGF-I) promoter, bilirubin UDP-glucuronosyltransferase promoter, aldolase B promoter, furin promoter, metallothioneine promoter, albumin promoter, and insulin promoter.

Also included in the present invention are promoters that can be used to target expression of a protein of interest into the milk of a milk-producing animal including, but not limited to, β lactoglobin promoter, whey acidic protein promoter, lactalbumin promoter and casein promoter.

Promoters associated with cells of the immune system may also be used. Acute phase promoters such as interleukin (IL)-1 and IL-2 may be employed. Promoters for heavy and light chain Ig may also be employed. The promoters of the T cell receptor components CD4 and CD8, B cell promoters and the promoters of CR2 (complement receptor type 2) may also be employed. Immune system promoters are preferably used when the desired protein is an antibody protein.

Also included in this invention are modified promoters/enhancers wherein elements of a single promoter are duplicated, modified, or otherwise changed. In one embodiment, a steroid hormone-binding domain of the ovalbumin promoter is moved from about −6.5 kb to within approximately the first 1000 base pairs of the gene of interest. Modifying an existing promoter with promoter/enhancer elements not found naturally in the promoter, as well as building an entirely synthetic promoter, or drawing promoter/enhancer elements from various genes together on a non-natural backbone, are all encompassed by the current invention.

Accordingly, it is to be understood that the promoters contained within the transposon-based vectors of the present invention may be entire promoter sequences or fragments of promoter sequences. For example, in one embodiment, the promoter operably linked to a gene of interest is an approximately 900 base pair fragment of a chicken ovalbumin promoter (SEQ ID NO:70). The constitutive and inducible promoters contained within the transposon-based vectors may also be modified by the addition of one or more modified Kozak sequences of ACCATG (SEQ ID NO:5).

As indicated above, the present invention includes transposon-based vectors containing one or more enhancers. These enhancers may or may not be operably-linked to their native promoter and may be located at any distance from their operably-linked promoter. A promoter operably-linked to an enhancer is referred to herein as an “enhanced promoter.” The enhancers contained within the transposon-based vectors are preferably enhancers found in birds, and more preferably, an ovalbumin enhancer, but are not limited to these types of enhancers. In one embodiment, an approximately 675 base pair enhancer element of an ovalbumin promoter is cloned upstream of an ovalbumin promoter with 300 base pairs of spacer DNA separating the enhancer and promoter. In one embodiment, the enhancer used as a part of the present invention comprises base pairs 1-675 of a Chicken Ovalbumin enhancer from GenBank accession #S82527.1. The polynucleotide sequence of this enhancer is provided in SEQ ID NO:71.

Also included in some of the transposon-based vectors of the present invention are cap sites and fragments of cap sites. In one embodiment, approximately 50 base pairs of a 5′ untranslated region wherein the capsite resides are added on the 3′ end of an enhanced promoter or promoter. An exemplary 5′ untranslated region is provided in SEQ ID NO:72. A putative cap-site residing in this 5′ untranslated region preferably comprises the polynucleotide sequence provided in SEQ ID NO: 73.

In one embodiment of the present invention, the first promoter operably-linked to the transposase gene is a constitutive promoter and the second promoter operably-linked to the gene of interest is a tissue-specific promoter. In the second embodiment, use of the first constitutive promoter allows for constitutive activation of the transposase gene and incorporation of the gene of interest into virtually all cell types, including the germline of the recipient animal. Although the gene of interest is incorporated into the germline generally, the gene of interest is only expressed in a tissue-specific manner. A transposon-based vector having a constitutive promoter operably-linked to the transposase gene can be administered by any route, and in one embodiment, the vector is administered to an ovary or to an artery leading to the ovary. In another embodiment, the vector is administered into the lumen of the oviduct or into an artery supplying the oviduct.

It should be noted that cell- or tissue-specific expression as described herein does not require a complete absence of expression in cells or tissues other than the preferred cell or tissue. Instead, “cell-specific” or “tissue-specific” expression refers to a majority of the expression of a particular gene of interest in the preferred cell or tissue, respectively.

When incorporation of the gene of interest into the germline is not preferred, the first promoter operably-linked to the transposase gene can be a tissue-specific promoter. For example, transfection of a transposon-based vector containing a transposase gene operably-linked to an oviduct specific promoter such as the ovalbumin promoter provides for activation of the transposase gene and incorporation of the gene of interest in the cells of the oviduct but not into the germline and other cells generally. In this embodiment, the second promoter operably-linked to the gene of interest can be a constitutive promoter or an inducible promoter. In a preferred embodiment, both the first promoter and the second promoter are an ovalbumin promoter. In embodiments wherein tissue-specific expression or incorporation is desired, it is preferred that the transposon-based vector is administered directly to the tissue of interest or to an artery leading to the tissue of interest. In a preferred embodiment, the tissue of interest is the oviduct and administration is achieved by direct injection into the lumen of the oviduct or an artery leading to the oviduct. In a further preferred embodiment, administration is achieved by direct injection into the lumen of the magnum or the infundibulum of the oviduct.

Accordingly, cell specific promoters may be used to enhance transcription in selected tissues. In birds, for example, promoters that are found in cells of the fallopian tube, such as ovalbumin, conalbumin, ovomucoid and/or lysozyme, are used in the vectors to ensure transcription of the gene of interest in the epithelial cells and tubular gland cells of the fallopian tube, leading to synthesis of the desired protein encoded by the gene and deposition into the egg white. In mammals, promoters specific for the epithelial cells of the alveoli of the mammary gland, such as prolactin, insulin, beta lactoglobin, whey acidic protein, lactalbumin, casein, and/or placental lactogen, are used in the design of vectors used for transfection of these cells for the production of desired proteins for deposition into the milk. In liver cells, the G6P promoter may be employed to drive transcription of the gene of interest for protein production. Proteins made in the liver of birds may be delivered to the egg yolk.

In order to achieve higher or more efficient expression of the transposase gene, the promoter and other regulatory sequences operably-linked to the transposase gene may be those derived from the host. These host specific regulatory sequences can be tissue specific as described above or can be of a constitutive nature. For example, an avian actin promoter and its associated polyA sequence can be operably-linked to a transposase in a transposase-based vector for transfection into an avian. Examples of other host specific promoters that could be operably-linked to the transposase include the myosin and DNA or RNA polymerase promoters.

Directing Sequences

In some embodiments of the present invention, the gene of interest is operably-linked to a directing sequence or a sequence that provides proper conformation to the desired protein encoded by the gene of interest. As used herein, the term “directing sequence” refers to both signal sequences and targeting sequences. An egg directing sequence includes, but is not limited to, an ovomucoid signal sequence, an ovalbumin signal sequence, a cecropin prepro sequence, and a vitellogenin targeting sequence. The term “signal sequence” refers to an amino acid sequence, or the polynucleotide sequence that encodes the amino acid sequence, a portion or the entirety of which directs the protein to which it is linked to the endoplasmic reticulum in a eukaryote, and more preferably the translocational pores in the endoplasmic reticulum, or the plasma membrane in a prokaryote, or mitochondria, such as for the purpose of gene therapy for mitochondrial diseases. Signal and targeting sequences can be used to direct a desired protein into, for example, the milk, when the transposon-based vectors are administered to a milk-producing animal.

Signal sequences can also be used to direct a desired protein into, for example, a secretory pathway for incorporation into the egg yolk or the egg white, when the transposon-based vectors are administered to a bird or other egg-laying animal. The present invention also includes a gene of interest operably-linked to a second gene containing a signal sequence. An example of such an embodiment is wherein the gene of interest is operably-linked to the ovalbumin gene that contains an ovalbumin signal sequence. Other signal sequences that can be included in the transposon-based vectors include, but are not limited to the ovotransferrin and lysozyme signal sequences. In one embodiment, the signal sequence is an ovalbumin signal sequence including a sequence shown in SEQ ID NO:74. In another embodiment, the signal sequence is a shortened ovalbumin signal sequence including a sequence shown in SEQ ID NO:75 or SEQ ID NO:76.

As also used herein, the term “targeting sequence” refers to an amino acid sequence, or the polynucleotide sequence encoding the amino acid sequence, which amino acid sequence is recognized by a receptor located on the exterior of a cell. Binding of the receptor to the targeting sequence results in uptake of the protein or peptide operably-linked to the targeting sequence by the cell. One example of a targeting sequence is a vitellogenin targeting sequence that is recognized by a vitellogenin receptor (or the low density lipoprotein receptor) on the exterior of an oocyte. In one embodiment, the vitellogenin targeting sequence includes the polynucleotide sequence of SEQ ID NO:77. In another embodiment, the vitellogenin targeting sequence includes all or part of the vitellogenin gene. Other targeting sequences include VLDL and Apo E, which are also capable of binding the vitellogenin receptor. Since the ApoE protein is not endogenously expressed in birds, its presence may be used advantageously to identify birds carrying the transposon-based vectors of the present invention.

Genes of Interest

The genes of interest in the polynucleotide cassette can be any gene, and preferably are genes that encode portions of multimeric proteins. A gene of interest may contain modifications of the codons for the first several N-terminal amino acids of the gene of interest, wherein the third base of each codon is changed to an A or a T without changing the corresponding amino acid. In one embodiment, the genes of interest are antibody genes or portions of antibody genes. FIG. 2 shows a schematic drawing of a polynucleotide cassette containing an antibody heavy chain and an antibody light chain as two genes of interest. Antibodies used in or encoded by the polynucleotide cassettes of the present invention include, but are not limited to, IgG, IgM, IgA, IgD, IgE, IgY, lambda chains, kappa chains, bi-specific antibodies, and fragments thereof; scFv fragments, Fc fragments, and Fab fragments as well as dimeric, trimeric and oligomeric forms of antibody fragments. Desired antibodies include, but are not limited to, naturally occurring antibodies, human antibodies, humanized antibodies, autoantibodies and hybrid antibodies. Genes encoding modified versions of naturally occurring antibodies or fragments thereof and genes encoding artificially designed antibodies or fragments thereof may be incorporated into the transposon-based vectors of the present invention. Desired antibodies also include antibodies with the ability to bind specific ligands, for example, antibodies against proteins associated with cancer-related molecules, such as anti-her 2, or anti-CA125. Accordingly, the present invention encompasses a polynucleotide cassette as described herein containing one or more genes encoding a heavy immunoglobulin (Ig) chain and a light Ig chain.

Antibodies that may be produced using the present invention include, but are not limited to, antibodies for use in cancer immunotherapy against specific antigens, or for providing passive immunity to an animal or a human against an infectious disease or a toxic agent. The antibodies prepared using the methods of the present invention may also be designed to possess specific labels that may be detected through means known to one of ordinary skill in the art. For example, antibodies may be labeled with a fluorescent label attached that may be detected following exposure to specific wavelengths. Such labeled antibodies may be primary antibodies directed to a specific antigen, for example, rhodamine-labeled rabbit anti-growth hormone, or may be labeled secondary antibodies, such as fluorescein-labeled goat-anti chicken IgG. Such labeled antibodies are known to one of ordinary skill in the art. The antibodies may also be designed to possess specific sequences useful for purification through means known to one of ordinary skill in the art. Labels useful for attachment to antibodies are also known to one of ordinary skill in the art. Some of these labels are described in the “Handbook of Fluorescent Probes and Research Products”, ninth edition, Richard P. Haugland (ed) Molecular Probes, Inc. Eugene, Oreg.), which is incorporated herein in its entirety. Antibodies produced with the present invention may be used as laboratory reagents for numerous applications including radioimmunoassay, western blots, dot blots, ELISA, immunoaffinity columns and other procedures requiring antibodies as known to one of ordinary skill in the art. Such antibodies include primary antibodies, secondary antibodies and tertiary antibodies, which may be labeled or unlabeled.

Additional antibodies that may be made with the practice of the present invention include, but are not limited to, primary antibodies, secondary antibodies, designer antibodies, anti-protein antibodies, anti-peptide antibodies, anti-DNA antibodies, anti-RNA antibodies, anti-hormone antibodies, anti-hypophysiotropic peptides, antibodies against non-natural antigens, anti-anterior pituitary hormone antibodies, anti-posterior pituitary hormone antibodies, anti-venom antibodies, anti-tumor marker antibodies, antibodies directed against epitopes associated with infectious disease, including, anti-viral, anti-bacterial, anti-protozoal, anti-fungal, anti-parasitic, anti-receptor, anti-lipid, anti-phospholipid, anti-growth factor, anti-cytokine, anti-monokine, anti-idiotype, and anti-accessory (presentation) protein antibodies. Antibodies made with the present invention, as well as light chains or heavy chains, may also be used to inhibit enzyme activity.

Antibodies that may be produced using the present invention include, but are not limited to, antibodies made against the following proteins: Bovine γ-Globulin, Serum; Bovine IgG, Plasma; Chicken γ-Globulin, Serum; Human γ-Globulin, Serum; Human IgA, Plasma; Human IgA₁, Myeloma; Human IgA₂, Myeloma; Human IgA₂, Plasma; Human IgD, Plasma; Human IgE, Myeloma; Human IgG, Plasma; Human IgG, Fab Fragment, Plasma; Human IgG, F(ab′)₂ Fragment, Plasma; Human IgG, Fc Fragment, Plasma; Human IgG₁, Myeloma; Human IgG₂, Myeloma; Human IgG₃, Myeloma; Human IgG₄, Myeloma; Human IgM, Myeloma; Human IgM, Plasma; Human Immunoglobulin, Light Chain κ, Urine; Human Immunoglobulin, Light Chains κ and λ, Plasma; Mouse γ-Globulin, Serum; Mouse IgG, Serum; Mouse IgM, Myeloma; Rabbit γ-Globulin, Serum; Rabbit IgG, Plasma; and Rat γ-Globulin, Serum. In one embodiment, the transposon-based vector comprises the coding sequence of light and heavy chains of a murine monoclonal antibody that shows specificity for human seminoprotein (GenBank Accession numbers AY129006 and AY129304 for the light and heavy chains, respectively).

A further non-limiting list of antibodies that recognize other antibodies and that may be produced using the present invention is as follows: Anti-Chicken IgG, heavy (H) & light (L) Chain Specific (Sheep); Anti-Goat γ-Globulin (Donkey); Anti-Goat IgG, Fc Fragment Specific (Rabbit); Anti-Guinea Pig γ-Globulin (Goat); Anti-Human Ig, Light Chain, Type κ Specific; Anti-Human Ig, Light Chain, Type λ Specific; Anti-Human IgA, α-Chain Specific (Goat); Anti-Human IgA, Fab Fragment Specific; Anti-Human IgA, Fc Fragment Specific; Anti-Human IgA, Secretory; Anti-Human IgE, ε-Chain Specific (Goat); Anti-Human IgE, Fc Fragment Specific; Anti-Human IgG, Fc Fragment Specific (Goat); Anti-Human IgG, γ-Chain Specific (Goat); Anti-Human IgG, Fc Fragment Specific; Anti-Human IgG, Fd Fragment Specific; Anti-Human IgG, H & L Chain Specific (Goat); Anti-Human IgG₁, Fc Fragment Specific; Anti-Human IgG₂, Fc Fragment Specific; Anti-Human IgG₂, Fd Fragment Specific; Anti-Human IgG₃, Hinge Specific; Anti-Human IgG₄, Fc Fragment Specific; Anti-Human IgM, Fc Fragment Specific; Anti-Human IgM, μ-Chain Specific; Anti-Mouse IgE, ε-Chain Specific; Anti-Mouse γ-Globulin (Goat); Anti-Mouse IgG, γ-Chain Specific (Goat); Anti-Mouse IgG, γ-Chain Specific (Goat) F(ab′)₂ Fragment; Anti-Mouse IgG, H & L Chain Specific (Goat); Anti-Mouse IgM, μ-Chain Specific (Goat); Anti-Mouse IgM, H & L Chain Specific (Goat); Anti-Rabbit γ-Globulin (Goat); Anti-Rabbit IgG, Fc Fragment Specific (Goat); Anti-Rabbit IgG, H & L Chain Specific (Goat); Anti-Rat γ-Globulin (Goat); Anti-Rat IgG, H & L Chain Specific; Anti-Rhesus Monkey γ-Globulin (Goat); and, Anti-Sheep IgG, H & L Chain Specific.

Antibodies that bind a particular ligand may also be produced. Exemplary ligands are as follows: adrenomedulin, amylin, calcitonin, amyloid, calcitonin gene-related peptide, cholecystokinin, gastrin, gastric inhibitory peptide, gastrin releasing peptide, interleukin, interferon, cortistatin, somatostatin, endothelin, sarafotoxin, glucagon, glucagon-like peptide, insulin, atrial natriuretic peptide, BNP, CNP, neurokinin, substance P, leptin, neuropeptide Y, melanin concentrating hormone, melanocyte stimulating hormone, orphanin, endorphin, dynorphin, enkephalin, enkephalin, leumorphin, peptide F, PACAP, PACAP-related peptide, parathyroid hormone, urocortin, corticotrophin releasing hormone, PHM, PHI, vasoactive intestinal polypeptide, secretin, ACTH, angiotensin, angiostatin, bombesin, endostatin, bradykinin, FMRF amide, galanin, gonadotropin releasing hormone (GnRH) associated peptide, GnRH, growth hormone releasing hormone, inhibin, granulocyte-macrophage colony stimulating factor (GM-CSF), motilin, neurotensin, oxytocin, vasopressin, osteocalcin, pancreastatin, pancreatic polypeptide, peptide YY, proopiomelanocortin, transforming growth factor, vascular endothelial growth factor, vesicular monoamine transporter, vesicular acetylcholine transporter, ghrelin, NPW, NPB, C3d, prokinetican, thyroid stimulating hormone, luteinizing hormone, follicle stimulating hormone, prolactin, growth hormone, beta-lipotropin, melatonin, kallikriens, kinins, prostaglandins, erythropoietin, p146 (SEQ ID NO:78 amino acid sequence, SEQ ID NO:79, nucleotide sequence), estrogen, testosterone, corticosteroids, mineralocorticoids, thyroid hormone, thymic hormones, connective tissue proteins, nuclear proteins, actin, avidin, activin, agrin, albumin, and prohormones, propeptides, splice variants, fragments and analogs thereof.

The following is yet another non-limiting of antibodies that can be produced by the methods of present invention: abciximab (ReoPro), abciximab anti-platelet aggregation monoclonal antibody, anti-CD11a (hu1124), anti-CD18 antibody, anti-CD20 antibody, anti-cytomegalovirus (CMV) antibody, anti-digoxin antibody, anti-hepatitis B antibody, anti-HER-2 antibody, anti-idiotype antibody to GD3 glycolipid, anti-IgE antibody, anti-IL-2R antibody, antimetastatic cancer antibody (mAb 17-1A), anti-rabies antibody, anti-respiratory syncytial virus (RSV) antibody, anti-Rh antibody, anti-TCR, anti-TNF antibody, anti-VEGF antibody and Fab fragment thereof, rattlesnake venom antibody, black widow spider venom antibody, coral snake venom antibody, antibody against very late antigen-4 (VLA-4), C225 humanized antibody to EGF receptor, chimeric (human & mouse) antibody against TNFα, antibody directed against GPIIb/IIIa receptor on human platelets, gamma globulin, anti-hepatitis B immunoglobulin, human anti-D immunoglobulin, human antibodies against S aureus, human tetanus immunoglobulin, humanized antibody against the epidermal growth receptor-2, humanized antibody against the α subunit of the interleukin-2 receptor, humanized antibody CTLA41G, humanized antibody to the IL-2 R α-chain, humanized anti-CD40-ligand monoclonal antibody (5c8), humanized mAb against the epidermal growth receptor-2, humanized mAb to rous sarcoma virus, humanized recombinant antibody (IgG1k) against respiratory syncytial virus (RSV), lymphocyte immunoglobulin (anti-thymocyte antibody), lymphocyte immunoglobulin, mAb against factor VII, MDX-210 bi-specific antibody against HER-2, MDX-22, MDX-220 bi-specific antibody against TAG-72 on tumors, MDX-33 antibody to FcγR1 receptor, MDX-447 bi-specific antibody against EGF receptor, MDX-447 bispecific humanized antibody to EGF receptor, MDX-RA immunotoxin (ricin A linked) antibody, Medi-507 antibody (humanized form of BTI-322) against CD2 receptor on T-cells, monoclonal antibody LDP-02, muromonab-CD3(OKT3) antibody, OKT3 (“muromomab-CD3”) antibody, PRO542 antibody, ReoPro (“abciximab”) antibody, and TNF-IgG fusion protein.

Another non-limiting list of the antibodies that may be produced using the present invention is provided in product catalogs of companies such as Phoenix Pharmaceuticals, Inc. (www.phoenixpeptide.com; 530 Harbor Boulevard, Belmont, Calif.), Peninsula Labs San Carlos Calif., SIGMA, St. Louis, Mo. www.sigma-aldrich.com, Cappel ICN, Irvine, Calif., www.icnbiomed.com, and Calbiochem, La Jolla, Calif., www.calbiochem.com, which are all incorporated herein by reference in their entirety. The polynucleotide sequences encoding these antibodies may be obtained from the scientific literature, from patents, and from databases such as GenBank. Alternatively, one of ordinary skill in the art may design the antibody polynucleotide sequence by choosing the codons that encode for each amino acid in the desired antibody.

Genes encoding protein and peptide hormones are a preferred class of genes of interest in the present invention. Such protein and peptide hormones are synthesized throughout the endocrine system and include, but are not limited to, hypothalamic hormones and hypophysiotropic hormones, anterior, intermediate and posterior pituitary hormones, pancreatic islet hormones, hormones made in the gastrointestinal system, renal hormones, thymic hormones, parathyroid hormones, adrenal cortical and medullary hormones. Specifically, hormones that can be produced using the present invention include, but are not limited to, chorionic gonadotropin, corticotropin, erythropoietin, glucagons, IGF-1, oxytocin, platelet-derived growth factor, calcitonin, follicle-stimulating hormone, luteinizing hormone, thyroid-stimulating hormone, insulin, gonadotropin-releasing hormone and its analogs, vasopressin, octreotide, somatostatin, prolactin, adrenocorticotropic hormone, antidiuretic hormone, thyrotropin-releasing hormone (TRH), growth hormone-releasing hormone (GHRH), dopamine, melatonin, thyroxin (T₄), parathyroid hormone (PTH), glucocorticoids such as cortisol, mineralocorticoids such as aldosterone, androgens such as testosterone, adrenaline (epinephrine), noradrenaline (norepinephrine), estrogens such as estradiol, progesterone, glucagons, calcitrol, calciferol, atrial-natriuretic peptide, gastrin, secretin, cholecystokinin (CCK), neuropeptide Y, ghrelin, PYY₃₋₃₆, angiotensinogen, thrombopoietin, and leptin. By using appropriate polynucleotide sequences, species-specific hormones may be made by transgenic animals.

In one embodiment of the present invention, the gene of interest is a proinsulin gene and the desired molecule is insulin. Proinsulin consists of three parts: a C-peptide and two strands of amino acids (the alpha and beta chains) that later become linked together to form the insulin molecule. In these embodiments, proinsulin is expressed in the oviduct tubular gland cells and then deposited in the egg white. One example of a proinsulin polynucleotide sequence is shown in SEQ ID NO:80, wherein the C-peptide cleavage site spans from Arg at position 31 to Arg at position 65.

Further included in the present invention are genes of interet that encode proteins and peptides synthesized by the immune system including those synthesized by the thymus, lymph nodes, spleen, and the gastrointestinal associated lymph tissues (GALT) system. The immune system proteins and peptides proteins that can be made in transgenic animals using the polynucleotide cassettes of the present invention include, but are not limited to, alpha-interferon, beta-interferon, gamma-interferon, alpha-interferon A, alpha-interferon 1, G-CSF, GM-CSF, interlukin-1 (IL-1), IL-2, IL-3, IL-4, IL-5, IL-6, IL-7, IL-8, IL-9, IL-10, IL-11, IL-12, IL-13, TNF-α, and TNF-β. Other cytokines included in the present invention include cardiotrophin, stromal cell derived factor, macrophage derived chemokine (MDC), melanoma growth stimulatory activity (MGSA), macrophage inflammatory proteins 1 alpha (MIP-1 alpha), 2, 3 alpha, 3 beta, 4 and 5.

Genes encoding lytic peptides such as p146 are also included in the genes of interest of the present invention. In one embodiment, the p146 peptide comprises an amino acid sequence of SEQ ID NO:78. The present invention also encompasses a polynucleotide cassette comprising a p146 nucleic acid having a sequence of SEQ ID NO:79.

Enzymes are another class of proteins that may be encoded by the polynucleotide cassettes of the present invention. Such enzymes include but are not limited to adenosine deaminase, alpha-galactosidase, cellulase, collagenase, dnasel, hyaluronidase, lactase, L-asparaginase, pancreatin, papain, streptokinase B, subtilisin, superoxide dismutase, thrombin, trypsin, urokinase, fibrinolysin, glucocerebrosidase and plasminogen activator. In some embodiments wherein the enzyme could have deleterious effects, additional amino acids and a protease cleavage site are added to the carboxy end of the enzyme of interest in order to prevent expression of a functional enzyme. Subsequent digestion of the enzyme with a protease results in activation of the enzyme.

Extracellular matrix proteins are one class of desired proteins that may be encoded by the polynucleotide cassettes of the present invention. Examples include but are not limited to collagen, fibrin, elastin, laminin, and fibronectin and subtypes thereof. Intracellular proteins and structural proteins are other classes of desired proteins in the present invention.

Growth factors are another desired class of proteins that may be encoded by the polynucleotide cassettes of the present invention and include, but are not limited to, transforming growth factor-α (“TGF-α”), transforming growth factor-β (TGF-β), platelet-derived growth factors (PDGF), fibroblast growth factors (FGF), including FGF acidic isoforms 1 and 2, FGF basic form 2 and FGF 4, 8, 9 and 10, nerve growth factors (NGF) including NGF 2.5s, NGF 7.0s and beta NGF and neurotrophins, brain derived neurotrophic factor, cartilage derived factor, growth factors for stimulation of the production of red blood cells, growth factors for stimulation of the production of white blood cells, bone growth factors (BGF), basic fibroblast growth factor, vascular endothelial growth factor (VEGF), granulocyte colony stimulating factor (G-CSF), insulin like growth factor (IGF) I and II, hepatocyte growth factor, glial neurotrophic growth factor (GDNF), stem cell factor (SCF), keratinocyte growth factor (KGF), transforming growth factors (TGF), including TGFs alpha, beta, beta1, beta2, beta3, skeletal growth factor, bone matrix derived growth factors, bone derived growth factors, erythropoietin (EPO) and mixtures thereof.

Another desired class of proteins that may be encoded by the polynucleotide cassettes of the present invention include, but are not limited to, leptin, leukemia inhibitory factor (LIF), tumor necrosis factor alpha and beta, ENBREL, angiostatin, endostatin, thrombospondin, osteogenic protein-1, bone morphogenetic proteins 2 and 7, osteonectin, somatomedin-like peptide, and osteocalcin.

Yet another desired class of proteins encoded by the genes of interet are blood proteins or clotting cascade protein including albumin, Prekallikrein, High molecular weight kininogen (HMWK) (contact activation cofactor; Fitzgerald, Flaujeac Williams factor), Factor I (Fibrinogen), Factor II (prothrombin), Factor III (Tissue Factor), Factor IV (calcium), Factor V (proaccelerin, labile factor, accelerator (Ac-) globulin), Factor VI (Va) (accelerin), Factor VII (proconvertin), serum prothrombin conversion accelerator (SPCA), cothromboplastin), Factor VIII (antihemophiliac factor A, antihemophilic globulin (AHG)), Factor IX (Christmas Factor, antihemophilic factor B, plasma thromboplastin component (PTC)), Factor X (Stuart-Prower Factor), Factor XI (Plasma thromboplastin antecedent (PTA)), Factor XII (Hageman Factor), Factor XIII (rotransglutaminase, fibrin stabilizing factor (FSF), fibrinoligase), von Willebrand factor, Protein C, Protein S, Thrombomodulin, Antithrombin III.

A non-limiting list of the peptides and proteins that may be encoded by the polynucleotide cassettes of the present invention is provided in product catalogs of companies such as Phoenix Pharmaceuticals, Inc. (www.phoenixpeptide.com; 530 Harbor Boulevard, Belmont, Calif.), Peninsula Labs (San Carlos Calif.), SIGMA, (St. Louis, Mo. www.sigma-aldrich.com), Cappel ICN (Irvine, Calif., www.icnbiomed.com), and Calbiochem (La Jolla, Calif., www.calbiochem.com). The polynucleotide sequences encoding these proteins and peptides of interest may be obtained from the scientific literature, from patents, and from databases such as GenBank. Alternatively, one of ordinary skill in the art may design the polynucleotide sequence to be incorporated into the genome by choosing the codons that encode for each amino acid in the desired protein or peptide.

Some of these desired proteins or peptides that may be encoded by the polynucleotide cassettes of the present invention include but are not limited to the following: adrenomedulin, amylin, calcitonin, amyloid, calcitonin gene-related peptide, cholecystokinin, gastrin, gastric inhibitory peptide, gastrin releasing peptide, interleukin, interferon, cortistatin, somatostatin, endothelin, sarafotoxin, glucagon, glucagon-like peptide, insulin, atrial natriuretic peptide, BNP, CNP, neurokinin, substance P, leptin, neuropeptide Y, melanin concentrating hormone, melanocyte stimulating hormone, orphanin, endorphin, dynorphin, enkephalin, leumorphin, peptide F, PACAP, PACAP-related peptide, parathyroid hormone, urocortin, corticotrophin releasing hormone, PHM, PHI, vasoactive intestinal polypeptide, secretin, ACTH, angiotensin, angiostatin, bombesin, endostatin, bradykinin, FMRF amide, galanin, gonadotropin releasing hormone (GnRH) associated peptide, GnRH, growth hormone releasing hormone, inhibin, granulocyte-macrophage colony stimulating factor (GM-CSF), motilin, neurotensin, oxytocin, vasopressin, osteocalcin, pancreastatin, pancreatic polypeptide, peptide YY, proopiomelanocortin, transforming growth factor, vascular endothelial growth factor, vesicular monoamine transporter, vesicular acetylcholine transporter, ghrelin, NPW, NPB, C3d, prokinetican, thyroid stimulating hormone, luteinizing hormone, follicle stimulating hormone, prolactin, growth hormone, beta-lipotropin, melatonin, kallikriens, kinins, prostaglandins, erythropoietin, p146 (SEQ ID NO:78, amino acid sequence, SEQ ID NO:79, nucleotide sequence), thymic hormones, connective tissue proteins, nuclear proteins, actin, avidin, activin, agrin, albumin, apolipoproteins, apolipoprotein A, apolipoprotein B, and prohormones, propeptides, splice variants, fragments and analogs thereof.

Other desired proteins that may be encoded by the polynucleotide cassettes of the present invention include bacitracin, polymixin b, vancomycin, cyclosporine, anti-RSV antibody, alpha-1 antitrypsin (AAT), anti-cytomegalovirus antibody, anti-hepatitis antibody, anti-inhibitor coagulant complex, anti-rabies antibody, anti-Rh(D) antibody, adenosine deaminase, anti-digoxin antibody, antivenin crotalidae (rattlesnake venom antibody), antivenin latrodectus (black widow spider venom antibody), antivenin micrurus (coral snake venom antibody), aprotinin, corticotropin (ACTH), diphtheria antitoxin, lymphocyte immune globulin (anti-thymocyte antibody), protamine, thyrotropin, capreomycin, α-galactosidase, gramicidin, streptokinase, tetanus toxoid, tyrothricin, IGF-1, proteins of varicella vaccine, anti-TNF antibody, anti-IL-2r antibody, anti-HER-2 antibody, OKT3 (“muromonab-CD3”) antibody, TNF-IgG fusion protein, ReoPro (“abciximab”) antibody, ACTH fragment 1-24, desmopressin, gonadotropin-releasing hormone, histrelin, leuprolide, lypressin, nafarelin, peptide that binds GPIIb/GPIIIa on platelets (integrilin), goserelin, capreomycin, colistin, anti-respiratory syncytial virus, lymphocyte immune globulin (Thymoglovin, Atgam), panorex, alpha-antitrypsin, botulinin, lung surfactant protein, tumor necrosis receptor-IgG fusion protein (enbrel), gonadorelin, proteins of influenza vaccine, proteins of rotavirus vaccine, proteins of haemophilus b conjugate vaccine, proteins of poliovirus vaccine, proteins of pneumococcal conjugate vaccine, proteins of meningococcal C vaccine, proteins of influenza vaccine, megakaryocyte growth and development factor (MGDF), neuroimmunophilin ligand-A (NIL-A), brain-derived neurotrophic factor (BDNF), glial cell line-derived neurotrophic factor (GDNF), leptin (native), leptin B, leptin C, IL-IRA (interleukin-IRA), R-568, novel erythropoiesis-stimulating protein (NESP), humanized mAb to rous sarcoma virus (MEDI-493), glutamyl-tryptophan dipeptide IM862, LFA-3TIP immunosuppressive, humanized anti-CD40-ligand monoclonal antibody (5c8), gelsonin enzyme, tissue factor pathway inhibitor (TFPI), proteins of meningitis B vaccine, antimetastatic cancer antibody (mAb 17-1A), chimeric (human & mouse) mAb against TNFα, mAb against factor VII, relaxin, capreomycin, glycopeptide (LY333328), recombinant human activated protein C (rhAPC), humanized mAb against the epidermal growth receptor-2, altepase, anti-CD20 antigen, C2B8 antibody, insulin-like growth factor-1, atrial natriuretic peptide (anaritide), tenectaplase, anti-CD11a antibody (hu 1124), anti-CD18 antibody, mAb LDP-02, anti-VEGF antibody, Fab fragment of anti-VEGF Ab, APO2 ligand (tumor necrosis factor-related apoptosis-inducing ligand), rTGF-β (transforming growth factor-β), alpha-antitrypsin, ananain (a pineapple enzyme), humanized mAb CTLA41G, PRO542 (mAb), D2E7 (mAb), calf intestine alkaline phosphatase, α-L-iduronidase, α-L-galactosidase (humanglutamic acid decarboxylase, acid sphingomyelinase, bone morphogenetic protein-2 (rhBMP-2), proteins of HIV vaccine, T cell receptor (TCR) peptide vaccine, TCR peptides, V beta 3 and V beta 13.1. (IR502), (IR501), BI 1050/1272 mAb against very late antigen-4 (VLA-4), C225 humanized mAb to EGF receptor, anti-idiotype antibody to GD3 glycolipid, antibacterial peptide against H. pylori, MDX-447 bispecific humanized mAb to EGF receptor, anti-cytomegalovirus (CMV), Medi-491 B19 parvovirus vaccine, humanized recombinant mAb (IgG1k) against respiratory syncytial virus (RSV), urinary tract infection vaccine (against “pili” on Escherechia coli strains), proteins of lyme disease vaccine against B. burgdorferi protein (DbpA), proteins of Medi-501 human papilloma virus-11 vaccine (HPV), Streptococcus pneumoniae vaccine, Medi-507 mAb (humanized form of BTI-322) against CD2 receptor on T-cells, MDX-33 mAb to FcγR1 receptor, MDX-RA immunotoxin (ricin A linked) mAb, MDX-210 bi-specific mAb against HER-2, MDX-447 bi-specific mAb against EGF receptor, MDX-22, MDX-220 bi-specific mAb against TAG-72 on tumors, colony-stimulating factor (CSF) (molgramostim), humanized mAb to the IL-2 R α-chain (basiliximab), mAb to IgE (IGE 025A), myelin basic protein-altered peptide (MSP771A), humanized mAb against the epidermal growth receptor-2, humanized mAb against the α subunit of the interleukin-2 receptor, low molecular weight heparin, anti-hemophillic factor, and bactericidal/permeability-increasing protein (r-BPI).

Other multimeric proteins that may be produced using the present invention are as follows: factors involved in the synthesis or replication of DNA, such as DNA polymerase alpha and DNA polymerase delta; proteins involved in the production of mRNA, such as TFIID and TFIIH; cell, nuclear and other membrane-associated proteins, such as hormone and other signal transduction receptors, active transport proteins and ion channels, multimeric proteins in the blood, including hemoglobin, fibrinogen and von Willabrand's Factor; proteins that form structures within the cell, such as actin, myosin, and tubulin and other cytoskeletal proteins; proteins that form structures in the extra cellular environment, such as collagen, elastin and fibronectin; proteins involved in intra- and extra-cellular transport, such as kinesin and dynein, the SNARE family of proteins (soluble NSF attachment protein receptor) and clathrin; proteins that help regulate chromatin structure, such as histones and protamines, Swi3p, Rsc8p and moira; multimeric transcription factors such as Fos, Jun and CBTF (CCAAT box transcription factor); multimeric enzymes such as acetylcholinesterase and alcohol dehydrogenase; chaperone proteins such as GroE, Gro EL (chaperonin 60) and Gro ES(chaperonin 10); anti-toxins, such as snake venom, botulism toxin, Streptococcus super antigens; lysins (enzymes from bacteriophage and viruses); as well as most allosteric proteins.

The multimeric proteins made using the present invention may be labeled using labels and techniques known to one of ordinary skill in the art. Some of these labels are described in the “Handbook of Fluorescent Probes and Research Products”, ninth edition, Richard P. Haugland (ed) Molecular Probes, Inc. Eugene, Oreg.), which is incorporated herein in its entirety. Some of these labels may be genetically engineered into the polynucleotide sequence for the expression of the selected multimeric protein. The peptides and proteins may also have label-incorporation “handles” incorporated to allow labeling of an otherwise difficult or impossible to label multimeric protein.

It is to be understood that the various classes of desired peptides and proteins, as well as specific peptides and proteins described in this section may be modified as described below by inserting selected codons for desired amino acid substitutions into the gene incorporated into the transgenic animal.

The present invention may also be used to produce desired molecules other than proteins and peptides including, but not limited to, lipoproteins such as high density lipoprotein (HDL), HDL-Milano, and low density lipoprotein, lipids, carbohydrates, siRNA and ribozymes. In these embodiments, a gene of interest encodes a nucleic acid molecule or a protein that directs production of the desired molecule.

The present invention further encompasses the use of inhibitory molecules to inhibit endogenous (i.e., non-vector) protein production. These inhibitory molecules include antisense nucleic acids, siRNA and inhibitory proteins. In a preferred embodiment, the endogenous protein whose expression is inhibited is an egg white protein including, but not limited to ovalbumin, ovotransferrin, and ovomucin ovomucoid, ovoinhibitor, cystatin, ovostatin, lysozyme, ovoglobulin G2, ovoglobulin G3, avidin, and thiamin binding protein. In one embodiment, a polynucleotide cassette containing an ovalbumin DNA sequence, that upon transcription forms a double stranded RNA molecule, is transfected into an animal such as a bird and the bird's production of endogenous ovalbumin protein is reduced by the interference RNA mechanism (RNAi). In other embodiments, a polynucleotide cassette encodes an inhibitory RNA molecule that inhibits the expression of more than one egg white protein. Additionally, inducible knockouts or knockdowns of the endogenous protein may be created to achieve a reduction or inhibition of endogenous protein production. Endogenous egg white production can be inhibited in an avian at any time, but is preferably inhibited preceding, or immediately preceding, the harvest of eggs.

Modified Desired Proteins and Peptides

The present invention may be used for the production of multimeric proteins. “Proteins”, “peptides,” “polypeptides” and “oligopeptides” are chains of amino acids (typically L-amino acids) whose alpha carbons are linked through peptide bonds formed by a condensation reaction between the carboxyl group of the alpha carbon of one amino acid and the amino group of the alpha carbon of another amino acid. The terminal amino acid at one end of the chain (i.e., the amino terminal) has a free amino group, while the terminal amino acid at the other end of the chain (i.e., the carboxy terminal) has a free carboxyl group. As such, the term “amino terminus” (abbreviated N-terminus) refers to the free alpha-amino group on the amino acid at the amino terminal of the protein, or to the alpha-amino group (imino group when participating in a peptide bond) of an amino acid at any other location within the protein. Similarly, the term “carboxy terminus” (abbreviated C-terminus) refers to the free carboxyl group on the amino acid at the carboxy terminus of a protein, or to the carboxyl group of an amino acid at any other location within the protein.

Typically, the amino acids making up a protein are numbered in order, starting at the amino terminal and increasing in the direction toward the carboxy terminal of the protein. Thus, when one amino acid is said to “follow” another, that amino acid is positioned closer to the carboxy terminal of the protein than the preceding amino acid.

The term “residue” is used herein to refer to an amino acid (D or L) or an amino acid mimetic that is incorporated into a protein by an amide bond. As such, the amino acid may be a naturally occurring amino acid or, unless otherwise limited, may encompass known analogs of natural amino acids that function in a manner similar to the naturally occurring amino acids (i.e., amino acid mimetics). Moreover, an amide bond mimetic includes peptide backbone modifications well known to those skilled in the art.

Furthermore, one of skill will recognize that, as mentioned above, individual substitutions, deletions or additions which alter, add or delete a single amino acid or a small percentage of amino acids (typically less than about 5%, more typically less than about 1%) in an encoded sequence are conservatively modified variations where the alterations result in the substitution of an amino acid with a chemically similar amino acid. Conservative substitution tables providing functionally similar amino acids are well known in the art. The following six groups each contain amino acids that are conservative substitutions for one another:

1) Alanine (A), Serine (S), Threonine (T);

2) Aspartic acid (D), Glutamic acid (E);

3) Asparagine (N), Glutamine (Q);

4) Arginine (R), Lysine (K);

5) Isoleucine (I), Leucine (L), Methionine (M), Valine (V); and

6) Phenylalanine (F), Tyrosine (Y), Tryptophan (W).

A conservative substitution is a substitution in which the substituting amino acid (naturally occurring or modified) is structurally related to the amino acid being substituted, i.e., has about the same size and electronic properties as the amino acid being substituted. Thus, the substituting amino acid would have the same or a similar functional group in the side chain as the original amino acid. A “conservative substitution” also refers to utilizing a substituting amino acid which is identical to the amino acid being substituted except that a functional group in the side chain is protected with a suitable protecting group.

Suitable protecting groups are described in Green and Wuts, “Protecting Groups in Organic Synthesis”, John Wiley and Sons, Chapters 5 and 7, 1991, the teachings of which are incorporated herein by reference. Preferred protecting groups are those which facilitate transport of the peptide through membranes, for example, by reducing the hydrophilicity and increasing the lipophilicity of the peptide, and which can be cleaved, either by hydrolysis or enzymatically (Ditter et al., 1968. J. Pharm. Sci. 57:783; Ditter et al., 1968. J. Pharm. Sci. 57:828; Ditter et al., 1969. J. Pharm. Sci. 58:557; King et al., 1987. Biochemistry 26:2294; Lindberg et al., 1989. Drug Metabolism and Disposition 17:311; Tunek et al., 1988. Biochem. Pharm. 37:3867; Anderson et al., 1985 Arch. Biochem. Biophys. 239:538; and Singhal et al., 1987. FASEB J. 1:220). Suitable hydroxyl protecting groups include ester, carbonate and carbamate protecting groups. Suitable amine protecting groups include acyl groups and alkoxy or aryloxy carbonyl groups, as described above for N-terminal protecting groups. Suitable carboxylic acid protecting groups include aliphatic, benzyl and aryl esters, as described below for C-terminal protecting groups. In one embodiment, the carboxylic acid group in the side chain of one or more glutamic acid or aspartic acid residues in a peptide of the present invention is protected, preferably as a methyl, ethyl, benzyl or substituted benzyl ester, more preferably as a benzyl ester.

Provided below are groups of naturally occurring and modified amino acids in which each amino acid in a group has similar electronic and steric properties. Thus, a conservative substitution can be made by substituting an amino acid with another amino acid from the same group. It is to be understood that these groups are non-limiting, i.e. that there are additional modified amino acids which could be included in each group.

Group I includes leucine, isoleucine, valine, methionine and modified amino acids having the following side chains: ethyl, n-propyl n-butyl. Preferably, Group I includes leucine, isoleucine, valine and methionine.

Group II includes glycine, alanine, valine and a modified amino acid having an ethyl side chain. Preferably, Group II includes glycine and alanine.

Group III includes phenylalanine, phenylglycine, tyrosine, tryptophan, cyclohexylmethyl glycine, and modified amino residues having substituted benzyl or phenyl side chains. Preferred substituents include one or more of the following: halogen, methyl, ethyl, nitro, —NH₂, methoxy, ethoxy and —CN. Preferably, Group III includes phenylalanine, tyrosine and tryptophan.

Group IV includes glutamic acid, aspartic acid, a substituted or unsubstituted aliphatic, aromatic or benzylic ester of glutamic or aspartic acid (e.g., methyl, ethyl, n-propyl iso-propyl, cyclohexyl, benzyl or substituted benzyl), glutamine, asparagine, O—NH— alkylated glutamine or asparagines (e.g., methyl, ethyl, n-propyl and iso-propyl) and modified amino acids having the side chain —(CH₂)₃—OOH, an ester thereof (substituted or unsubstituted aliphatic, aromatic or benzylic ester), an amide thereof and a substituted or unsubstituted N-alkylated amide thereof. Preferably, Group IV includes glutamic acid, aspartic acid, methyl aspartate, ethyl aspartate, benzyl aspartate and methyl glutamate, ethyl glutamate and benzyl glutamate, glutamine and asparagine.

Group V includes histidine, lysine, omithine, arginine, N-nitroarginine, β-cycloarginine, γ-hydroxyarginine, N-amidinocitruline and 2-amino-4-guanidinobutanoic acid, homologs of lysine, homologs of arginine and homologs of omithine. Preferably, Group V includes histidine, lysine, arginine and ornithine. A homolog of an amino acid includes from 1 to about 3 additional or subtracted methylene units in the side chain.

Group VI includes serine, threonine, cysteine and modified amino acids having C1-C5 straight or branched alkyl side chains substituted with —OH or —SH, for example, CH₂CH₂OH, CH₂CH₂CH₂OH or —CH₂CH₂OHCH₃. Preferably, Group VI includes serine, cysteine or threonine.

In another aspect, suitable substitutions for amino acid residues include “severe” substitutions. A “severe substitution” is a substitution in which the substituting amino acid (naturally occurring or modified) has significantly different size and/or electronic properties compared with the amino acid being substituted. Thus, the side chain of the substituting amino acid can be significantly larger (or smaller) than the side chain of the amino acid being substituted and/or can have functional groups with significantly different electronic properties than the amino acid being substituted. Examples of severe substitutions of this type include the substitution of phenylalanine or cyclohexylmethyl glycine for alanine, isoleucine for glycine, a D amino acid for the corresponding L amino acid, or —NH—CH[(—CH₂)₅—COOH]—CO— for aspartic acid. Alternatively, a functional group may be added to the side chain, deleted from the side chain or exchanged with another functional group. Examples of severe substitutions of this type include adding of valine, leucine or isoleucine, exchanging the carboxylic acid in the side chain of aspartic acid or glutamic acid with an amine, or deleting the amine group in the side chain of lysine or omithine. In yet another alternative, the side chain of the substituting amino acid can have significantly different steric and electronic properties that the functional group of the amino acid being substituted. Examples of such modifications include tryptophan for glycine, lysine for aspartic acid and —(CH₂)₄COOH for the side chain of serine. These examples are not meant to be limiting.

In another embodiment, for example in the synthesis of a peptide 26 amino acids in length, the individual amino acids may be substituted according in the following manner:

AA₁ is serine, glycine, alanine, cysteine or threonine;

AA₂ is alanine, threonine, glycine, cysteine or serine;

AA₃ is valine, arginine, leucine, isoleucine, methionine, omithine, lysine, N-nitroarginine, β-cycloarginine, γ-hydroxyarginine, N-amidinocitruline or 2-amino-4-guanidinobutanoic acid;

AA₄ is proline, leucine, valine, isoleucine or methionine;

AA₅ is tryptophan, alanine, phenylalanine, tyrosine or glycine;

AA₆ is serine, glycine, alanine, cysteine or threonine;

AA₇ is proline, leucine, valine, isoleucine or methionine;

AA₈ is alanine, threonine, glycine, cysteine or serine;

AA₉ is alanine, threonine, glycine, cysteine or serine;

AA₁₀ is leucine, isoleucine, methionine or valine;

AA₁₁ is serine, glycine, alanine, cysteine or threonine;

AA₁₂ is leucine, isoleucine, methionine or valine;

AA₁₃ is leucine, isoleucine, methionine or valine;

AA₁₄ is glutamine, glutamic acid, aspartic acid, asparagine, or a substituted or unsubstituted aliphatic or aryl ester of glutamic acid or aspartic acid;

AA₁₅ is arginine, N-nitroarginine, β-cycloarginine, γ-hydroxy-arginine, N-amidinocitruline or 2-amino-4-guanidino-butanoic acid

AA₁₆ is proline, leucine, valine, isoleucine or methionine;

AA₁₇ is serine, glycine, alanine, cysteine or threonine;

AA₁₈ is glutamic acid, aspartic acid, asparagine, glutamine or a substituted or unsubstituted aliphatic or aryl ester of glutamic acid or aspartic acid;

AA₁₉ is aspartic acid, asparagine, glutamic acid, glutamine, leucine, valine, isoleucine, methionine or a substituted or unsubstituted aliphatic or aryl ester of glutamic acid or aspartic acid;

AA₂₀ is valine, arginine, leucine, isoleucine, methionine, ornithine, lysine, N-nitroarginine, β-cycloarginine, γ-hydroxyarginine, N-amidinocitruline or 2-amino-4-guanidinobutanoic acid;

AA₂₁ is alanine, threonine, glycine, cysteine or serine;

AA₂₂ is alanine, threonine, glycine, cysteine or serine;

AA₂₃ is histidine, serine, threonine, cysteirie, lysine or ornithine;

AA₂₄ is threonine, aspartic acid, serine, glutamic acid or a substituted or unsubstituted aliphatic or aryl ester of glutamic acid or aspartic acid;

AA₂₅ is asparagine, aspartic acid, glutamic acid, glutamine, leucine, valine, isoleucine, methionine or a substituted or unsubstituted aliphatic or aryl ester of glutamic acid or aspartic acid; and

AA₂₆ is cysteine, hisfidine, serine, threonine, lysine or ornithine.

It is to be understood that these amino acid substitutions may be made for longer or shorter peptides than the 26 mer in the preceding example above, and for proteins.

In one embodiment of the present invention, codons for the first several N-terminal amino acids of the transposase are modified such that the third base of each codon is changed to an A or a T without changing the corresponding amino acid. It is preferable that between approximately 1 and 20, more preferably 3 and 15, and most preferably between 4 and 12 of the first N-terminal codons of the gene of interest are modified such that the third base of each codon is changed to an A or a T without changing the corresponding amino acid. In one embodiment, the first ten N-terminal codons of the gene of interest are modified in this manner.

When several desired proteins, protein fragments or peptides are encoded in the gene of interest to be incorporated into the genome, as with the multivalent multimeric proteins, one of skill in the art will appreciate that the proteins, protein fragments or peptides may be separated by a spacer molecule such as, for example, a peptide, consisting of one or more amino acids. Generally, the spacer will have no specific biological activity other than to join the desired proteins, protein fragments or peptides together, or to preserve some minimum distance or other spatial relationship between them. However, the constituent amino acids of the spacer may be selected to influence some property of the molecule such as the folding, net charge, or hydrophobicity. The spacer may also be contained within a nucleotide sequence with a purification handle or be flanked by proteolytic cleavage sites.

Such polypeptide spacers may have from about 1 to about 100 amino acids, preferably 3 to 20 amino acids, and more preferably 4-15 amino acids. The spacers in a polypeptide are independently chosen, but are preferably all the same. The spacers should allow for flexibility of movement in space and are therefore typically rich in small amino acids, for example, glycine, serine, proline or alanine. Preferably, peptide spacers contain at least 60%, more preferably at least 80% glycine or alanine. In addition, peptide spacers generally have little or no biological and antigenic activity. Preferred spacers are (Gly-Pro-Gly-Gly)ₓ (SEQ ID NO:81) and (Gly₄-Ser)_{y}, wherein x is an integer from about 3 to about 9 and y is an integer from about 1 to about 8. Specific examples of suitable spacers include

| | |
|---|---|
| (Gly-Pro-Gly-Gly)₃ | |
| Gly Pro Gly Gly Gly Pro Gly Gly Gly | SEQ ID NO: 82 |
| Pro Gly Gly (Gly₄-Ser)₃ | |
| Gly Gly Gly Gly Ser Gly Gly Gly Gly | SEQ ID NO: 83 |
| Ser Gly Gly Gly Gly Ser or | |
| (Gly₄-Ser)₄ | |
| Gly Gly Gly Gly Ser Gly Gly Gly Gly | SEQ ID NO: 84 |
| Ser Gly Gly Gly Gly Ser Gly Gly Gly | |
| Gly Ser. | |

One example of a multivalent multimeric protein containin a spacer is leutinizing hormone (LH), normally made as separate alpha and beta chains, made as a single polypeptide as described in Galet et. al., Mol. Cell Endocrinology, 2001, 174 (1-2):31-40. Production of a multimeric protein may thus be simplified using a spacer sequence that may or may not contain cleavage sites. In the case of an immunoglobulin, for example, a heavy and light chain may be synthesized as a single polypeptide using a spacer sequence with protease sites native to the transgenic animal so as to make, upon processing, a heavy and light chain combination in close association, facilitating the addition of a similar heavy and light chain to produce the native immunoglobulin. In this model, the removal of the spacer sequence may or may not be required. Other multimeric proteins may be made in bioengineered organisms in a similar fashion.

Nucleotide sequences encoding for the production of residues which may be useful in purification of the expressed recombinant protein may also be built into the vector. Such sequences are known in the art and include the glutathione binding domain from glutathione S-transferase, polylysine, hexa-histidine or other cationic amino acids, thioredoxin, hemagglutinin antigen and maltose binding protein.

Additionally, nucleotide sequences may be inserted into the gene of interest to be incorporated so that the protein or peptide can also include from one to about six amino acids that create signals for proteolytic cleavage. In this manner, if a gene is designed to make one or more peptides or proteins of interest in the transgenic animal, specific nucleotide sequences encoding for amino acids recognized by enzymes may be incorporated into the gene to facilitate cleavage of the large protein or peptide sequence into desired peptides or proteins or both. For example, nucleotides encoding a proteolytic cleavage site can be introduced into the gene of interest so that a signal sequence can be cleaved from a protein or peptide encoded by the gene of interest. Nucleotide sequences encoding other amino acid sequences which display pH sensitivity, chemical sensitivity or photolability may also be added to the vector to facilitate separation of the signal sequence from the peptide or protein of interest.

Proteolytic cleavage sites include cleavage sites recognized by exopeptidases such as carboxypeptidase A, carboxypeptidase B, aminopeptidase I, and dipeptidylaminopeptidase; endopeptidases such as trypsin, V8-protease, enterokinase, factor Xa, collagenase, endoproteinase, subtilisin, and thombin; and proteases such as Protease 3C IgA protease (Igase) Rhinovirus 3C(preScission)protease. Chemical cleavage sites are also included in the defintion of cleavage site as used herein. Chemical cleavage sites include, but are not limited to, site cleaved by cyanogen bromide, hydroxylamine, formic acid, and acetic acid. Self-splicing cleavage sites such as inteins are also included in the present invention.

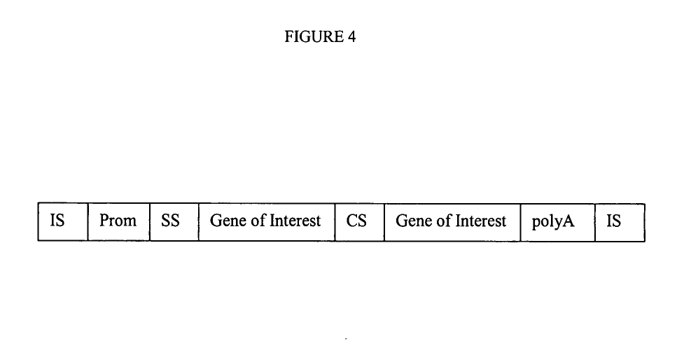

In some embodiments, one or more cleavage sites are incorporated into a polynucleotide cassette containing multiple genes of interest. FIG. 4 depicts one example of a polynucleotide cassette containing two genes of interest containing a cleavage site between them. The genes of interest may encode different proteins or peptides, the same protein or peptide, or modified versions of the same protein or peptide. While FIG. 4 shows a polynucleotide cassette containing two genes of interest, the present invention encompasses a polynucleotide cassette containing any number of genes of interest. The cleavage site located between the genes of interest can encode any amino acid sequence that is cleaved by any means. As mentioned above, the cleavage site can encode an amino acid sequence cleaved by a protease, a chemical reaction, can be a photolabile site, or can be a pro polynucleotide

The present invention includes a polynucleotide cassette that encodes a repetitive polypeptide chain in which two or more peptides, polypeptides or proteins, designated as P in the structural formulae presented below, are each separated by a peptide spacer or cleavage site designated as B. A polypeptide multivalent ligand, also called a multivalent protein, is a form of a multimeric protein encoded by the polynucleotide cassettes of the present invention, and is represented by structural formulae (I, II and III). Each peptide or protein is connected to another peptide or protein through a peptide bond, to a linker group, to a spacer, or to a cleavage site. Each peptide, polypeptide or protein may be the same or different and each linker, spacer, cleavage site or covalent bond is independently chosen.

A “polypeptide multivalent protein” is a multiple repeat polypeptide chain in which two or more peptides P are each separated by a peptide linker group, a spacer or a cleavage site. A polypeptide multivalent ligand is represented by structural formulae II and III.

B-(L-P)n I

wherein B is a peptide spacer or cleavage site, n is an integer from 2 to about 20, each L is a covalent bond, a linking group or cleavage site which may be present or absent, and each P is a peptide having from about 4 to about 200 amino acid residues.

P—(B—P)m-B—P II

wherein m is an integer from 0 to about 20.

Pa—(B)n-Pa III

wherein n is an integer from 1 to 20, preferably 2 to 10, more preferably 3 to 7, further wherein a is 1.

Other examples of multivalent proteins include the following:

P_{y}-Lₓ-Bₙ-Lₓ-Py IV

P_{y}—B—P_{y}

In the preceding structural formulae IV and V of polypeptide multivalent ligands encoded by a polynucleotide cassette of the present invention, each P is a peptide having from about 4 to about 200 amino acid residues, y is 1, x is an integer from 1 to 3, and n is an integer from 1 to 20, preferably 2 to 10, more preferably 3 to 7. Each B is a peptide spacer or cleavage site comprised of at least 2 amino acids or a cleavage site. Each peptide P and each B are independently chosen and may be the same or different.

Suitable linkers (L) are groups that can connect peptides and proteins to each other. In one example, the linker is an oligopeptide of from about 1 to about 10 amino acids consisting of amino acids with inert side chains. Suitable oligopeptides include polyglycine, polyserine, polyproline, polyalanine and oligopeptides consisting of alanyl and/or serinyl and/or prolinyl and/or glycyl amino acid residues. m in structural formula II is an integer from 0 to about 20.

The peptides, polypeptides and proteins in a multivalent protein can be connected to each other by covalent bonds, linker groups, spacers, cleavage groups or a combination thereof. The linking groups can be the same or different.

A polypeptide spacer shown in structural formula (II) is a peptide having from about 5 to about 40 amino acid residues. The spacers in a polypeptide multivalent ligand are independently chosen, and may be the same or different. The spacers should allow for flexibility of movement in space for the flanking peptides polypeptides and proteins P, and are therefore typically rich in small amino acids, for example, glycine, serine, proline or alanine. Preferably, peptide spacers contain at least 60%, more preferably at least 80% glycine or alanine. In addition, peptide spacers generally have little or no biological and antigenic activity. Preferred spacers are (Gly-Pro-Gly-Gly)ₓ (SEQ ID NO:81) and (Gly₄-Ser)_{y}, wherein x is an integer from about 3 to about 9 and y is an integer from about 1 to about 8. Specific examples of suitable spacers include (Gly₄-Ser)₃ (SEQ ID NO:82). Spacers can also include from one to about four amino acids that create a signal for proteolytic cleavage.

In another embodiment of the present invention, a TAG sequence is linked to a gene of interest. The TAG sequence serves three purposes: 1) it allows free rotation of the peptide or protein to be isolated so there is no interference from the native protein or signal sequence, i.e. vitellogenin, 2) it provides a “purification handle” to isolate the protein using affinity purification, and 3) it includes a cleavage site to remove the desired protein from the signal and purification sequences. Accordingly, as used herein, a TAG sequence includes a spacer sequence, a purification handle and a cleavage site. The spacer sequences in the TAG proteins contain one or more repeats shown in SEQ ID NO:85. A preferred spacer sequence comprises the sequence provided in SEQ ID NO:86. One example of a purification handle is the gp41 hairpin loop from HIV I. Exemplary gp41 polynucleotide and polypeptide sequences are provided in SEQ ID NO:87 and SEQ ID NO:88, respectively. However, it should be understood that any antigenic region, or otherwise associative regions such as avidin/biotin, may be used as a purification handle, including any antigenic region of gp41. Preferred purification handles are those that elicit highly specific antibodies. Additionally, the cleavage site can be any protein cleavage site known to one of ordinary skill in the art and includes an enterokinase cleavage site comprising the Asp Asp Asp Asp Lys sequence (SEQ ID NO:89) and a furin cleavage site. In one embodiment of the present invention, the TAG sequence comprises a polynucleotide sequence of SEQ ID NO:90.

Methods of Administering Polynucleotide Cassettes

In addition to the polynucleotide cassettes described above, the present invention also includes methods of administering the polynucleotide cassettes to an animal, methods of producing a transgenic animal wherein a gene of interest is incorporated into the germline of the animal and methods of producing a transgenic animal wherein a gene of interest is incorporated into cells other than the germline cells of the animal. The polynucleotide cassettes may reside in any vector or delivery solution when administered or may be naked DNA. In one embodiment, a transposon-based vector containing the polynucleotide cassette between two insertion sequences recognized by a transposase is administered to an animal. The polynucleotide cassettes of the present invention may be administered to an animal via any method known to those of skill in the art, including, but not limited to, intraembryonic, intratesticular, intraoviduct, intraovarian, into the duct system of the mammary gland, intraperitoneal, intraarterial, intravenous, topical, oral, nasal, and pronuclear injection methods of administration, or any combination thereof. The polynucleotide cassettes may also be administered within the lumen of an organ, into an organ, into a body cavity, into the cerebrospinal fluid, through the urinary system, through the genitourinary system, through the reproductive system, or through any route to reach the desired cells.

The polynucleotide cassettes may be delivered through the vascular system to be distributed to the cells supplied by that vessel. For example, the compositions may be placed in the artery supplying the ovary or supplying the fallopian tube to transfect cells in those tissues. In this manner, follicles could be transfected to create a germline transgenic animal. Alternatively, supplying the compositions through the artery leading to the oviduct would preferably transfect the tubular gland and epithelial cells. Such transfected cells could manufacture a desired protein or peptide for deposition in the egg white. Administration of the compositions through the portal vein would target uptake and transformation of hepatic cells. Administration through the urethra and into the bladder would target the transitional epithelium of the bladder. Administration through the vagina and cervix would target the lining of the uterus and the epithelial cells of the fallopian tube. Administration through the internal mammary artery or through the duct system of the mammary gland would transfect secretory cells of the lactating mammary gland to perform a desired function, such as to synthesize and secrete a desired protein or peptide into the milk.

The polynucleotide cassettes may be administered in a single administration, multiple administrations, continuously, or intermittently. The polynucleotide cassettes may be administered by injection, via a catheter, an osmotic mini-pump or any other method. In some embodiments, a polynucleotide cassette is administered to an animal in multiple administrations, each administration containing the polynucleotide cassette and a different transfecting reagent.

In a preferred embodiment, the animal is an egg-laying animal, and more preferably, an avian. In one embodiment, between approximately 1 and 150 μg, 1 and 100 μg, 1 and 50 μg, preferably between 1 and 20 μg, and more preferably between 5 and 10 μg of a transposon-based vector containing the polynucleotide cassette is administered to the oviduct of a bird. In a chicken, it is preferred that between approximately 1 and 100 μg, or 5 and 50 μg are administered. In a quail, it is preferred that between approximately S and 10 μg are administered. Optimal ranges depending upon the type of bird and the bird's stage of sexual maturity. Intraoviduct administration of the transposon-based vectors of the present invention result in a PCR positive signal in the oviduct tissue, whereas intravascular administration results in a PCR positive signal in the liver. In other embodiments, the polynucleotide cassettes is administered to an artery that supplies the oviduct or the liver. These methods of administration may also be combined with any methods for facilitating transfection, including without limitation, electroporation, gene guns, injection of naked DNA, and use of dimethyl sulfoxide (DMSO).

The transposon-based vectors may be administered to the animal at any point during the lifetime of the animal, however, it is preferable that the vectors are administered prior to the animal reaching sexual maturity. The transposon-based vectors are preferably administered to a chicken oviduct between approximately 14 and 16 weeks of age and to a quail oviduct between approximately 5 and 10 weeks of age, more preferably 5 and 8 weeks of age, and most preferably between 5 and 6 weeks of age, when standard poultry rearing practices are used. The vectors may be administered at earlier ages when exogenous hormones are used to induce early sexual maturation in the bird. In some embodiments, the transposon-based vector is administered to an animal's oviduct following an increase in proliferation of the oviduct epithelial cells and/or the tubular gland cells. Such an increase in proliferation normally follows an influx of reproductive hormones in the area of the oviduct. When the animal is an avian, the transposon-based vector is administered to the avian's oviduct following an increase in proliferation of the oviduct epithelial cells and before the avian begins to produce egg white constituents.

The present invention also includes a method of intraembryonic administration of a transposon-based vector containing a polynucleotide cassette to an avian embryo comprising the following steps: 1) incubating an egg on its side at room temperature for two hours to allow the embryo contained therein to move to top dead center (TDC); 2) drilling a hole through the shell without penetrating the underlying shell membrane; 3) injecting the embryo with the transposon-based vector in solution; 4) sealing the hole in the egg; and 5) placing the egg in an incubator for hatching. Administration of the transposon-based vector can occur anytime between immediately after egg lay (when the embryo is at Stage X) and hatching. Preferably, the transposon-based vector is administered between 1 and 7 days after egg lay, more preferably between 1 and 2 days after egg lay. The transposon-based vectors may be introduced into the embryo in amounts ranging from about 5.0 μg to 10 μg, preferably 1.0 μg to 100 μg. Additionally, the transposon-based vector solution volume may be between approximately 1 μl to 75 μl in quail and between approximately 1 μl to 500 μl in chicken.

The present invention also includes a method of intratesticular administration of a transposon-based vector containing a polynucleotide cassette including injecting a bird with a composition comprising the transposon-based vector, an appropriate carrier and an appropriate transfection reagent. In one embodiment, the bird is injected before sexual maturity, preferably between approximately 4-14 weeks, more preferably between approximately 6-14 weeks and most preferably between 8-12 weeks old. In another embodiment, a mature bird is injected with a transposon-based vector an appropriate carrier and an appropriate transfection reagent. The mature bird may be any type of bird, but in one example the mature bird is a quail.

A bird is preferably injected prior to the development of the blood-testis barrier, which thereby facilitates entry of the transposon-based vector into the seminiferous tubules and transfection of the spermatogonia or other germline cells. At and between the ages of 4, 6, 8, 10, 12, and 14 weeks, it is believed that the testes of chickens are likely to be most receptive to transfection. In this age range, the blood/testis barrier has not yet formed, and there is a relatively high number of spermatogonia relative to the numbers of other cell types, e.g., spermatids, etc. See J. Kumaran et al., 1949. Poultry Sci., 29:511-520. See also E. Oakberg, 1956. Am. J. Anatomy, 99:507-515; and P. Kluin et al., 1984. Anat. Embryol., 169:73-78.

The transposon-based vectors may be introduced into a testis in an amount ranging from about 0.1 μg to 10 μg, preferably 1 μg to 10 μg, more preferably 3 μg to 10 μg. In a quail, about 5 μg is a preferred amount. In a chicken, about 5 μg to 10 μg per testis is preferred. These amounts of vector DNA may be injected in one dose or multiple doses and at one site or multiple sites in the testis. In a preferred embodiment, the vector DNA is administered at multiple sites in a single testis, both testes being injected in this manner. In one embodiment, injection is spread over three injection sites: one at each end of the testis, and one in the middle. Additionally, the transposon-based vector solution volume may be between approximately 1 μl to 75 μl in quail and between approximately 1 μl to 500 μl in chicken. In a preferred embodiment, the transposon-based vector solution volume may be between approximately 20 μl to 60 μl in quail and between approximately 50 μl to 250 μl in chicken. Both the amount of vector DNA and the total volume injected into each testis may be determined based upon the age and size of the bird.

According to the present invention, the polynucleotide cassette is administered in conjunction with an acceptable carrier and/or transfection reagent. Acceptable carriers include, but are not limited to, water, saline, Hanks Balanced Salt Solution (HBSS), Tris-EDTA (TE) and lyotropic liquid crystals. Transfection reagents commonly known to one of ordinary skill in the art that may be employed include, but are not limited to, the following: cationic lipid transfection reagents, cationic lipid mixtures, polyamine reagents, liposomes and combinations thereof; SUPERFECT®, Cytofectene, BioPORTER®, GenePORTER®, NeuroPORTER®, and perfectin from Gene Therapy Systems; lipofectamine, cellfectin, DMRIE-C oligofectamine, and PLUS reagent from InVitrogen; Xtreme gene, fugene, DOSPER and DOTAP from Roche; Lipotaxi and Genejammer from Strategene; and Escort from SIGMA. In one embodiment, the transfecfion reagent is SUPERFECT®. The ratio of DNA to transfection reagent may vary based upon the method of administration. In one embodiment, a transposon-based vector containing a polynucleotide cassette is administered intratesticularly and the ratio of DNA to transfection reagent can be from 1:1.5 to 1:15, preferably 1:2 to 1:10, all expressed as wt/vol. Transfection may also be accomplished using other means known to one of ordinary skill in the art, including without limitation electroporation, gene guns, injection of naked DNA, and use of dimethyl sulfoxide (DMSO).

Depending upon the cell or tissue type targeted for transfection, the form of the transposon-based vector may be important. Plasmids harvested from bacteria are generally closed circular supercoiled molecules, and this is the preferred state of a vector for gene delivery because of the ease of preparation. In some instances, transposase expression and insertion may be more efficient in a relaxed, closed circular configuration or in a linear configuration. In still other instances, a purified transposase protein may be co-injected with a transposon-based vector containing the gene of interest for more immediate insertion. This could be accomplished by using a transfection reagent complexed with both the purified transposase protein and the transposon-based vector.

Testing for and Breeding Animals Carrying the Transgene

Following administration of a polynucleotide cassette to an animal, DNA is extracted from the animal to confirm integration of the genes of interest. Advantages provided by the present invention include the high rates of integration, or incorporation, and transcription of the gene of interest when administered to a bird via an intraoviduct or intraovary route (including intraarterial administrations to arteries leading to the oviduct or ovary) and contained within a transposon-based vector.

Actual frequencies of integration can be estimated both by comparative strength of the PCR signal, and by histological evaluation of the tissues by quantitative PCR. Another method for estimating the rate of transgene insertion is the so-called primed in situ hybridization technique (PRINS). This method determines not only which cells carry a transgene of interest, but also into which chromosome the gene has inserted, and even what portion of the chromosome. Briefly, labeled primers are annealed to chromosome spreads (affixed to glass slides) through one round of PCR, and the slides are then developed through normal in situ hybridization procedures. This technique combines the best features of in situ PCR and fluorescence in situ hybridization (FISH) to provide distinct chromosome location and copy number of the gene in question. The 28s rRNA gene will be used as a positive control for spermatogonia to confirm that the technique is functioning properly. Using different fluorescent labels for the transgene and the 28s gene causes cells containing a transgene to fluoresce with two different colored tags.

Breeding experiments may also be conducted to determine if germline transmission of the transgene has occurred. In a general bird breeding experiment performed according to the present invention, each male bird is exposed to 2-3 different adult female birds for 3-4 days each. This procedure is continued with different females for a total period of 6-12 weeks. Eggs are collected daily for up to 14 days after the last exposure to the transgenic male, and each egg is incubated in a standard incubator. The resulting embryos are examined for transgene presence at day 3 or 4 using PCR.

Any male producing a transgenic embryo is bred to additional females. Eggs from these females are incubated, hatched, and the chicks tested for the exogenous DNA. Any embryos that die are necropsied and examined directly for the transgene or protein encoded by the transgene, either by fluorescence or PCR. The offspring that hatch and are found to be positive for the exogenous DNA are raised to maturity. These birds are bred to produce further generations of transgenic birds, to verify efficiency of the transgenic procedure and the stable incorporation of the transgene into the germ line. The resulting embryos are examined for transgene presence at day 3 or 4 using PCR.

It is to be understood that the above procedure can be modified to suit animals other than birds and that selective breeding techniques may be performed to amplify gene copy numbers and protein output.

Production of Desired Multimeric Proteins in Egg White

In one embodiment, a transposon-based vector containing a polynucleotide cassette of the present invention may be administered to a bird for production of desired proteins or peptides in the egg white. These trasnposon-based vectors preferably contain one or more of an ovalbumin promoter, an ovomucoid promoter, an ovalbumin signal sequence and an ovomucoid signal sequence. Oviduct-specific ovalbumin promoters are described in B. O'Malley et al., 1987. EMBO J., vol. 6, pp. 2305-12; A. Qiu et al., 1994. Proc. Nat. Acad. Sci. (USA), vol. 91, pp. 4451-4455; D. Monroe et al., 2000. Biochim. Biophys. Acta, 1517 (1):27-32; H. Park et al., 2000. Biochem., 39:8537-8545; and T. Muramatsu et al., 1996. Poult. Avian Biol. Rev., 6:107-123.

Production of Desired Multimeric Proteins in Egg Yolk

The present invention is particularly advantageous for production of recombinant peptides and proteins of low solubility in the egg yolk. Such proteins include, but are not limited to, membrane-associated or membrane-bound proteins, lipophilic compounds; attachment factors, receptors, and components of second messenger transduction machinery. Low solubility peptides and proteins are particularly challenging to produce using conventional recombinant protein production techniques (cell and tissue cultures) because they aggregate in water-based, hydrophilic environments. Such aggregation necessitates denaturation and re-folding of the recombinantly-produced proteins, which may deleteriously affect their structure and function. Moreover, even highly soluble recombinant peptides and proteins may precipitate and require denaturation and renaturation when produced in sufficiently high amounts in recombinant protein production systems. The present invention provides an advantageous resolution of the problem of protein and peptide solubility during production of large amounts of recombinant proteins.

In one embodiment of the present invention, deposition of a desired protein into the egg yolk is accomplished by attaching a sequence encoding a protein capable of binding to the yolk vitellogenin receptor to a gene of interest that encodes a desired protein. This polynucleotide cassette can be used for the receptor-mediated uptake of the desired protein by the oocytes. In a preferred embodiment, the sequence ensuring the binding to the vitellogenin receptor is a targeting sequence of a vitellogenin protein. The invention encompasses various vitellogenin proteins and their targeting sequences. In a preferred embodiment, a chicken vitellogenin protein targeting sequence is used, however, due to the high degree of conservation among vitellogenin protein sequences and known cross-species reactivity of vitellogenin targeting sequences with their egg-yolk receptors, other vitellogenin targeting sequences can be substituted. One example of a construct for use in the transposon-based vectors of the present invention and for deposition of an insulin protein in an egg yolk is a transposon-based vector containing a vitellogenin promoter, a vitellogenin targeting sequence, a TAG sequence, a pro-insulin sequence and a synthetic polyA sequence. The present invention includes, but is not limited to, vitellogenin targeting sequences residing in the N-terminal domain of vitellogenin, particularly in lipovitellin I. In one embodiment, the vitellogenin targeting sequence contains the polynucleotide sequence of SEQ ID NO:77.

In a preferred embodiment, the transposon-based vector contains a transposase gene operably-linked to a constitutive promoter and a gene of interest operably-linked to a liver-specific promoter and a vitellogenin targeting sequence.

Isolation and Purification of Desired Multimeric Proteins

For large-scale production of protein, an animal breeding stock that is homozygous for the transgene is preferred. Such homozygous individuals are obtained and identified through, for example, standard animal breeding procedures or PCR protocols.

Once expressed, peptides, polypeptides and proteins can be purified according to standard procedures known to one of ordinary skill in the art, including ammonium sulfate precipitation, affinity columns, column chromatography, gel electrophoresis, high performance liquid chromatography, immunoprecipitation and the like. Substantially pure compositions of about 50 to 99% homogeneity are preferred, and 80 to 95% or greater homogeneity are most preferred for use as therapeutic agents.

In one embodiment of the present invention, the animal in which the desired protein is produced is an egg-laying animal. In a preferred embodiment of the present invention, the animal is an avian and a desired peptide, polypeptide or protein is isolated from an egg white. Egg white containing the exogenous protein or peptide is separated from the yolk and other egg constituents on an industrial scale by any of a variety of methods known in the egg industry. See, e.g., W. Stadelman et al. (Eds.), Egg Science & Technology, Haworth Press, Binghamton, N.Y. (1995). Isolation of the exogenous peptide or protein from the other egg white constituents is accomplished by any of a number of polypeptide isolation and purification methods well known to one of ordinary skill in the art. These techniques include, for example, chromatographic methods such as gel permeation, ion exchange, affinity separation, metal chelation, HPLC, and the like, either alone or in combination. Another means that may be used for isolation or purification, either in lieu of or in addition to chromatographic separation methods, includes electrophoresis. Successful isolation and purification is confirmed by standard analytic techniques, including HPLC, mass spectroscopy, and spectrophotometry. These separation methods are often facilitated if the first step in the separation is the removal of the endogenous ovalbumin fraction of egg white, as doing so will reduce the total protein content to be further purified by about 50%.

To facilitate or enable purification of a desired protein or peptide, the polynucleotide cassettes may include one or more additional epitopes or domains. Such epitopes or domains include DNA sequences encoding enzymatic, chemical or photolabile cleavage sites including, but not limited to, an enterokinase cleavage site; the glutathione binding domain from glutathione S-transferase; polylysine; hexa-histidine or other cationic amino acids, and sites cleaved by cyanogen bromide, hydroxylamine, formic acid, and acetic acid; thioredoxin; hemagglutinin antigen; maltose binding protein; a fragment of gp41 from HIV; and other purification epitopes or domains commonly known to one of skill in the art. Other proteolytic cleavage sites that may be included in the polynucleotide cassettes are cleavage sites recognized by exopeptidases such as carboxypeptidase A, carboxypeptidase B, aminopeptidase I, and dipeptidylaminopeptidase; endopeptidases such as trypsin, V8-protease, enterokinase, factor Xa, collagenase, endoproteinase, subtilisin, and thombin; and proteases such as Protease 3C IgA protease (Igase) Rhinovirus 3C(preScission)protease. Self-splicing cleavage sites such as inteins may also be included in the polynucleotide cassettes of the present invention.

In one representative embodiment, purification of desired proteins from egg white utilizes the antigenicity of the ovalbumin carrier protein and particular attributes of a TAG linker sequence that spans ovalbumin and the desired protein. The TAG sequence is particularly useful in this process because it contains 1) a highly antigenic epitope, a fragment of gp41 from HIV, allowing for stringent affinity purification, and, 2) a recognition site for the protease enterokinase immediately juxtaposed to the desired protein. In a preferred embodiment, the TAG sequence comprises approximately 50 amino acids. A representative TAG sequence is provided below.

| | |
|---|---|
| Pro Ala Asp Asp Ala Pro Ala Asp | (SEQ ID NO: 90) |
| Asp Ala Pro Ala Asp Asp Ala Pro | |
| Ala Asp Asp Ala Pro Ala Asp Asp | |
| Ala Pro Ala Asp Asp Ala Thr Thr | |
| Cys Ile Leu Lys Gly Ser Cys Gly | |
| Trp Ile Gly Leu Leu Asp Asp Asp | |
| Asp Lys | |

The underlined sequences were taken from the hairpin loop domain of HIV gp-41 (SEQ ID NO:87). Sequences in italics represent the cleavage site for enterokinase (SEQ ID NO:89). The spacer sequence upstream of the loop domain was made from repeats of (Pro Ala Asp Asp Ala) (SEQ ID NO:85) to provide free rotation and promote surface availability of the hairpin loop from the ovalbumin carrier protein.

Isolation and purification of a desired protein is performed as follows:

1. Enrichment of the egg white protein fraction containing ovalbumin and the transgenic ovalbumin-TAG-desired protein.

2. Size exclusion chromatography to isolate only those proteins within a narrow range of molecular weights (a further enrichment of step 1).

3. Ovalbumin affinity chromatography. Highly specific antibodies to ovalbumin will eliminate virtually all extraneous egg white proteins except ovalbumin and the transgenic ovalbumin-TAG-desired protein.

4. gp41 affinity chromatography using anti-gp41 antibodies. Stringent application of this step will result in virtually pure transgenic ovalbumin-TAG-desired protein.

5. Cleavage of the transgene product can be accomplished in at least one of two ways:

a. The transgenic ovalbumin-TAG-desired protein is left attached to the gp41 affinity resin (beads) from step 4 and the protease enterokinase is added. This liberates the transgene target protein from the gp41 affinity resin while the ovalbumin-TAG sequence is retained. Separation by centrifugation (in a batch process) or flow through (in a column purification), leaves the desired protein together with enterokinase in solution. Enterokinase is recovered and reused.

b. Alternatively, enterokinase is immobilized on resin (beads) by the addition of poly-lysine moieties to a non-catalytic area of the protease. The transgenic ovalbumin-TAG-desired protein eluted from the affinity column of step 4 is then applied to the protease resin. Protease action cleaves the ovalbumin-TAG sequence from the desired protein and leaves both entities in solution. The immobilized enterokinase resin is recharged and reused.

c. The choice of these alternatives is made depending upon the size and chemical composition of the transgene target protein.

6. A final separation of either of these two (5a or 5b) protein mixtures is made using size exclusion, or enterokinase affinity chromatography. This step allows for desalting, buffer exchange and/or polishing, as needed.

Cleavage of the transgene product (ovalbumin-TAG-desired protein) by enterokinase, then, results in two products: ovalbumin-TAG and the desired protein. More specific methods for isolation using the TAG label is provided in the Examples. Some desired proteins may require additions or modifications of the above-described approach as known to one of ordinary skill in the art. The method is scaleable from the laboratory bench to pilot and production facility largely because the techniques applied are well documented in each of these settings.

It is believed that a typical chicken egg produced by a transgenic animal of the present invention will contain at least 0.001 mg, from about 0.001 to 1.0 mg, or from about 0.001 to 100.0 mg of exogenous protein, peptide or polypeptide, in addition to the normal constituents of egg white (or possibly replacing a small fraction of the latter).

One of skill in the art will recognize that after biological expression or purification, the desired proteins, fragments thereof and peptides may possess a conformation substantially different than the native conformations of the proteins, fragments thereof and peptides. In this case, it is often necessary to denature and reduce protein and then to cause the protein to re-fold into the preferred conformation. Methods of reducing and denaturing proteins and inducing re-folding are well known to those of skill in the art.

Production of Multimeric Proteins in Milk

In addition to methods of producing eggs containing transgenic proteins or peptides, the present invention encompasses methods for the production of milk containing transgenic proteins or peptides. These methods include the administration of a transposon-based vector described above to a mammal. In one embodiment, the transposon-based vector contains a transposase operably-linked to a constitutive promoter and a gene of interest operably-linked to mammary specific promoter. Genes of interest can include, but are not limited to antiviral and antibacterial proteins and immunoglobulins.

The following examples will serve to further illustrate the present invention without, at the same time, however, constituting any limitation thereof. On the contrary, it is to be clearly understood that resort may be had to various embodiments, modifications and equivalents thereof which, after reading the description herein, may suggest themselves to those skilled in the art without departing from the spirit of the invention.

### EXAMPLE 1

Preparation of Transposon-Based Vector pTnMod

A vector was designed for inserting a desired coding sequence into the genome of eukaryotic cells, given below as SEQ ID NO:57. The vector of SEQ ID NO:57, termed pTnMod, was constructed and its sequence verified.

This vector employed a cytomegalovirus (CMV) promoter. A modified Kozak sequence (ACCATG) (SEQ ID NO:5) was added to the promoter. The nucleotide in the wobble position in nucleotide triplet codons encoding the first 10 amino acids of transposase was changed to an adenine (A) or thymine (T), which did not alter the amino acid encoded by this codon. Two stop codons were added and a synthetic polyA was used to provide a strong termination sequence. This vector uses a promoter designed to be active soon after entering the cell (without any induction) to increase the likelihood of stable integration. The additional stop codons and synthetic polyA insures proper termination without read through to potential genes downstream.

The first step in constructing this vector was to modify the transposase to have the desired changes. Modifications to the transposase were accomplished with the primers High Efficiency forward primer (Hef) Altered transposase (ATS)-Hef 5′ ATCTCGAGACCATGTGTGAACTTGATATTTTACATGATTCTCTTTACC 3′ (SEQ ID NO:91) and Altered transposase-High efficiency reverse primer (Her) 5′ GATTGATCATTATCATAATTTCCCCAAAGCGTAACC 3′ (SEQ ID NO:92, a reverse complement primer). In the 5′ forward primer ATS-Hef, the sequence CTCGAG (SEQ ID NO:93) is the recognition site for the restriction enzyme Xho I, which permits directional cloning of the amplified gene. The sequence ACCATG (SEQ ID NO:5) contains the Kozak sequence and start codon for the transposase and the underlined bases represent changes in the wobble position to an A or T of codons for the first 10 amino acids (without changing the amino acid coded by the codon). Primer ATS-Her (SEQ ID NO:92) contains an additional stop codon TAA in addition to native stop codon TGA and adds a Bcl I restriction site, TGATCA (SEQ ID NO:94), to allow directional cloning. These primers were used in a PCR reaction with pTnLac (p defines plasmid, tn defines transposon, and lac defines the beta fragment of the lactose gene, which contains a multiple cloning site) as the template for the transposase and a FailSafe™ PCR System (which includes enzyme, buffers, dNTP's, MgCl₂ and PCR Enhancer; Epicentre Technologies, Madison, Wis.). Amplified PCR product was electrophoresed on a 1% agarose gel, stained with ethidium bromide, and visualized on an ultraviolet transilluminator. A band corresponding to the expected size was excised from the gel and purified from the agarose using a Zymo Clean Gel Recovery Kit (Zymo Research, Orange, Calif.). Purified DNA was digested with restriction enzymes Xho I (5′) and Bcl 1 (3′) (New England Biolabs, Beverly, Mass.) according to the manufacturer's protocol. Digested DNA was purified from restriction enzymes using a Zymo DNA Clean and Concentrator kit (Zymo Research).

Plasmid gWhiz (Gene Therapy Systems, San Diego, Calif.) was digested with restriction enzymes Sal I and BamH I (New England Biolabs), which are compatible with Xho I and Bcl I, but destroy the restriction sites. Digested gwhiz was separated on an agarose gel, the desired band excised and purified as described above. Cutting the vector in this manner facilitated directional cloning of the modified transposase (mATS) between the CMV promoter and synthetic polyA.

To insert the mATS between the CMV promoter and synthetic polyA in gwhiz, a Stratagene T4 Ligase Kit (Stratagene, Inc. La Jolla, Calif.) was used and the ligation set up according to the manufacturer's protocol. Ligated product was transformed into E. coli Top 10 competent cells (Invitrogen Life Technologies, Carlsbad, Calif.) using chemical transformation according to Invitrogen's protocol. Transformed bacteria were incubated in 1 ml of SOC (GIBCO BRL, CAT# 15544-042) medium for 1 hour at 37° C. before being spread to LB (Luria-Bertani media (broth or agar)) plates supplemented with 100 μg/ml ampicillin (LB/amp plates). These plates were incubated overnight at 37° C. and resulting colonies picked to LB/amp broth for overnight growth at 37° C. Plasmid DNA was isolated using a modified alkaline lysis protocol (Sambrook et al., 1989), electrophoresed on a 1% agarose gel, and visualized on a U.V. transilluminator after ethidium bromide staining. Colonies producing a plasmid of the expected size (approximately 6.4 kbp) were cultured in at least 250 ml of LB/amp broth and plasmid DNA harvested using a Qiagen Maxi-Prep Kit (column purification) according to the manufacturer's protocol (Qiagen, Inc., Chatsworth, Calif.). Column purified DNA was used as template for sequencing to verify the changes made in the transposase were the desired changes and no further changes or mutations occurred due to PCR amplification. For sequencing, Perkin-Elmer's Big Dye Sequencing Kit was used. All samples were sent to the Gene Probes and Expression Laboratory (LSU School of Veterinary Medicine) for sequencing on a Perkin-Elmer Model 377 Automated Sequencer.

Once a clone was identified that contained the desired mATS in the correct orientation, primers CMVf-NgoM IV (5′ TTGCCGGCATCAGATTGGCTAT (SEQ ID NO:95); underlined bases denote NgoM IV recognition site) and Syn-polyA-BstE II (5′ AGAGGTCACCGGGTCAATTCTTCAGCACCTGGTA (SEQ ID NO:96); underlined bases denote BstE II recognition site) were used to PCR amplify the entire CMV promoter, mATS, and synthetic polyA for cloning upstream of the transposon in pTnLac. The PCR was conducted with FailSafe™ as described above, purified using the Zymo Clean and Concentrator kit, the ends digested with NgoM IV and BstE II (New England Biolabs), purified with the Zymo kit again and cloned upstream of the transposon in pTnLac as described below.

Plasmid pTnLac was digested with NgoM IV and BstE II to remove the ptac promoter and transposase and the fragments separated on an agarose gel. The band corresponding to the vector and transposon was excised, purified from the agarose, and dephosphorylated with calf intestinal alkaline phosphatase (New England Biolabs) to prevent self-annealing. The enzyme was removed from the vector using a Zymo DNA Clean and Concentrator-5. The purified vector and CMVp/mATS/polyA were ligated together using a Stratagene T4 Ligase Kit and transformed into E. coli as described above.

Colonies resulting from this transformation were screened (mini-preps) as describe above and clones that were the correct size were verified by DNA sequence analysis as described above. The vector was given the name pTnMod (SEQ ID NO:57) and includes the following components:

Base pairs 1-130 are a remainder of F1(−) on from pBluescriptll sk(−) (Stratagene), corresponding to base pairs 1-130 of pBluescriptll sk(−).

Base pairs 131-132 are a residue from ligation of restriction enzyme sites used in constructing the vector.

Base pairs 133-1777 are the CMV promoter/enhancer taken from vector pGWiz (Gene Therapy Systems), corresponding to bp 229-1873 of pGWiz. The CMV promoter was modified by the addition of an ACC sequence upstream of ATG.

Base pairs 1778-1779 are a residue from ligation of restriction enzyme sites used in constructing the vector.

Base pairs 1780-2987 are the coding sequence for the transposase, modified from Tn10 (GenBank accession J01829) by optimizing codons for stability of the transposase mRNA and for the expression of protein. More specifically, in each of the codons for the first ten amino acids of the transposase, G or C was changed to A or T when such a substitution would not alter the amino acid that was encoded.

Base pairs 2988-2993 are two engineered stop codons.

Base pair 2994 is a residue from ligation of restriction enzyme sites used in constructing the vector.

Base pairs 2995-3410 are a synthetic polyA sequence taken from the pGWiz vector (Gene Therapy Systems), corresponding to bp 1922-2337 of 10 pGWiz.

Base pairs 3415-3718 are non-coding DNA that is residual from vector pNK2859.

Base pairs 3719-3761 are non-coding λ DNA that is residual from pNK2859.

Base pairs 3762-3831 are the 70 bp of the left insertion sequence recognized by the transposon Tn10.

Base pairs 3832-3837 are a residue from ligation of restriction enzyme sites used in constructing the vector.

Base pairs 3838-4527 are the multiple cloning site from pBluescriptll sk(20), corresponding to bp 924-235 of pBluescriptll sk(−). This multiple cloning site may be used to insert any coding sequence of interest into the vector.

Base pairs 4528-4532 are a residue from ligation of restriction enzyme sites used in constructing the vector.

Base pairs 4533-4602 are the 70 bp of the right insertion sequence recognized by the transposon Tn10.

Base pairs 4603-4644 are non-coding λ DNA that is residual from pNK2859.

Base pairs 4645-5488 are non-coding DNA that is residual from pNK2859.

Base pairs 5489-7689 are from the pBluescriptll sk(−) base vector—(Stratagene, Inc.), corresponding to bp 761-2961 of pBluescriptll sk(−).

Completing pTnMod is a pBlueScript backbone that contains a colE I origin of replication and an antibiotic resistance marker (ampicillin).

It should be noted that all non-coding DNA sequences described above can be replaced with any other non-coding DNA sequence(s). Missing nucleotide sequences in the above construct represent restriction site remnants.

All plasmid DNA was isolated by standard procedures. Briefly, Escherichia coli containing the plasmid was grown in 500 mL aliquots of LB broth (supplemented with an appropriate antibiotic) at 37° C. overnight with shaking. Plasmid DNA was recovered from the bacteria using a Qiagen Maxi-Prep kit (Qiagen, Inc., Chatsworth, Calif.) according to the manufacturer's protocol. Plasmid DNA was resuspended in 500 μL of PCR-grade water and stored at −20° C. until used.

### EXAMPLE 2

Transposon-Based Vector pTnMCS

Another transposon-based vector was designed for inserting a desired coding sequence into the genome of eukaryotic cells. This vector was termed pTnMCS and its constituents are provided below. The sequence of the pTnMCS vector is provided in SEQ ID NO:56. The pTnMCS vector contains an avian optimized polyA sequence operably-linked to the transposase gene. The avian optimized polyA sequence contains approximately 75 nucleotides that precede the A nucleotide string.

Bp 1-130 Remainder of F1 (−) ori of pBluescriptll sk(−) (Stratagene) bpl-130

Bp 133-1777 CMV promoter/enhancer taken from vector pGWIZ (Gene Therapy Systems) bp 229-1873

Bp 1783-2991 Transposase, from Tn10 (GenBank accession #J01829) bp 108-1316

Bp 2992-3344 Non coding DNA from vector pNK2859

Bp 3345-3387 Lambda DNA from pNK2859

Bp 3388-3457 70 bp of IS10 left from Tn10

Bp 3464-3670 Multiple cloning site from pBluescriptII sk(−), thru the XmaI site bp 924-718

Bp 3671-3715 Multiple cloning site from pBluescriptll sk(−), from the XmaI site thru the XhoI site. These base pairs are usually lost when cloning into pTnMCS bp 717-673

Bp 3716-4153 Multiple cloning site from pbluescriptII sk(−), from the XhoI site bp 672-235

Bp 4159-4228 70 bp of IS10 right from Tn10

Bp 4229-4270 Lambda DNA from pNK2859

Bp 4271-5114 Non-coding DNA from pNK2859

Bp 5115-7315 pbluescript sk (−) base vector (Stratagene, Inc.) bp 761-2961.

### EXAMPLE 3

Production of Antibody in Egg White

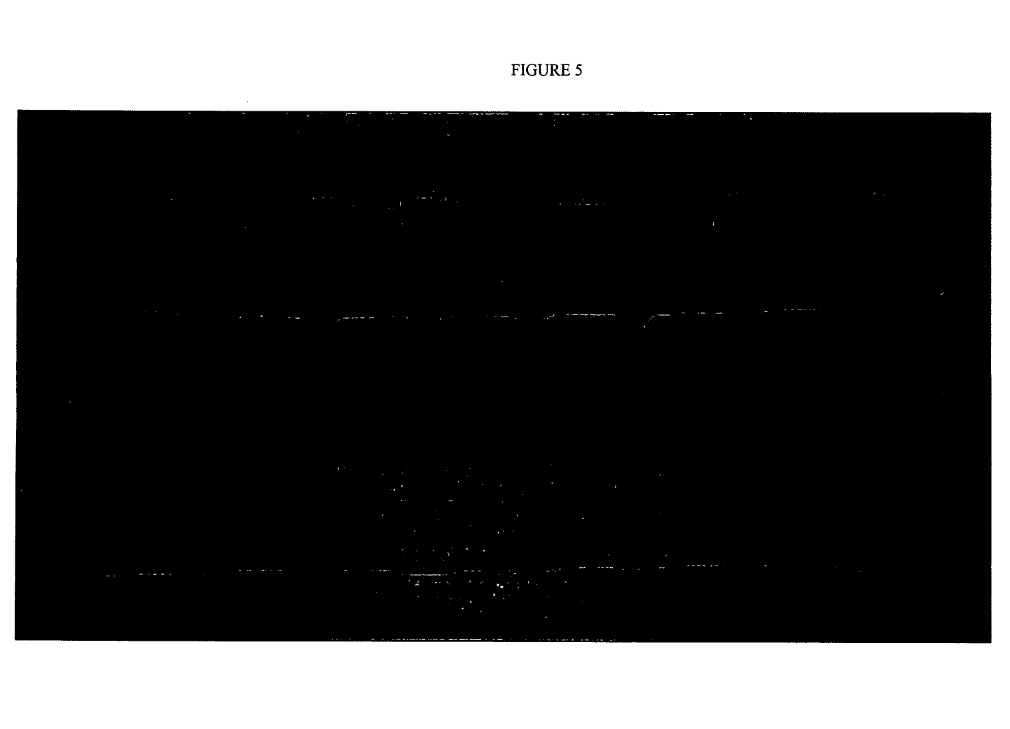

A transposon-based vector containing a CMV promoter/cecropin prepro/antibody heavy chain/cecropin pro/Antibody light chain/conalbumin poly A (SEQ ID NO:97) was injected into the oviduct of quail and chickens. A total of 20 birds were injected (10 chickens and 10 quail) and eggs were harvested from the birds once the eggs were laid. Partially purified egg white protein (EW) was then run on a gel under both reducing and non-reducing conditions. FIG. 5 is a picture of the gel. Lanes 1 & 18: molecular weight markers, Lanes 2 and 3: EW #1, non-reduced, reduced, respectively; Lanes 4 and 5: EW #2, non-reduced, reduced, respectively, Lanes 6 and 7: EW #3, non-reduced, reduced, respectively, Lanes 8 and 9: EW #4, non-reduced, reduced, respectively; Lanes 10 and 11: EW #5, non-reduced, reduced, respectively; Lanes 12 and 13: EW #6, non-reduced, reduced, respectively; Lanes 14 and 15: EW #7, non-reduced, reduced, respectively; and Lanes 16 and 17: EW #8 Control, non-reduced, reduced, respectively. Based upon the gel results, the possibility that the egg white in the treated chicken and quail contains antibody produced by the above-mentioned transposon-based vector cannot be excluded.

### EXAMPLE 4

Additional Transpson-Based Vectors for Administration to an Animal

The following example provides a description of various transposon-based vectors of the present invention and several constructs for insertion into the transposon-based vectors of the present invention. These examples are not meant to be limiting in any way. The constructs for insertion into a transposon-based vector are provided in a cloning vector pTnMCS or pTnMod, both described above.

pTnMOD (CMV-prepro-HCPro-Lys-CPA) (SEQ ID NO:97)

Bp 1-4090 from vector pTnMod, bp 1-4090

Bp 4096-5739 CMV promoter/enhancer taken from vector pGWIZ (Gene therapy systems), bp 230-1864

Bp 5746-5916 Capsite/Prepro taken from GenBank accession # X07404, bp 563-733

Bp 5923-7287 Heavy Chain gene construct taken from antibody RM2 provided by Mark Glassy (Shantha West, Inc)

Bp 7288-7302 Pro taken from GenBank accession # X07404, bp 719-733 (includes Lysine)

Bp 7309-7953 Light Chain gene construct taken from antibody RM2 provided by Mark Glassy (Shantha West, Inc)

Bp 7960-8372 Conalbumin polyA taken from GenBank accession # Y00407, bp 10651-11058

Bp 8374-11973 from cloning vector pTnMod, bp 4091-7690

pTnMCS(CHOVep-prepro-HCPro-CPA) (SEQ ID NO:98)

Bp 1-3715 from vectorpTnMCS, bp 1-3715

Bp 3721-4395 Chicken Ovalbumin enhancer taken from GenBank accession # S82527.1, bp 1-675

Bp 4402-5738 Chicken Ovalbumin promoter taken from GenBank accession # J00899-M24999, bp 1-1336

Bp 5745-5915 Capsite/Prepro taken from GenBank accession # X07404, bp 563-733

Bp 5922-7286 Heavy Chain gene construct taken from antibody RM2 provided by Mark Glassy (Shantha West, Inc)

Bp 7287-7298 Pro taken from GenBank accession # X07404, bp 719-730 (does not include Lysine)

Bp 7305-7949 Light Chain gene construct taken from antibody RM2 provided by Mark Glassy (Shantha West, Inc)

Bp 7956-8363 Conalbumin polyA taken from GenBank accession # Y00407, bp 10651-11058

Bp 8365-11964 from cloning vector pTnMCS, bp 3716-7315

pTnMCS(CHOvep-prepro-HCPro-Lys-CPA) (SEQ ID NO:99)

Bp 1-3715 from vector pTnMCS, bp 1-3715

Bp 3721-4395 Chicken Ovalbumin enhancer taken from GenBank accession # S82527.1, bp 1-675

Bp 4402-5738 Chicken Ovalbumin promoter taken from GenBank accession # J00899-M24999, bp 1-1336

Bp 5745-5915 Capsite/Prepro taken from GenBank accession # X07404, bp 563-733

Bp 5922-7286 Heavy Chain gene construct taken from antibody RM2 provided by Mark Glassy (Shantha West, Inc)

Bp 7287-7301 Pro taken from GenBank accession # X07404, bp 719-733 (includes Lysine)

Bp 7308-7952 Light Chain gene construct taken from antibody RM2 provided by Mark Glassy (Shantha West, Inc)

Bp 7959-8366 Conalbumin polyA taken from GenBank accession # Y00407, bp 10651-11058

Bp 8368-11967 from cloning vector pTnMCS, bp 3716-7315

pTnMCS (CMV-prepro-HCPro-CPA) (SEQ ID NO:100)

Bp 1-3715 from vector pTnMCS, bp 1-3715

Bp 3721-5364 CMV promoter/enhancer taken from vector pGWIZ (Gene therapy systems), bp 230-1864

Bp 5371-5541 Capsite/Prepro taken from GenBank accession # X07404, bp 563-733

Bp 5548-6912 Heavy Chain gene construct taken from antibody RM2 provided by Mark Glassy (Shantha West, Inc)

Bp 6913-6924 Pro taken from GenBank accession # X07404, bp 719-730 (does not Lysine)

Bp 6931-7575 Light Chain gene construct taken from antibody RM2 provided by Mark Glassy (Shantha West, Inc)

Bp 7582-7989 Conalbumin polyA taken from GenBank accession # Y00407, bp 10651-11058

Bp 7991-11590 from cloning vector pTnMCS, bp 3716-7315

pTnMCS (CMV-prepro-HCPro-Lys-CPA) (SEQ ID NO:101)

Bp 1-3715 from vector pTnMCS, bp 1-3715

Bp 3721-5364 CMV promoter/enhancer taken from vector pGWIZ (Gene therapy systems), bp 230-1864

Bp 5371-5541 Capsite/Prepro taken from GenBank accession # X07404, bp 563-733

Bp 5548-6912 Heavy Chain gene construct taken from antibody RM2 provided by Mark Glassy (Shantha West, Inc)

Bp 6913-6927 Pro taken from GenBank accession # X07404, bp 719-733 (includes Lysine)

Bp 6934-7578 Light Chain gene construct taken from antibody RM2 provided by Mark Glassy (Shantha West, Inc)

Bp 7585-7992 Conalbumin polyA taken from GenBank accession # Y00407, bp 10651-11058

Bp 7994-11593 from cloning vector pTnMCS, bp 3716-7315

pTnMod (CHOvep-prepro-HCPro-CPA) (SEQ ID NO:102)

Bp 1-4090 from vector pTnMod, bp 1-4090

Bp 4096-4770 Chicken Ovalbumin enhancer taken from GenBank accession # S82527.1, bp 1-675

Bp 4777-6113 Chicken Ovalbumin promoter taken from GenBank accession # J00899-M24999, bp 1-1336

Bp 6120-6290 Capsite/Prepro taken from GenBank accession # X07404, bp 563-733

Bp 6297-7661 Heavy Chain gene construct taken from antibody RM2 provided by Mark Glassy (Shantha West, Inc)

Bp 7662-7673 Pro taken from GenBank accession # X07404, bp 719-730 (does not include Lysine)

Bp 7680-8324 Light Chain gene construct taken from antibody RM2 provided by Mark Glassy (Shantha West, Inc)

Bp 8331-8738 Conalbumin polyA taken from GenBank accession # Y00407, bp 10651-11058

Bp 8740-12339 from cloning vector pTnMod, bp 3716-7315

pTnMod (CHOvep-prepro-HCPro-LYS-CPA) (SEQ ID NO:103)

Bp 1-4090 from vector pTnMod, bp 14090

Bp 4096-4770 Chicken Ovalbumin enhancer taken from GenBank accession # S82527.1, bp 1-675

Bp 4777-6113 Chicken Ovalbumin promoter taken from GenBank accession # J00899-M24999, bp 1-1336

Bp 6120-6290 Capsite/Prepro taken from GenBank accession # X07404, bp 563-733

Bp 6297-7661 Heavy Chain gene construct taken from antibody RM2 provided by Mark Glassy (Shantha West, Inc)

Bp 7662-7676 Pro taken from GenBank accession # X07404, bp 719-733 (includes Lysine)

Bp 7683-8327 Light Chain gene construct taken from antibody RM2 provided by Mark Glassy (Shantha West, Inc)

Bp 8334-8741 Conalbumin polyA taken from GenBank accession # Y00407, bp 10651-11058

Bp 8743-12342 from cloning vector pTnMod, bp 3716-7315

pTnMod (CMV-prepro-HCPro-CPA) (SEQ ID NO:104)

Bp 1-4090 from vector pTnMod, bp 1-4090

Bp 4096-5739 CMV promoter/enhancer taken from vector pGWIZ (Gene therapy systems), bp 230-1864

Bp 5746-5916 Capsite/Prepro taken from GenBank accession # X07404, bp 563-733

Bp 5923-7287 Heavy Chain gene construct taken from antibody RM2 provided by Mark Glassy (Shantha West, Inc)

Bp 7288-7299 Pro taken from GenBank accession # X07404, bp 719-730 (does not include Lysine)

Bp 7306-7950 Light Chain gene construct taken from antibody RM2 provided by Mark Glassy (Shantha West, Inc)

Bp 7557-7969 Conalbumin polyA taken from GenBank accession # Y00407, bp 10651-11058

Bp 7971-11970 from cloning vector pTnMod, bp 3716-7315

All patents, publications and abstracts cited above are incorporated herein by reference in their entirety. It should be understood that the foregoing relates only to preferred embodiments of the present invention and that numerous modifications or alterations may be made therein without departing from the spirit and the scope of the present invention as defined in the following claims.

We claim:

1. An isolated polynucleotide comprising two or more genes of interest and two or more pro nucleotide sequences, wherein each gene of interest is operably-linked to a pro nucleotide sequence and each of the two or more genes of interest may be the same or different.

2. The polynucleotide of claim 1, wherein a most 5′ pro nucleotide sequence of the two or more pro nucleotide sequences is a part of a prepro nucleotide sequence.

3. The polynucleotide of claim 2, wherein the prepro nucleotide sequence is a cecropin prepro nucleotide sequence.

4. The polynucleotide of claim 2, wherein the prepro nucleotide sequence comprises a sequence shown in SEQ ID NO:3.

5. The polynucleotide of claim 2, wherein the prepro nucleotide sequence comprises a sequence shown in SEQ ID NO:4.

6. The polynucleotide of claim 1, wherein the two or more pro nucleotide sequences each comprise a cecropin pro nucleotide sequence.

7. The polynucleotide of claim 1, wherein the two or more pro nucleotide sequences each comprise a sequence shown in SEQ ID NO:1.

8. The polynucleotide of claim 1, wherein the two or more pro nucleotide sequences each comprise a sequence shown in SEQ ID NO:2.

9. The polynucleotide of claim 1, wherein two genes of interest and two pro nucleotide sequences are arranged in the following order: a prepro nucleotide sequence, a first gene of interest, a pro nucleotide sequence, and a second gene of interest.

10. The polynucleotide of claim 9, wherein the prepro nucleotide sequence is a cecropin prepro nucleotide sequence and the pro nucleotide sequence is a cecropin pro nucleotide sequence.

11. The polynucleotide of claim 9, wherein the prepro nucleotide sequence comprises a sequence shown in SEQ ID NO:3 or SEQ ID NO:4 and the pro nucleotide sequence comprises a sequence shown in SEQ ID NO:1 or SEQ ID NO:2.

12. The polynucleotide of claim 1, wherein a first gene of interest encodes for an antibody heavy chain and a second gene of interest encodes for an antibody light chain.

13. A method of producing a multimeric protein in an individual comprising administering to the individual a polynucleotide comprising two or more genes of interest, wherein each gene of interest encodes a part of the multimeric protein, each gene of interest is operably-linked to a pro nucleotide sequence, and each of the two or more genes of interest may be the same or different.

14. The method of claim 13, wherein the multimeric protein is an associated multimeric protein.

15. The method of claim 13, wherein the multimeric protein is a multivalent multimeric protein.

16. The method of claim 13, wherein the pro nucleotide sequence comprises a cecropin pro nucleotide sequence.

17. The method of claim 13, wherein the pro nucleotide sequence comprises a sequence shown in SEQ ID NO:1.

18. The method of claim 13, wherein the pro nucleotide sequence comprises a sequence shown in SEQ ID NO:2.

19. The method of claim 13, wherein a most 5′ pro nucleotide sequence of the two or more pro sequences is a part of a prepro nucleotide sequence.

20. The method of claim 19, wherein the prepro nucleotide sequence is a cecropin prepro nucleotide sequence.

21. The method of claim 19, wherein the prepro nucleotide sequence comprises a sequence shown in SEQ ID NO:3.

22. The method of claim 19, wherein the prepro nucleotide sequence comprises a sequence shown in SEQ ID NO:4.

23. The method of claim 19, wherein the polynucleotide comprises two genes of interest and two pro nucleotide sequences arranged in the following order: a prepro nucleotide sequence, a first gene of interest, a pro nucleotide sequence, and a second gene of interest.

24. The method of claim 13, wherein a first gene of interest encodes for an antibody heavy chain and a second gene of interest encodes for an antibody light chain.

25. A method of producing a protein in an individual comprising administering to the individual a polynucleotide comprising a cecropin prepro nucleotide sequence operably-linked to one or more genes of interest that encode the protein.

26. The method of claim 25, wherein the prepro nucleotide sequence comprises a sequence shown in SEQ ID NO:3.

27. The method of claim 25, wherein the prepro nucleotide sequence comprises a sequence shown in SEQ ID NO:4.

28. The method of claim 25, wherein a first gene of interest is an antibody heavy chain and a second gene of interest is an antibody light chain.

29. The method of claim 25, wherein the protein is a multimeric protein and the cecropin prepro nucleotide sequence is operably-linked to two or more genes of interest, wherein each gene of interest encodes a part of the multimeric protein.

30. The method of claim 29, wherein the multimeric protein is an associated multimeric protein.

31. The method of claim 29, wherein the multimeric protein is a multivalent multimeric protein.

32. A method of producing a multimeric protein in an individual comprising administering to the individual a polynucleotide comprising two or more genes of interest, wherein each gene of interest encodes a part of the multimeric protein and wherein each gene of interest is operably linked to a gene encoding for a cleavage site.

33. The method of claim 32, wherein a transposon-based vector comprises the polynucleotide and further comprises a transposase gene operably linked to a first promoter and wherein;
a) the first promoter comprises a modified Kozak sequence comprising ACCATG;
b) the two or more genes of interest are each operably-linked to one or more additional promoters; and,
c) the two or more genes of interest and their operably-linked promoters are flanked by transposase insertion sequences recognized by a transposase encoded by the transposase gene.

34. The method of claim 32, wherein a transposon-based vector comprises the polynucleotide and further comprises a transposase gene operably linked to a first promoter and an avian optimized polyA sequence, and wherein;
a) the two or more genes of interest are each operably-linked to one or more additional promoters; and,
b) the two or more genes of interest and their operably-linked promoters are flanked by transposase insertion sequences recognized by a transposase encoded by the transposase gene.

35. The method of claim 32, wherein the cleavage site is selected from a protease cleavage site, a photolabile cleavage site, a pH sensitive cleavage site, a chemical cleavage site and a self-splicing cleavage site.

36. An animal comprising the isolated polynucleotide of claim 1.

37. The animal of claim 36, wherein the animal is a bird.

38. An egg produced by the animal of claim 37.

39. The egg of claim 38, wherein the egg comprises a multimeric protein encoded by the isolated polynucleotide.

40. The animal of claim 36, wherein the animal is a mammal.

41. Milk produced by the mammal of claim 40.

42. The milk of claim 41, wherein the milk comprises a multimeric protein encoded by the isolated polynucleotide.

43. A method of producing a multimeric protein comprising:
a) administering to an egg-laying animal a composition comprising the polynucleotide of claim 1; and,
b) permitting the one or more genes of interest to be expressed into the multimeric protein.

44. The method of claim 43, further comprising
a) collecting an egg from the egg-laying animal;
b) harvesting egg white comprising the multimeric protein; and,
c) purifying the multimeric protein.

45. The method of claim 43, wherein the egg-laying animal is a bird.

46. A method of producing a multimeric protein comprising:
a) administering to an intramammary duct system of a mammal a composition comprising the polynucleotide of claim 1, and,
b) permitting the one or more genes of interest to be expressed into the multimeric protein.

47. The method of claim 46, further comprising
a) collecting milk from the mammal, wherein the milk comprises the multimeric protein; and
b) purifying the multimeric protein.


SEQUENCE LISTING (104 sequences)

SEQ ID NO: 1 - DNA, 15 bp, ARTIFICIAL SEQUENCE
  Synthetic
gcgccagagccgaaa

SEQ ID NO: 2 - DNA, 30 bp, ARTIFICIAL SEQUENCE
  Synthetic
gcgccagagccgaaatggaaagtcttcaag

SEQ ID NO: 3 - DNA, 78 bp, ARTIFICIAL SEQUENCE
  Synthetic
aatttctcaaggatatttttcttcgtgttcgctttggttctggctttgtcaacagtttcggctgcgccagagccgaaa

SEQ ID NO: 4 - DNA, 93 bp, ARTIFICIAL SEQUENCE
  Synthetic
aatttctcaaggatatttttcttcgtgttcgctttggttctggctttgtcaacagtttcggctgcgccagagccgaaatggaaagtcttcaag

SEQ ID NO: 5 - DNA, 6 bp, ARTIFICIAL SEQUENCE
  Synthetic
accatg

SEQ ID NO: 6 - DNA, 54 bp, ARTIFICIAL SEQUENCE
  Synthetic
atgctgggcatctggaccctcctacctctggttcttacgtctgttgctagatta

SEQ ID NO: 7 - DNA, 66 bp, ARTIFICIAL SEQUENCE
  Synthetic
atgattcctgccagatttgccggggtgctgcttgctctggccctcattttgccagggaccctttgt

SEQ ID NO: 8 - DNA, 78 bp, ARTIFICIAL SEQUENCE
  Synthetic
atgggcagagcaatggtggccaggctggggctggggctgctgctgctggcactgctcctacccacgcagatttattcc

SEQ ID NO: 9 - DNA, 63 bp, ARTIFICIAL SEQUENCE
  Synthetic
atgaatctatcgaacatttctgcggtaaaagtattaacactggtggttagcgctgccatcgct

SEQ ID NO: 10 - DNA, 390 bp, ARTIFICIAL SEQUENCE
  Synthetic
atgaccatccttttccttactatggttatctcatacttcagttgcatgaaagctgccccgatgaaagaagctagtgtaagaggacatggcagcttggcttacccaggtcttcggacccacgggactcttgaaagcctaactgggcccaatgctggttcaagaggactgacatcactggcggacacttttgaacacgtgatagaggagcttctagatgaagatcaggacatccagcccagtgaggaaaacaaggatgcggacttgtacacatcccgagtcatgctgagcagtcaagtgcctttggaacccccactgctctttctgctcgaggagtacaaaaactacctggatgctgcaaacatgtccatgagagtccggcgtcactctgac

SEQ ID NO: 11 - DNA, 144 bp, ARTIFICIAL SEQUENCE
  Synthetic
cgtctttttctcttatcttttctcgctttcgctcttttctcgtcggcgattgctttctccgacgacgatccgttgatccgacaagttgtatcgggaaacgatgacaaccatatgttaaacgccgagcatcacttttcacttttt

SEQ ID NO: 12 - DNA, 415 bp, ARTIFICIAL SEQUENCE
  Synthetic
atgtccatcttgttttatgtgatatttcttgcatatcttcgtggcattcagtcaactaatatggatcaaaggagtttgccagaagattcaatgaattctctcattattaaactcattcgggcagacatcttgaaaaacaagctttctaagcaggtgatggatgtcaaggaaaactatcaaaacatagtgcagaaagtagaggaccaccaggagatggatggagatgaaaatgtgaaatcagacttccagccagttatttcaatggatacagacctcctaaggcagcagagacgctacaactctcctcgagttctcctaagtgacaacacaccactggaaccaccaccactgtacctcacagaggattatgttggaagttcagtggtattaaacagaacctctcgaaggaaaaggt

SEQ ID NO: 13 - DNA, 576 bp, ARTIFICIAL SEQUENCE
  Synthetic
atggtgcatctgactcctgaggagaagtctgccgttactgccctgtggggcaaggtgaacgtggatgaagttggtggtgaggccctgggcaggctgctggtggtctacccttggacccagaggttctttgagtcctttggggatctgtccactcctgatgctgttatgggcaaccctaaggtgaaggctcatggcaagaaagtgctcggtgcctttagtgatggcctggctcacctggacaacctcaagggcacctttgccacactgagtgagctgcactgtgacaagctgcacgtggatcctgagaacttcaggctcctgggcaacgtgctggtctgtgtgctggcccatcactttggcaaagaattcaccccaccagtgcaggctgcctatcagaaagtggtggctggtgtggctaatgccctggcccacaagtatcactaagctcgctttcttgctgtccaatttctattaaaggttcctttgttccctaagtccaactactaaactgggggatattatgaagggccttgagcatctggattctgcctaataaaaaacatttattttcattgc

SEQ ID NO: 14 - DNA, 92 bp, ARTIFICIAL SEQUENCE
  Synthetic
gcatggggacggcgcttctccagcgcgggggctgctttctcctgtgcctttcgctgctgctcctgggctgctgggcggagctgggcagcggg

SEQ ID NO: 15 - DNA, 75 bp, ARTIFICIAL SEQUENCE
  Synthetic
cgaaacgattcaaaacctctttactgccgttatttgctggatttttattgctgttttatttggttctggcaggac

SEQ ID NO: 16 - DNA, 377 bp, ARTIFICIAL SEQUENCE
  Synthetic
ggagtctgggggaggcttagtgcagcctggagagtccctgaaactctcctgtgaatccaatgaatacgaattcccttcccatgacatgtcttgggtccgcaagactccggagaagaggctggagttggtcgcagccattaatagtgatggtggtagcacctactatccagacaccatggagagacgattcatcatctccagagacaataccaagaagaccctgtacctgcaaatgagcagtctgaggtctgaggacacagccttgtattactgtgcaagacacacgatgagcaaaagttactgtgagctcaaactaaaacctcctgcagagcatccaggaccagcagggggcgcggagagacacagagttgtgaaat

SEQ ID NO: 17 - DNA, 310 bp, ARTIFICIAL SEQUENCE
  Synthetic
acatccattcttctgtgagtttcactcgaagagcagcgtgtcactgcggacaagccagccagctcaccatggctggacctcccagggtaccagacctctgggaactggccctgagcctcacttcacggacacaggctgcccgccaaagtgggtctcagagcaacagtgtgtgcattgctcgtcacatcttcctcttgctttgcatgactgactacacccaagaagtgtgcccctgggaggaaagcatatttggcaaccagatcataataaaatcagaaatgcagcaaacctttaaaatatccagacttgg

SEQ ID NO: 18 - DNA, 31 bp, ARTIFICIAL SEQUENCE
  Synthetic
tggaagcaagagggagtatgctaacttcatg

SEQ ID NO: 19 - DNA, 16 bp, ARTIFICIAL SEQUENCE
  Synthetic
atcaattacaagaggg

SEQ ID NO: 20 - DNA, 69 bp, ARTIFICIAL SEQUENCE
  Synthetic
atgaagttmgcatactccctcttgcttccattggcaggagtcagtgcttcagtkatcaattacaagaga

SEQ ID NO: 21 - DNA, 39 bp, ARTIFICIAL SEQUENCE
  Synthetic
aattcttaattaattattgtggtgtcacaataacttttc

SEQ ID NO: 22 - DNA, 96 bp, ARTIFICIAL SEQUENCE
  Synthetic
ccccccggatccatggccgctaaattcgtcgtggttctggccgcttgcgtcgccctgagccactcggctatggtgcgccgcaagaagaacggctac

SEQ ID NO: 23 - DNA, 60 bp, ARTIFICIAL SEQUENCE
  Synthetic
ccccccggatccatgaaactcctggtcgtgttcgccatgtgcgtgcccgctgccagcgct

SEQ ID NO: 24 - DNA, 38 bp, ARTIFICIAL SEQUENCE
  Synthetic
cagtgtacggcggctcgaggcagaagtccggacgcata

SEQ ID NO: 25 - DNA, 289 bp, ARTIFICIAL SEQUENCE
  Synthetic
gaattcattatcagcacggaccagatttctggatcaggatagaagtctgacgtttacggttttcgaagcacagaacgcattcgttagcgtaagtgtcaccgtcggtaccgcaaaccggacggtattccagagtgcaaccgttcagttcgttgtagcatttagcttcacgacccagagagtccatggatcccccttccgctgtcttctcagttccaagcattgcgattttgttaagcaacgcactctcgattcgtagagcctcgttgcgtttgtttgcacgaaccatatg

SEQ ID NO: 26 - DNA, 50 bp, ARTIFICIAL SEQUENCE
  Synthetic
ttcacaggcaggtttttgtagagaggggcatgtcatagtcctcactgtgg

SEQ ID NO: 27 - DNA, 309 bp, ARTIFICIAL SEQUENCE
  Synthetic
aagcttctcgtgaaaaccaacccaattagttagtattgcattctgtgtactatagtttggaatattaaaaatattttaaaatacctccattttgcttatccttttagtgaagatgatacctgcaaaagacatggctaaagttatgattgtcatgttggcaatttgttttcttacaaaatcggatgggaaatctgttaagtaagtactgttttgccttggaattggatttttaatgttgactttatcatttcgaagtggggagctaatgggaagtggccctctctgtttctcttcttcccaggaagagat

SEQ ID NO: 28 - DNA, 78 bp, ARTIFICIAL SEQUENCE
  Synthetic
atggctacaggctcccggacgtccctgctcctggcttttggcctgctctgcctgccctggcttcaagagggcagtgcc

SEQ ID NO: 29 - DNA, 54 bp, ARTIFICIAL SEQUENCE
  Synthetic
atgaggtctttgctaatcttggtgctttgcttcctgcccctggctgctctgggg

SEQ ID NO: 30 - DNA, 96 bp, ARTIFICIAL SEQUENCE
  Synthetic
atgcacctgagaatccacgcgagacggaaccctcctcgccggccggcctggacgcttgggatctggtcccttttctggggatgtatcgtcagctct

SEQ ID NO: 31 - DNA, 150 bp, ARTIFICIAL SEQUENCE
  Synthetic
atggccattagtggagtccctgtgctaggatttttcatcatagctgtgctgatgagcgctcaggaatcatgggctatcaaagaagaacatgtgatcatccaggccgagttctatctgaatcctgaccaatcaggcgagtttatgtttgac

SEQ ID NO: 32 - DNA, 79 bp, ARTIFICIAL SEQUENCE
  Synthetic
aggggggatccccggagaccttcgggtagcaactgtcaccttgatgctggcgatcctgagctcctcactggctgagggc

SEQ ID NO: 33 - DNA, 87 bp, ARTIFICIAL SEQUENCE
  Synthetic
atggtgtgtctgaggctccctggaggctcctgcatggcagttctgacagtgacactgatggtgctgagctccccactggctttggct

SEQ ID NO: 34 - DNA, 4045 bp, ARTIFICIAL SEQUENCE
  Synthetic
gaacgatttaaggagcgaatactactggtaaactaatggaagaaatctgctgcaccactggatattgggagtgtgtggcatgcatcctcatcatcaggaaactctaaaaaagaaccgagtggtgctagccaaacagctgttgttgagcgaattgttagaacatcttctggagaaggacatcatcaccttggaaatgagggagctcatccaggccaaagtgggcagtttcagccagaatgtggaactcctcaacttgctgcctaagaggggtccccaagcttttgatgccttctgtgaagcactgagggagaccaagcaaggccacctggaggatatgttgctcaccaccctttctgggcttcagcatgtactcccaccgttgagctgtgactacgacttgagtctcccttttccggtgtgtgagtcctgtcccctttacaagaagctccgcctgtcgacagatactgtggaacactccctagacaataaagatggtcctgtctgccttcaggtgaagccttgcactcctgaattttatcaaacacacttccagctggcatataggttgcagtctcggcctcgtggcctagcactggtgttgagcaatgtgcacttcactggagagaaagaactggaatttcgctctggaggggatgtggaccacagtactctagtcaccctcttcaagcttttgggctatgacgtccatgttctatgtgaccagactgcacaggaaatgcaagagaaactgcagaattttgcacagttacctgcacaccgagtcacggactcctgcatcgtggcactcctctcgcatggtgtggagggcgccatctatggtgtggatgggaaactgctccagctccaagaggtttttcagctctttgacaacgccaactgcccaagcctacagaacaaaccaaaaatgttcttcatccaggcctgccgtggaggtgctattggatcccttgggcacctccttctgttcactgctgccaccgcctctcttgctctatgagactgatcgtggggttgaccaacaagatggaaagaaccacgcaggatcccctgggtgcgaggagagtgatgccggtaaagaaaagttgccgaagatgagactgcccacgcgctcagacatgatatgcggctatgcctgcctcaaagggactgccgccatgcggaacaccaaacgaggttcctggtacatcgaggctcttgctcaagtgttttctgagcgggcttgtgatatgcacgtggccgacatgctggttaaggtgaacgcacttatcaaggatcgggaaggttatgctcctggcacagaattccaccggtgcaaggagatgtctgaatactgcagcactctgtgccgccacctctacctgttcccaggacaccctcccacatgatgtcacctccccatcatccacgccaagtggaagccactggaccacaggaggtgtgatagagcctttgatcttcaggatgcacggtttctgttctgccccctcagggatgtgggaatctcccagacttgtttcctgtgcccatcatctctgcctttgagtgtgggactccaggccagctccttttctgtgaagccctttgcctgtagagccagccttggttggacctattgccaggaatgtttcagctgcagttgaagagcctgacaagtgaagttgtaaacacagtgtggttatggggagagggcatataaattccccatatttgtgttcagttccagcttttgtagatggcactttagtgattgcttttattacattagttaagatgtctgagagaccatctcctatcttttatttcattcatatcctccgccctttttgtcctagagtgagagtttggaaggtgtccaaatttaatgtagacattatcttttggctctgaagaagcaaacatgactagagacgcaccttgctgcagtgtccagaagcggcctgtgcgttcccttcagtactgcagcgccacccagtggaaggacactcttggctcgtttgggctcaaggcaccgcagcctgtcagccaacattgccttgcatttgtaccttattgatctttgcccatggaagtctcaaagatctttcgttggttgtttctctgagctttgttactgaaatgagcctcgtggggagcatcagagaaggccaggaagaatggtgtgtttccctagactctgtaaccacctctctgtctttttccttcctgagaaacgtccatctctctcccttactattcccactttcattcaatcaacctgcacttcatatctagatttctagaaaagcttcctagcttatctccctgcttcatatctctcccttctttaccttcatttcatcctgttggctgctgccaccaaatctgtctagaatcctgctttacaggatcatgtaaatgctcaaagatgtaatgtagttctttgttcctgctttctctttcagtattaaactctcctttgatattatgtggcttttatttcagtgccatacatgttattgttttcaacctagaaacctttatccctgcttatctgaaacttcccaacttccctgttctttaagactttttttttttttttttttttttttgagacagagtctcgctctgtcgcccaggctggagggcagtggcacgatctcagctcactgcaagctccaactcccgggttcacgccattctcctgcctcagccttccaagtagctgggactacaggtgcccgccaccgtgcccggctaatttttttgtatttttagtagagacagggtttcaccatgttagccgggatggtcttgatctcctgacctcatgatccacccacctcagcctcccaaagtgttgggattacaggcgtgagccactgcgcccgggcaagacctttttttaaaaaaaaaaaaaaaaaaacttccattctttcttcctccagtctgttctcacataacagagtagttttggtttttaattttttttggttgtttgctgttttttgttttttaaggtgagttctcactatgtttctcagactggtctcgaactcctggcctcaagccatcttcccgcctcagcctctcaaatagctgggcttacaggcatgagccaccacacctggccaggatttggttgtttaaatataaatctgatcacccccctgcttagaacccttctgctttctattacccctcatttaaaatgtaaactcttcaccttggtttatgagaactggttcttgccttccccttgaacctcattaaatggtgatttcttgctaagctccagcccgagtggtctcctctcagcttctaattttgtgctctttcctgcccttttcctgggccttctcagctctccacccccaccactcttgactcaggtggtgtccttcttcctcaagtcttgacaattcccgggcccttcagtccctgagcagtctacttctgtgtctgtcaccacatcttgtcttttcccctcattgcatttattgcagtttatatatatgctacttttacttgttcatttctgtctcccctaccaggctgtaaatgagggcagaaaccttgtttgttttattcaccatcatgtaccaagtgcttggcacatagtgggccttcattaaatgtttgttgaataaaagagggaagaaggcaagccaaccttagctacaatcctaccttttgataaaatgttccttttgacaatatacacggattattatttgtactttgtttttccatgtgttttgcttttatccactggcatttttagctccttgaagacatatcatgtgtgagataacttccttcacatctcccatggtccctagcaaaatgctaggcctgtagtagtcaaggtgctcaataaatatttgtttgggtggtttgtgagccttgctgccaagtcctgcctttgggtcgacatagtatggaagtatttgagagagagaacctttccactcccactgccaggattttgtattgccatcgggtgccaaataaatgctcatatttattaaaaaaaaaaaaaaaaaaa

SEQ ID NO: 35 - DNA, 501 bp, ARTIFICIAL SEQUENCE
  Synthetic
tccagatcatctgtcctcaccaccaaggccatggtgtcttcagcgactatctgcagtttgctactcctcagcatgctctggatggacatggccatggcaggttccagcttcttgagcccagagcaccagaaagcccagcagagaaaggaatccaagaagccaccagctaaactgcagccacgagctctggaaggctggctccacccagaggacagaggacaagcagaagaggcagaggaggagctggaaatcaggttcaatgctcccttcgatgttggcatcaagctgtcaggagctcagtaccagcagcatggccgggccctgggaaagtttcttcaggatatcctctgggaagaggtcaaagaggcgccagctaacaagtaaccactgacaggactggtccctgtactttcctcctaagcaagaactcacatccagcttctgcctcctctgcaactcccagcactctcctgctgacttacaaataaatgttcaagctgt

SEQ ID NO: 36 - DNA, 70 bp, ARTIFICIAL SEQUENCE
  Synthetic
atgaagctgcttgcaatggttgcactgctggtcaccatctgtagcctagaaggagctttggttcggagac

SEQ ID NO: 37 - DNA, 70 bp, ARTIFICIAL SEQUENCE; identical to SEQ ID NO: 36
  Synthetic

SEQ ID NO: 38 - DNA, 99 bp, ARTIFICIAL SEQUENCE
  Synthetic
atggccttgccaacggctcgacccctgttggggtcctgtgggacccccgccctcggcagcctcctgttcctgctcttcagcctcggatgggtgcagccc

SEQ ID NO: 39 - DNA, 72 bp, ARTIFICIAL SEQUENCE
  Synthetic
atgccgcgcctgttctcctacctcctaggtgtctggctgctcctgagccaacttcccagagaaatcccaggc

SEQ ID NO: 40 - DNA, 75 bp, ARTIFICIAL SEQUENCE
  Synthetic
atgacagcatcacttgtcgttttaccatcgctttggttaatattaattatttttactgcaccctatactcactgt

SEQ ID NO: 41 - DNA, 388 bp, ARTIFICIAL SEQUENCE
  Synthetic
atgaaagtcctgctttgtgacctgctgctgctcagtctcttctccagtgtgttcagcagttgtcagagggactgtctcacatgccaggagaagctccacccagccctggacagcttcgacctggaggtgtgcatcctcgagtgcgaagagaaggtcttccccagccccctctggactccatgcaccaaggtcatggccaggagctcttggcagctcagccctgccgccccagagcatgtggcggctgctctctaccagccgagagcttcggagatgcagcatctgcggcgaatgccccgagtccggagcttgttccaggagcaggaagagcccgagcctggcatggaggaggctggtgagatggagcagaagcagctgcagaagagat

SEQ ID NO: 42 - DNA, 76 bp, ARTIFICIAL SEQUENCE
  Synthetic
aattcatgaagtgggttactttcatctctttgttgttcttgttctcttctgcttactctagaggtgttttcagacg

SEQ ID NO: 43 - DNA, 72 bp, ARTIFICIAL SEQUENCE
  Synthetic
atgaagtgggttactttcatctctttgttgttcttgttctcttctgcttactctagaggtgttttcagacgc

SEQ ID NO: 44 - DNA, 1731 bp, ARTIFICIAL SEQUENCE
  Synthetic
gaattctcaatggcaaaggcaagtgtacattataaatagcaaaacagctggcttggaccatgttgccggccagtcacccagttgagggatttgaatgacatcataaccctcaagagggtattgctagccagctggtgttatttagaatacacaaaaatcagagaaagaaaacacactctggcacacagactccctctgtcatacacacacacacacacacacacacacacacacacacacagaggtttgagttatatggaaaattcaaacaacaggaaaattgtttgccccccaggtacccttctcccagagtggtggggtggggaggggacagtgacaggcagcctagtagaagaataaagaaaaatgttctatttcagttgggttttacagctcggcatagtctttgcctcatcgcaggagaaaaagtatgagacagtgccctaaagggaccaatccaatgctgcctgcccctccatacgttctaggaaatgagatcacacccctcacttggcaactgggacaaggggtcacccgagtgctgtcttccaatctactttaccccagtcacttcagggttaaaattgtagagtttgctggagagggtcttatcgtcctttctttctttttttgttttaaataatgcatttgctctagaatctaaaattgctctcccatcccccatattcctttaatactggtaaggtgtattagcagacgtttgtgtcttcatgcccagcagaaagttaatcagaaaacagatccttattttctatggcagcataagtattttaatgtctgcgaaccctgtcagtaacacacattcttttaagggaaaaaaatgcttctgtgctctagttttaaaatgcaaaggtatgatgttatttgtcaccatgcccaaaaaagtccttactcaataactttgccagaagagggagagagagagaaggcaaatgttcccccagctgtttcctgtctacagtgtctgtgttttgtagataaatgtgaggattttgtgtaaatccctcttctgtttgctaaatctcactgtcactgctaaattcagagcagatagagcctgcgcaatggaataaagtcctcaaaattgaaatgtgacattgctctcaacatctcccatctctctggatttctttttgcttcattattcctgctaaccaattcattttcagactttgtacttcagaagcaatgggaaaaatcagcagtcttccaacccaattatttaagtgctgcttttgtgatttcttgaaggtaaatatttcttactctttgaagtcattggggaattcggatcccactgtaataatagcatctttcatttccgtagtaaacgtttctagatattttgtctcaattcattgaaataggaacccataaagaaaggggttcagggaggactcctccaaagatccacagtagccaggggaataaacacaggttgttggatgccgagacacgctccatccacaactccctgctgggttctcatgtactctattggcttctgtgctgggtagtcctgattaatgacagtcgtggaatcgtgggagtcaatgcacttctgtcccaccccactccccttgcaaggatcaaggaggaaacctgaacctccctctgtttcttgggcaggtgaagatgcacaccatgtcctcctcgcatctcttctacctggcgctgtgcctgctcaccttcaccagctctgccacggct

SEQ ID NO: 45 - DNA, 122 bp, ARTIFICIAL SEQUENCE
  Synthetic
atgaagccaattcaaaaactcctagctggccttattctactgacttcgtgcgtggaaggctgctccagccagcactggtcctatggactgcgccctggaggaaagagagatgccgaaaattt

SEQ ID NO: 46 - DNA, 268 bp, ARTIFICIAL SEQUENCE
  Synthetic
atgagatttccttcaatttttactgcagttttattcgcagcatcctccgcattagctgctccagtcaacactacaacagaagatgaaacggcacaaattccggctgaagctgtcatcggttacttagatttagaaggggatttcgatgttgctgttttgccattttccaacagcacaaataacgggttattgtttataaatactactattgccagcattgctgctaaagaagaaggggtatctttggataaaagagaggctgaagctt

SEQ ID NO: 47 - DNA, 74 bp, ARTIFICIAL SEQUENCE
  Synthetic
atgaagtgggtaacctttatttcccttctttttctctttagctcggcttattccaggggtgtgtttcgtcgaga

SEQ ID NO: 48 - DNA, 366 bp, ARTIFICIAL SEQUENCE
  Synthetic
atgaagatggtctcctcctcgcgcctccgctgcctcctcgtgctcctgctgtccctgaccgcctccatcagctgctccttcgccggacagagagactccaaactccgcctgctgctgcaccggtacccgctgcagggctccaaacaggacatgactcgctccgccttggccgagctgctcctgtcggacctcctgcagggggagaacgaggctctggaggaggagaacttccctctggccgaaggaggacccgaggacgcccacgccgacctagagcgggccgccagcggggggcctctgctcgccccccgggagagaaaggccggctgcaagaacttcttctggaaaaccttcacctcctgctga

SEQ ID NO: 49 - DNA, 1428 bp, ARTIFICIAL SEQUENCE
  Synthetic
atggccgggcgagggggcagcgcgctgctggctctgtgcggggcactggctgcctgcgggtggctcctgggcgccgaagcccaggagcccggggcgcccgcggcgggcatgaggcggcgccggcggctgcagcaagaggacggcatctccttcgagtaccaccgctaccccgagctgcgcgaggcgctcgtgtccgtgtggctgcagtgcaccgccatcagcaggatttacacggtggggcgcagcttcgagggccgggagctcctggtcatcgagctgtccgacaaccctggcgtccatgagcctggtgagcctgaatttaaatacattgggaatatgcatgggaatgaggctgttggacgagaactgctcattttcttggcccagtacctatgcaacgaataccagaaggggaacgagacaattgtcaacctgatccacagtacccgcattcacatcatgccttccctgaacccagatggctttgagaaggcagcgtctcagcctggtgaactcaaggactggtttgtgggtcgaagcaatgcccagggaatagatctgaaccggaactttccagacctggataggatagtgtacgtgaatgagaaagaaggtggtccaaataatcatctgttgaaaaatatgaagaaaattgtggatcaaaacacaaagcttgctcctgagaccaaggctgtcattcattggattatggatattccttttgtgctttctgccaatctccatggaggagaccttgtggccaattatccatatgatgagacgcggagtggtagtgctcacgaatacagctcctccccagatgacgccattttccaaagcttggcccgggcatactcttctttcaacccggccatgtctgaccccaatcggccaccatgtcgcaagaatgatgatgacagcagctttgtagatggaaccaccaacggtggtgcttggtacagcgtacctggagggatgcaagacttcaattaccttagcagcaactgttttgagatcaccgtggagcttagctgtgagaagttcccacctgaagagactctgaagacctactgggaggataacaaaaactccctcattagctaccttgagcagatacaccgaggagttaaaggatttgtccgagaccttcaaggtaacccaattgcgaatgccaccatctccgtggaaggaatagaccacgatgttacatccgcaaaggatggtgattactggagattgcttatacctggaaactataaacttacagcctcagctccaggctatctggcaataacaaagaaagtggcagttccttacagccctgctgctggggttgattttgaactggagtcattttctgaaaggaaagaagaggagaaggaagaattgatggaatggtggaaaatgatgtcagaaactttaaatttt

SEQ ID NO: 50 - DNA, 69 bp, ARTIFICIAL SEQUENCE
  Synthetic
atggctctctcactcttcactgttggacaattaattttcttattttggacactcagaatcactgaagcc

SEQ ID NO: 51 - DNA, 106 bp, ARTIFICIAL SEQUENCE
  Synthetic
atgaacaaactagcaattctcgctatcatcgctatggtacttttcagcgcaaacgccttcagactccaaagcagattgagatcaaatatggaagcttctgccagag

SEQ ID NO: 52 - DNA, 261 bp, ARTIFICIAL SEQUENCE
  Synthetic
atggtcagtgtgtgcaggctcttgctggttgctgccttgctgctgtgtttgcaagcacagctgtctttctctcagcactggtctcatggctggtaccctggaggaaagagagaaatcgactcctacagctcaccagagatatctggggagattaaactgtgtgaagcgggagaatgcagctatctcaggccactgaggaccaacatcctaaagagcatcctgattgacacccttgcaaggaaattccaaaagaggaaatga

SEQ ID NO: 53 - DNA, 304 bp, ARTIFICIAL SEQUENCE
  Synthetic
tgtgttttgtagataaatgtgaggattttctctaaatccctcttctgcttgctaaatctcactgtcgctgctaaattcagagcagatagagcctgcgcaatcgaaataaagtcctcaaaattgaaatgtgactttgctctaacatctcccatctctctggatttctttttgcctcattattcctgcccaccaattcatttccagactttgtacttcagaagcgatggggaaaatcagcagtcttccaactcaattatttaagatctgcctctgtgacttcttgaagataaagatacacatcatg

SEQ ID NO: 54 - DNA, 81 bp, ARTIFICIAL SEQUENCE
  Synthetic
atgtcaggcccgaggacgtgcttctgtctaccgtcggctcttgtactagtactgctgagtctcagcacttcggcactaggg

SEQ ID NO: 55 - protein, 474 aa, ARTIFICIAL SEQUENCE
  Synthetic
MSPAAQLAKAAARSTCMTRLPSGIRVATAPSNSHFAAVGVYVDAGPIYETSIDRGVSHFVSSLAFKSTHGATESQVLKTMAGLGGNLFCTATRESILYQGSVLHHDLPRTVQLLADTTLRPALTEEEIAERRATIAFEAEDLHSRPDAFIGEMMHAVAFGGRGLGNSIFCEPQRARNMTSDTIREYFATYLHPSRMVVAGTGVAHAELVDLVSKAFVPSSTRAPSSVTHSDIETAYVGGSHQLVIPKPPPTHPNYEQTLTHVQVAFPVPPFTHPDMFPVSTLQVLMGGGGAFSAGGPGKGMYSRLYTNVLNRYRWMESCAAFQHAYSSTSLFGISASCVPSFNPHLCNVLAGEFVHMARNLSDEEVARAKNQLKSSLLMNLESQVITVEDIGRQVLAQNQRLEPLELVNNISAVTRDDLVRVAEALVAKPPTMVAVGEDLTKLTDIKETLAAFNASGEALQPVGSAGSFGRVTM

SEQ ID NO: 56 - DNA, 7315 bp, ARTIFICIAL SEQUENCE
  Synthetic
ctgacgcgccctgtagcggcgcattaagcgcggcgggtgtggtggttacgcgcagcgtgaccgctacacttgccagcgccctagcgcccgctcctttcgctttcttcccttcctttctcgccacgttcgccggcatcagattggctattggccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccgcggccgggaacggtgcattggaacgcggattccccgtgccaagagtgacgtaagtaccgcctatagactctataggcacacccctttggctcttatgcatgctatactgtttttggcttggggcctatacacccccgcttccttatgctataggtgatggtatagcttagcctataggtgtgggttattgaccattattgaccactcccctattggtgacgatactttccattactaatccataacatggctctttgccacaactatctctattggctatatgccaatactctgtccttcagagactgacacggactctgtatttttacaggatggggtcccatttattatttacaaattcacatatacaacaacgccgtcccccgtgcccgcagtttttattaaacatagcgtgggatctccacgcgaatctcgggtacgtgttccggacatgggctcttctccggtagcggcggagcttccacatccgagccctggtcccatgcctccagcggctcatggtcgctcggcagctccttgctcctaacagtggaggccagacttaggcacagcacaatgcccaccaccaccagtgtgccgcacaaggccgtggcggtagggtatgtgtctgaaaatgagcgtggagattgggctcgcacggctgacgcagatggaagacttaaggcagcggcagaagaagatgcaggcagctgagttgttgtattctgataagagtcagaggtaactcccgttgcggtgctgttaacggtggagggcagtgtagtctgagcagtactcgttgctgccgcgcgcgccaccagacataatagctgacagactaacagactgttcctttccatgggtcttttctgcagtcaccgtcggaccatgtgcgaactcgatattttacacgactctctttaccaattctgccccgaattacacttaaaacgactcaacagcttaacgttggcttgccacgcattacttgactgtaaaactctcactcttaccgaacttggccgtaacctgccaaccaaagcgagaacaaaacataacatcaaacgaatcgaccgattgttaggtaatcgtcacctccacaaagagcgactcgctgtataccgttggcatgctagctttatctgttcgggcaatacgatgcccattgtacttgttgactggtctgatattcgtgagcaaaaacgacttatggtattgcgagcttcagtcgcactacacggtcgttctgttactctttatgagaaagcgttcccgctttcagagcaatgttcaaagaaagctcatgaccaatttctagccgaccttgcgagcattctaccgagtaacaccacaccgctcattgtcagtgatgctggctttaaagtgccatggtataaatccgttgagaagctgggttggtactggttaagtcgagtaagaggaaaagtacaatatgcagacctaggagcggaaaactggaaacctatcagcaacttacatgatatgtcatctagtcactcaaagactttaggctataagaggctgactaaaagcaatccaatctcatgccaaattctattgtataaatctcgctctaaaggccgaaaaaatcagcgctcgacacggactcattgtcaccacccgtcacctaaaatctactcagcgtcggcaaaggagccatgggttctagcaactaacttacctgttgaaattcgaacacccaaacaacttgttaatatctattcgaagcgaatgcagattgaagaaaccttccgagacttgaaaagtcctgcctacggactaggcctacgccatagccgaacgagcagctcagagcgttttgatatcatgctgctaatcgccctgatgcttcaactaacatgttggcttgcgggcgttcatgctcagaaacaaggttgggacaagcacttccaggctaacacagtcagaaatcgaaacgtactctcaacagttcgcttaggcatggaagttttgcggcattctggctacacaataacaagggaagacttactcgtggctgcaaccctactagctcaaaatttattcacacatggttacgctttggggaaattatgaggggatcgctctagagcgatccgggatctcgggaaaagcgttggtgaccaaaggtgccttttatcatcactttaaaaataaaaaacaattactcagtgcctgttataagcagcaattaattatgattgatgcctacatcacaacaaaaactgatttaacaaatggttggtctgccttagaaagtatatttgaacattatcttgattatattattgataataataaaaaccttatccctatccaagaagtgatgcctatcattggttggaatgaacttgaaaaaaattagccttgaatacattactggtaaggtaaacgccattgtcagcaaattgatccaagagaaccaacttaaagctttcctgacggaatgttaattctcgttgaccctgagcactgatgaatcccctaatgattttggtaaaaatcattaagttaaggtggatacacatcttgtcatatgatcccggtaatgtgagttagctcactcattaggcaccccaggctttacactttatgcttccggctcgtatgttgtgtggaattgtgagcggataacaatttcacacaggaaacagctatgaccatgattacgccaagcgcgcaattaaccctcactaaagggaacaaaagctggagctccaccgcggtggcggccgctctagaactagtggatcccccgggctgcaggaattcgatatcaagcttatcgataccgctgacctcgagggggggcccggtacccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactactccgggatcatatgacaagatgtgtatccaccttaacttaatgatttttaccaaaatcattaggggattcatcagtgctcagggtcaacgagaattaacattccgtcaggaaagcttatgatgatgatgtgcttaaaaacttactcaatggctggttatgcatatcgcaatacatgcgaaaaacctaaaagagcttgccgataaaaaaggccaatttattgctatttaccgcggctttttattgagcttgaaagataaataaaatagataggttttatttgaagctaaatcttctttatcgtaaaaaatgccctcttgggttatcaagagggtcattatatttcgcggaataacatcatttggtgacgaaataactaagcacttgtctcctgtttactcccctgagcttgaggggttaacatgaaggtcatcgatagcaggataataatacagtaaaacgctaaaccaataatccaaatccagccatcccaaattggtagtgaatgattataaataacagcaaacagtaatgggccaataacaccggttgcattggtaaggctcaccaataatccctgtaaagcaccttgctgatgactctttgtttggatagacatcactccctgtaatgcaggtaaagcgatcccaccaccagccaataaaattaaaacagggaaaactaaccaaccttcagatataaacgctaaaaaggcaaatgcactactatctgcaataaatccgagcagtactgccgttttttcgcccatttagtggctattcttcctgccacaaaggcttggaatactgagtgtaaaagaccaagacccgtaatgaaaagccaaccatcatgctattcatcatcacgatttctgtaatagcaccacaccgtgctggattggctatcaatgcgctgaaataataatcaacaaatggcatcgttaaataagtgatgtataccgatcagcttttgttccctttagtgagggttaattgcgcgcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccac

SEQ ID NO: 57 - DNA, 7689 bp, ARTIFICIAL SEQUENCE
  Synthetic
ctgacgcgccctgtagcggcgcattaagcgcggcgggtgtggtggttacgcgcagcgtgaccgctacacttgccagcgccctagcgcccgctcctttcgctttcttcccttcctttctcgccacgttcgccggcatcagattggctattggccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccgcggccgggaacggtgcattggaacgcggattccccgtgccaagagtgacgtaagtaccgcctatagactctataggcacacccctttggctcttatgcatgctatactgtttttggcttggggcctatacacccccgcttccttatgctataggtgatggtatagcttagcctataggtgtgggttattgaccattattgaccactcccctattggtgacgatactttccattactaatccataacatggctctttgccacaactatctctattggctatatgccaatactctgtccttcagagactgacacggactctgtatttttacaggatggggtcccatttattatttacaaattcacatatacaacaacgccgtcccccgtgcccgcagtttttattaaacatagcgtgggatctccacgcgaatctcgggtacgtgttccggacatgggctcttctccggtagcggcggagcttccacatccgagccctggtcccatgcctccagcggctcatggtcgctcggcagctccttgctcctaacagtggaggccagacttaggcacagcacaatgcccaccaccaccagtgtgccgcacaaggccgtggcggtagggtatgtgtctgaaaatgagcgtggagattgggctcgcacggctgacgcagatggaagacttaaggcagcggcagaagaagatgcaggcagctgagttgttgtattctgataagagtcagaggtaactcccgttgcggtgctgttaacggtggagggcagtgtagtctgagcagtactcgttgctgccgcgcgcgccaccagacataatagctgacagactaacagactgttcctttccatgggtcttttctgcagtcaccgtcggaccatgtgtgaacttgatattttacatgattctctttaccaattctgccccgaattacacttaaaacgactcaacagcttaacgttggcttgccacgcattacttgactgtaaaactctcactcttaccgaacttggccgtaacctgccaaccaaagcgagaacaaaacataacatcaaacgaatcgaccgattgttaggtaatcgtcacctccacaaagagcgactcgctgtataccgttggcatgctagctttatctgttcgggaatacgatgcccattgtacttgttgactggtctgatattcgtgagcaaaaacgacttatggtattgcgagcttcagtcgcactacacggtcgttctgttactctttatgagaaagcgttcccgctttcagagcaatgttcaaagaaagctcatgaccaatttctagccgaccttgcgagcattctaccgagtaacaccacaccgctcattgtcagtgatgctggctttaaagtgccatggtataaatccgttgagaagctgggttggtactggttaagtcgagtaagaggaaaagtacaatatgcagacctaggagcggaaaactggaaacctatcagcaacttacatgatatgtcatctagtcactcaaagactttaggctataagaggctgactaaaagcaatccaatctcatgccaaattctattgtataaatctcgctctaaaggccgaaaaaatcagcgctcgacacggactcattgtcaccacccgtcacctaaaatctactcagcgtcggcaaaggagccatgggttctagcaactaacttacctgttgaaattcgaacacccaaacaacttgttaatatctattcgaagcgaatgcagattgaagaaaccttccgagacttgaaaagtcctgcctacggactaggcctacgccatagccgaacgagcagctcagagcgttttgatatcatgctgctaatcgccctgatgcttcaactaacatgttggcttgcgggcgttcatgctcagaaacaaggttgggacaagcacttccaggctaacacagtcagaaatcgaaacgtactctcaacagttcgcttaggcatggaagttttgcggcattctggctacacaataacaagggaagacttactcgtggctgcaaccctactagctcaaaatttattcacacatggttacgctttggggaaattatgataatgatccagatcacttctggctaataaaagatcagagctctagagatctgtgtgttggttttttgtggatctgctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcagcacagcaagggggaggattgggaagacaatagcaggcatgctggggatgcggtgggctctatgggtacctctctctctctctctctctctctctctctctctctctctcggtacctctctctctctctctctctctctctctctctctctctctcggtaccaggtgctgaagaattgacccggtgaccaaaggtgccttttatcatcactttaaaaataaaaaacaattactcagtgcctgttataagcagcaattaattatgattgatgcctacatcacaacaaaaactgatttaacaaatggttggtctgccttagaaagtatatttgaacattatcttgattatattattgataataataaaaaccttatccctatccaagaagtgatgcctatcattggttggaatgaacttgaaaaaaattagccttgaatacattactggtaaggtaaacgccattgtcagcaaattgatccaagagaaccaacttaaagctttcctgacggaatgttaattctcgttgaccctgagcactgatgaatcccctaatgattttggtaaaaatcattaagttaaggtggatacacatcttgtcatatgatcccggtaatgtgagttagctcactcattaggcaccccaggctttacactttatgcttccggctcgtatgttgtgtggaattgtgagcggataacaatttcacacaggaaacagctatgaccatgattacgccaagcgcgcaattaaccctcactaaagggaacaaaagctggagctccaccgcggtggcggccgctctagaactagtggatcccccgggctgcaggaattcgatatcaagcttatcgataccgctgacctcgagggggggcccggtacccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactactccgggatcatatgacaagatgtgtatccaccttaacttaatgatttttaccaaaatcattaggggattcatcagtgctcagggtcaacgagaattaacattccgtcaggaaagcttatgatgatgatgtgcttaaaaacttactcaatggctggttatgcatatcgcaatacatgcgaaaaacctaaaagagcttgccgataaaaaaggccaatttattgctatttaccgcggctttttattgagcttgaaagataaataaaatagataggttttatttgaagctaaatcttctttatcgtaaaaaatgccctcttgggttatcaagagggtcattatatttcgcggaataacatcatttggtgacgaaataactaagcacttgtctcctgtttactcccctgagcttgaggggttaacatgaaggtcatcgatagcaggataataatacagtaaaacgctaaaccaataatccaaatccagccatcccaaattggtagtgaatgattataaataacagcaaacagtaatgggccaataacaccggttgcattggtaaggctcaccaataatccctgtaaagcaccttgctgatgactctttgtttggatagacatcactccctgtaatgcaggtaaagcgatcccaccaccagccaataaaattaaaacagggaaaactaaccaaccttcagatataaacgctaaaaaggcaaatgcactactatctgcaataaatccgagcagtactgccgttttttcgcccatttagtggctattcttcctgccacaaaggcttggaatactgagtgtaaaagaccaagacccgtaatgaaaagccaaccatcatgctattcatcatcacgatttctgtaatagcaccacaccgtgctggattggctatcaatgcgctgaaataataatcaacaaatggcatcgttaaataagtgatgtataccgatcagcttttgttccctttagtgagggttaattgcgcgcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccac

SEQ ID NO: 58 - DNA, 7 bp, ARTIFICIAL SEQUENCE
  Synthetic
accatgg

SEQ ID NO: 59 - DNA, 7 bp, ARTIFICIAL SEQUENCE
  Synthetic
accatgt

SEQ ID NO: 60 - DNA, 7 bp, ARTIFICIAL SEQUENCE
  Synthetic
aagatgt

SEQ ID NO: 61 - DNA, 7 bp, ARTIFICIAL SEQUENCE
  Synthetic
acgatga

SEQ ID NO: 62 - DNA, 7 bp, ARTIFICIAL SEQUENCE
  Synthetic
aagatgg

SEQ ID NO: 63 - DNA, 7 bp, ARTIFICIAL SEQUENCE
  Synthetic
gacatga

SEQ ID NO: 64 - DNA, 7 bp, ARTIFICIAL SEQUENCE
  Synthetic
accatga

SEQ ID NO: 65 - DNA, 7 bp, ARTIFICIAL SEQUENCE
  Synthetic
accatgt

SEQ ID NO: 66 - DNA, 315 bp, GALLUS SP.
tctgccattgctgcttcctctgcccttcctcgtcactctgaatgtggcttcttcgctactgccacagcaagaaataaaatctcaacatctaaatgggtttcctgaggtttttcaagagtcgttaagcacattccttccccagcaccccttgctgcaggccagtgccaggcaccaacttggctactgctgcccatgagagaaatccagttcaatattttccaaagcaaaatggattacatatgccctagatcctgattaacaggcgtttgtattatctagtgctttcgcttcacccagattatcccattgcctccc

SEQ ID NO: 67 - DNA, 361 bp, ARTIFICIAL SEQUENCE
  Synthetic
ggcgcctggatccagatcacttctggctaataaaagatcagagctctagagatctgtgtgttggttttttgtggatctgctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcagcacagcaagggggaggattgggaagacaatagcaggcatgctggggatgcggtgggctctatgggtacctctctctctctctctctctctctctctctctctctctctcggtacctctctc

SEQ ID NO: 68 - DNA, 350 bp, ARTIFICIAL SEQUENCE
  Synthetic
ggggatcgctctagagcgatccgggatctcgggaaaagcgttggtgaccaaaggtgccttttatcatcactttaaaaataaaaaacaattactcagtgcctgttataagcagcaattaattatgattgatgcctacatcacaacaaaaactgatttaacaaatggttggtctgccttagaaagtatatttgaacattatcttgattatattattgataataataaaaaccttatccctatccaagaagtgatgcctatcattggttggaatgaacttgaaaaaaattagccttgaatacattactggtaaggtaaacgccattgtcagcaaattgatccaagagaaccaa

SEQ ID NO: 69 - DNA, 908 bp, ARTIFICIAL SEQUENCE
  Synthetic
tgaatgtgttcttgtgttatcaatataaatcacagttagtgatgaagttggctgcaagcctgcatcagttcagctacttggctgcattttgtatttggttctgtaggaaatgcaaaaggttctaggctgacctgcacttctatccctcttgccttactgctgagaatctctgcaggttttaattgttcacattttgctcccatttactttggaagataaaatatttacagaatgcttatgaaacctttgttcatttaaaaatattcctggtcagcgtgaccggagctgaaagaacacattgatcccgtgatttcaataaatacatatgttccatatattgtttctcagtagcctcttaaatcatgtgcgttggtgcacatatgaatacatgaatagcaaaggtttatctggattacgctctggcctgcaggaatggccataaaccaaagctgagggaagagggagagtatagtcaatgtagattatactgattgctgattgggttattatcagctagataacaacttgggtcaggtgccaggtcaacataacctgggcaaaaccagtctcatctgtggcaggaccatgtaccagcagccagccgtgacccaatctaggaaagcaagtagcacatcaattttaaatttattgtaaatgccgtagtagaagtgttttactgtgatacattgaaacttctggtcaatcagaaaaaggttttttatcagagatgccaaggtattatttgattttctttattcgccgtgaagagaatttatgattgcaaaaagaggagtgtttacataaactgataaaaaacttgaggaattcagcagaaaacagccacgtgttcctgaacattcttccataaaagtctcaccatgcctggcagagccctattcaccttcgct

SEQ ID NO: 70 - DNA, 901 bp, Gallus
gaggtcagaatggtttctttactgtttgtcaattctattatttcaatacagaacaatagcttctataactgaaatatatttgctattgtatattatgattgtccctcgaaccatgaacactcctccagctgaatttcacaattcctctgtcatctgccaggccattaagttattcatggaagatctttgaggaacactgcaagttcatatcataaacacatttgaaattgagtattgttttgcattgtatggagctatgttttgctgtatcctcagaaaaaaagtttgttataaagcattcacacccataaaaagatagatttaaatattccagctataggaaagaaagtgcgtctgctcttcactctagtctcagttggctccttcacatgcatgcttctttatttctcctattttgtcaagaaaataataggtcacgtcttgttctcacttatgtcctgcctagcatggctcagatgcacgttgtagatacaagaaggatcaaatgaaacagacttctggtctgttactacaaccatagtaataagcacactaactaataattgctaattatgttttccatctctaaggttcccacatttttctgttttcttaaagatcccattatctggttgtaactgaagctcaatggaacatgagcaatatttcccagtcttctctcccatccaacagtcctgatggattagcagaacaggcagaaaacacattgttacccagaattaaaaactaatatttgctctccattcaatccaaaatggacctattgaaactaaaatctaacccaatcccattaaatgatttctatggcgtcaaaggtcaaacttctgaagggaacctgtgggtgggtcacaattcaggctatatattccccagggctcag

SEQ ID NO: 71 - DNA, 680 bp, GALLUS SP.
ccgggctgcagaaaaatgccaggtggactatgaactcacatccaaaggagcttgacctgatacctgattttcttcaaactggggaaacaacacaatcccacaaaacagctcagagagaaaccatcactgatggctacagcaccaaggtatgcaatggcaatccattcgacattcatctgtgacctgagcaaaatgatttatctctccatgaatggttgcttctttccctcatgaaaaggcaatttccacactcacaatatgcaacaaagacaaacagagaacaattaatgtgctccttcctaatgtcaaaattgtagtggcaaagaggagaacaaaatctcaagttctgagtaggttttagtgattggataagaggctttgacctgtgagctcacctggacttcatatccttttggataaaaagtgcttttataactttcaggtctccgagtctttattcatgagactgttggtttagggacagacccacaatgaaatgcctggcataggaaagggcagcagagccttagctgaccttttcttgggacaagcattgtcaaacaatgtgtgacaaaactatttgtactgctttgcacagctgtgctgggcagggcaatccattgccacctatcccaggtaaccttccaactgcaagaagattgttgcttactctctctaga

SEQ ID NO: 72 - DNA, 72 bp, ARTIFICIAL SEQUENCE
  Synthetic
gtggatcaacatacagctagaaagctgtattgcctttagcactcaagctcaaaagacaactcagagttcacc

SEQ ID NO: 73 - DNA, 62 bp, ARTIFICIAL SEQUENCE
  Synthetic
acatacagctagaaagctgtattgcctttagcactcaagctcaaaagacaactcagagttca

SEQ ID NO: 74 - DNA, 1158 bp, Gallus
atgggctccatcggcgcagcaagcatggaattttgttttgatgtattcaaggagctcaaagtccaccatgccaatgagaacatcttctactgccccattgccatcatgtcagctctagccatggtatacctgggtgcaaaagacagcaccaggacacagataaataaggttgttcgctttgataaacttccaggattcggagacagtattgaagctcagtgtggcacatctgtaaacgttcactcttcacttagagacatcctcaaccaaatcaccaaaccaaatgatgtttattcgttcagccttgccagtagactttatgctgaagagagatacccaatcctgccagaatacttgcagtgtgtgaaggaactgtatagaggaggcttggaacctatcaactttcaaacagctgcagatcaagccagagagctcatcaattcctgggtagaaagtcagacaaatggaattatcagaaatgtccttcagccaagctccgtggattctcaaactgcaatggttctggttaatgccattgtcttcaaaggactgtgggagaaaacatttaaggatgaagacacacaagcaatgcctttcagagtgactgagcaagaaagcaaacctgtgcagatgatgtaccagattggtttatttagagtggcatcaatggcttctgagaaaatgaagatcctggagcttccatttgccagtgggacaatgagcatgttggtgctgttgcctgatgaagtctcaggccttgagcagcttgagagtataatcaactttgaaaaactgactgaatggaccagttctaatgttatggaagagaggaagatcaaagtgtacttacctcgcatgaagatggaggaaaaatacaacctcacatctgtcttaatggctatgggcattactgacgtgtttagctcttcagccaatctgtctggcatctcctcagcagagagcctgaagatatctcaagctgtccatgcagcacatgcagaaatcaatgaagcaggcagagaggtggtagggtcagcagaggctggagtggatgctgcaagcgtctctgaagaatttagggctgaccatccattcctcttctgtatcaagcacatcgcaaccaacgccgttctcttctttggcagatgtgtttcccct

SEQ ID NO: 75 - DNA, 51 bp, Gallus
atgggctccatcggcgcagcaagcatggaattttgttttgatgtattcaag

SEQ ID NO: 76 - DNA, 105 bp, Gallus
atgggctccatcggcgcagcaagcatggaattttgttttgatgtattcaaggagctcaaagtccaccatgccaatgagaacatcttctactgccccattgccatc

SEQ ID NO: 77 - DNA, 63 bp, ARTIFICIAL SEQUENCE
  Synthetic
atgagggggatcatactggcattagtgctcacccttgtaggcagccagaagtttgacattggt

SEQ ID NO: 78 - protein, 13 aa, ARTIFICIAL SEQUENCE
  Synthetic
KYKKALKKLAKLL

SEQ ID NO: 79 - DNA, 39 bp, ARTIFICIAL SEQUENCE
  Synthetic
aaatacaaaaaagcactgaaaaaactggcaaaactgctg

SEQ ID NO: 80 - DNA, 260 bp, ARTIFICIAL SEQUENCE
  Synthetic
tttgtgaaccaacacctgtgcggctcacacctggtggaagctctctacctagtgtgcggggaacgaggcttcttctacacacccaagacccgccgggaggcagaggacctgcaggtggggcaggtggagctgggcgggggccctggtgcaggcagcctgcagcccttggccctggaggggtccctgcagaagcgtggcattgtggaacaatgctgtaccagcatctgctccctctaccagctggagaactctgcaactag

SEQ ID NO: 81 - protein, 4 aa, ARTIFICIAL SEQUENCE
  Synthetic
GPGG

SEQ ID NO: 82 - protein, 12 aa, ARTIFICIAL SEQUENCE
  Synthetic
GPGGGPGGGPGG

SEQ ID NO: 83 - protein, 15 aa, ARTIFICIAL SEQUENCE
  Synthetic
GGGGSGGGGSGGGGS

SEQ ID NO: 84 - protein, 20 aa, ARTIFICIAL SEQUENCE
  Synthetic
GGGGSGGGGSGGGGSGGGGS

SEQ ID NO: 85 - protein, 5 aa, ARTIFICIAL SEQUENCE
  Synthetic
PADDA

SEQ ID NO: 86 - protein, 29 aa, ARTIFICIAL SEQUENCE
  Synthetic
PADDAPADDAPADDAPADDAPADDAPADD

SEQ ID NO: 87 - protein, 16 aa, ARTIFICIAL SEQUENCE
  Synthetic
ATTCILKGSCGWIGLL

SEQ ID NO: 88 - protein, 30 aa, ARTIFICIAL SEQUENCE
  Synthetic
PADDAPADDATTCILKGSCGWIGLLDDDDK

SEQ ID NO: 89 - protein, 5 aa, ARTIFICIAL SEQUENCE
  Synthetic
DDDDK

SEQ ID NO: 90 - protein, 50 aa, ARTIFICIAL SEQUENCE
  Synthetic
PADDAPADDAPADDAPADDAPADDAPADDATTCILKGSCGWIGLLDDDDK

SEQ ID NO: 91 - DNA, 48 bp, ARTIFICIAL SEQUENCE
  Synthetic
atctcgagaccatgtgtgaacttgatattttacatgattctctttacc

SEQ ID NO: 92 - DNA, 36 bp, ARTIFICIAL SEQUENCE
  Synthetic
gattgatcattatcataatttccccaaagcgtaacc

SEQ ID NO: 93 - DNA, 6 bp, ARTIFICIAL SEQUENCE
  Synthetic
ctcgag

SEQ ID NO: 94 - DNA, 6 bp, ARTIFICIAL SEQUENCE
  Synthetic
tgatca

SEQ ID NO: 95 - DNA, 22 bp, ARTIFICIAL SEQUENCE
  Synthetic
ttgccggcatcagattggctat

SEQ ID NO: 96 - DNA, 34 bp, ARTIFICIAL SEQUENCE
  Synthetic
agaggtcaccgggtcaattcttcagcacctggta

SEQ ID NO: 97 - DNA, 11973 bp, ARTIFICIAL SEQUENCE
  Synthetic
ctgacgcgccctgtagcggcgcattaagcgcggcgggtgtggtggttacgcgcagcgtgaccgctacacttgccagcgccctagcgcccgctcctttcgctttcttcccttcctttctcgccacgttcgccggcatcagattggctattggccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccgcggccgggaacggtgcattggaacgcggattccccgtgccaagagtgacgtaagtaccgcctatagactctataggcacacccctttggctcttatgcatgctatactgtttttggcttggggcctatacacccccgcttccttatgctataggtgatggtatagcttagcctataggtgtgggttattgaccattattgaccactcccctattggtgacgatactttccattactaatccataacatggctctttgccacaactatctctattggctatatgccaatactctgtccttcagagactgacacggactctgtatttttacaggatggggtcccatttattatttacaaattcacatatacaacaacgccgtcccccgtgcccgcagtttttattaaacatagcgtgggatctccacgcgaatctcgggtacgtgttccggacatgggctcttctccggtagcggcggagcttccacatccgagccctggtcccatgcctccagcggctcatggtcgctcggcagctccttgctcctaacagtggaggccagacttaggcacagcacaatgcccaccaccaccagtgtgccgcacaaggccgtggcggtagggtatgtgtctgaaaatgagcgtggagattgggctcgcacggctgacgcagatggaagacttaaggcagcggcagaagaagatgcaggcagctgagttgttgtattctgataagagtcagaggtaactcccgttgcggtgctgttaacggtggagggcagtgtagtctgagcagtactcgttgctgccgcgcgcgccaccagacataatagctgacagactaacagactgttcctttccatgggtcttttctgcagtcaccgtcggaccatgtgtgaacttgatattttacatgattctctttaccaattctgccccgaattacacttaaaacgactcaacagcttaacgttggcttgccacgcattacttgactgtaaaactctcactcttaccgaacttggccgtaacctgccaaccaaagcgagaacaaaacataacatcaaacgaatcgaccgattgttaggtaatcgtcacctccacaaagagcgactcgctgtataccgttggcatgctagctttatctgttcgggcaatacgatgcccattgtacttgttgactggtctgatattcgtgagcaaaaacgacttatggtattgcgagcttcagtcgcactacacggtcgttctgttactctttatgagaaagcgttcccgctttcagagcaatgttcaaagaaagctcatgaccaatttctagccgaccttgcgagcattctaccgagtaacaccacaccgctcattgtcagtgatgctggctttaaagtgccatggtataaatccgttgagaagctgggttggtactggttaagtcgagtaagaggaaaagtacaatatgcagacctaggagcggaaaactggaaacctatcagcaacttacatgatatgtcatctagtcactcaaagactttaggctataagaggctgactaaaagcaatccaatctcatgccaaattctattgtataaatctcgctctaaaggccgaaaaaatcagcgctcgacacggactcattgtcaccacccgtcacctaaaatctactcagcgtcggcaaaggagccatgggttctagcaactaacttacctgttgaaattcgaacacccaaacaacttgttaatatctattcgaagcgaatgcagattgaagaaaccttccgagacttgaaaagtcctgcctacggactaggcctacgccatagccgaacgagcagctcagagcgttttgatatcatgctgctaatcgccctgatgcttcaactaacatgttggcttgcgggcgttcatgctcagaaacaaggttgggacaagcacttccaggctaacacagtcagaaatcgaaacgtactctcaacagttcgcttaggcatggaagttttgcggcattctggctacacaataacaagggaagacttactcgtggctgcaaccctactagctcaaaatttattcacacatggttacgctttggggaaattatgataatgatccagatcacttctggctaataaaagatcagagctctagagatctgtgtgttggttttttgtggatctgctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcagcacagcaagggggaggattgggaagacaatagcaggcatgctggggatgcggtgggctctatgggtacctctctctctctctctctctctctctctctctctctctctcggtacctctctctctctctctctctctctctctctctctctctctcggtaccaggtgctgaagaattgacccggtgaccaaaggtgccttttatcatcactttaaaaataaaaaacaattactcagtgcctgttataagcagcaattaattatgattgatgcctacatcacaacaaaaactgatttaacaaatggttggtctgccttagaaagtatatttgaacattatcttgattatattattgataataataaaaaccttatccctatccaagaagtgatgcctatcattggttggaatgaacttgaaaaaaattagccttgaatacattactggtaaggtaaacgccattgtcagcaaattgatccaagagaaccaacttaaagctttcctgacggaatgttaattctcgttgaccctgagcactgatgaatcccctaatgattttggtaaaaatcattaagttaaggtggatacacatcttgtcatatgatcccggtaatgtgagttagctcactcattaggcaccccaggctttacactttatgcttccggctcgtatgttgtgtggaattgtgagcggataacaatttcacacaggaaacagctatgaccatgattacgccaagcgcgcaattaaccctcactaaagggaacaaaagctggagctccaccgcggtggcggccgctctagaactagtggatcccccgggctgcaggaattcgatatcaagcttatcgataccgctgacctcgagcatcagattggctattggccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgtcaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtcttcaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccgcggccgggaacggtgcattggaacgcggattccccgtgccaagagtgacgtaagtaccgcctatagactctataggcacacccctttggctcttatgcatgctatactgtttttggcttggggcctatacacccccgcttccttatgctataggtgatggtatagcttagcctataggtgtgggttattgaccattattgaccactcccctattggtgacgatactttccattactaatccataacatggctctttgccacaactatctctattggctatatgccaatactctgtccttcagagactgacacggactctgtatttttacaggatggggtcccatttattatttacaaattcacatatacaacaacgccgtcccccgtgcccgcagtttttattaaacatagcgtgggatctccacgcgaatctcgggtacgtgttccggacatgggctcttctccggtagcggcggagcttccacatccgagccctggtcccatgcctccagcggctcatggtcgctcggcagctccttgctcctaacagtggaggccagacttaggcacagcacaatgcccaccaccaccagtgtgccgcacaaggccgtggcggtagggtatgtgtctgaaaatgagcgtggagattgggctcgcacggctgacgcagatggaagacttaaggcagcggcagaagaagatgcaggcagctgagttgttgtattctgataagagtcagaggtaactcccgttgcggtgctgttaacggtggagggcagtgtagtctgagcagtactcgttgctgccgcgcgcgccaccagacataatagctgacagactaacagactgttcctttccatgggtcttttctgcagtcaccgtcggatcaatcattcatctcgtgacttcttcgtgtgtggtgtttacctatatatctaaatttaatatttcgtttattaaaatttaatatatttcgacgatgaatttctcaaggatatttttcttcgtgttcgctttggttctggctttgtcaacagtttcggctgcgccagagccgaaaggtacccaggtgcagctgcaggagtcggggggaggcttggtaaagccgggggggtcccttagagtctcctgtgcagcctctggattcactttcagaaacgcctggatgagctgggtccgccaggctccagggaaggggctggagtgggtcggccgtattaaaagcaaaattgatggtgggacaacagactatgctgcacccgtgaaaggcagattcaccatctcaagagatgattcaaaaaacacgttatatctgcaaatgaatagcctgaaagccgaggacacagccgtatattactgtaccacggggattatgataacatttgggggagttatccctcccccgaattggggccagggaaccctggtcaccgtctcctcagcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacacctttccggctgtcctacagtcctcaggactctacttccttagcaacgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaagcgccagagccgaaaaagctttcctatgagctgacacagccaccctcggtgtcagtgtccccaggacaaacggccaggatcacctgctctggagatgcattgccagaaaaatatgtttattggtaccagcagaagtcaggccaggcccctgtggtggtcatctatgaggacagcaaacgaccctccgggatccctgagagattctctggctccagctcagggacaatggccaccttgactatcagtggggcccaggtggaagatgaaggtgactactactgttactcaactgacagcagtggttatcatagggaggtgttcagcggagggaccaagctgaccgtcctaggtcagcccaaggctgccccctcggtcactctgttcccaccctcctctgaggagcttcaagccaacaaggccacactggtgtgtctcataagtgactcctacccgggagccgtgacagtggcctggaaggcagatagcagccccgtcaaggcgggagtggagaccaccacaccctccaaacaaagcaacaacaagtacgcggccagcagctacctgagcctgacgcttgagcagtggaagtcccacaaaagctacagctgccaggtcacgcatgaagggagcaccgtggagaagacagtggcccctgcagaatgttcaccgcggagggagggaagggccctttttgaagggggaggaaacttcgcgccatgactcctctcgtgccccccgcacggaacactgatgtgcagagggccctctgccattgctgcttcctctgcccttcctcgtcactctgaatgtggcttctttgctactgccacagcaagaaataaaatctcaacatctaaatgggtttcctgagatttttcaagagtcgttaagcacattccttccccagcaccccttgctgcaggccagtgccaggcaccaacttggctactgctgcccatgagagaaatccagttcaatattttccaaagcaaaatggattacatatgccctagatcctgattaacaggtgttttgtattatctgtgctttcgcttcacccacattatcccattgcctcccctcgactcgagggggggcccggtacccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactactccgggatcatatgacaagatgtgtatccaccttaacttaatgatttttaccaaaatcattaggggattcatcagtgctcagggtcaacgagaattaacattccgtcaggaaagcttatgatgatgatgtgcttaaaaacttactcaatggctggttatgcatatcgcaatacatgcgaaaaacctaaaagagcttgccgataaaaaaggccaatttattgctatttaccgcggctttttattgagcttgaaagataaataaaatagataggttttatttgaagctaaatcttctttatcgtaaaaaatgccctcttgggttatcaagagggtcattatatttcgcggaataacatcatttggtgacgaaataactaagcacttgtctcctgtttactcccctgagcttgaggggttaacatgaaggtcatcgatagcaggataataatacagtaaaacgctaaaccaataatccaaatccagccatcccaaattggtagtgaatgattataaataacagcaaacagtaatgggccaataacaccggttgcattggtaaggctcaccaataatccctgtaaagcaccttgctgatgactctttgtttggatagacatcactccctgtaatgcaggtaaagcgatcccaccaccagccaataaaattaaaacagggaaaactaaccaaccttcagatataaacgctaaaaaggcaaatgcactactatctgcaataaatccgagcagtactgccgttttttcgcccatttagtggctattcttcctgccacaaaggcttggaatactgagtgtaaaagaccaagacccgtaatgaaaagccaaccatcatgctattcatcatcacgatttctgtaatagcaccacaccgtgctggattggctatcaatgcgctgaaataataatcaacaaatggcatcgttaaataagtgatgtataccgatcagcttttgttccctttagtgagggttaattgcgcgcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccac

SEQ ID NO: 98 - DNA, 11964 bp, ARTIFICIAL SEQUENCE
  Synthetic
ctgacgcgccctgtagcggcgcattaagcgcggcgggtgtggtggttacgcgcagcgtgaccgctacacttgccagcgccctagcgcccgctcctttcgctttcttcccttcctttctcgccacgttcgccggcatcagattggctattggccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccgcggccgggaacggtgcattggaacgcggattccccgtgccaagagtgacgtaagtaccgcctatagactctataggcacacccctttggctcttatgcatgctatactgtttttggcttggggcctatacacccccgcttccttatgctataggtgatggtatagcttagcctataggtgtgggttattgaccattattgaccactcccctattggtgacgatactttccattactaatccataacatggctctttgccacaactatctctattggctatatgccaatactctgtccttcagagactgacacggactctgtatttttacaggatggggtcccatttattatttacaaattcacatatacaacaacgccgtcccccgtgcccgcagtttttattaaacatagcgtgggatctccacgcgaatctcgggtacgtgttccggacatgggctcttctccggtagcggcggagcttccacatccgagccctggtcccatgcctccagcggctcatggtcgctcggcagctccttgctcctaacagtggaggccagacttaggcacagcacaatgcccaccaccaccagtgtgccgcacaaggccgtggcggtagggtatgtgtctgaaaatgagcgtggagattgggctcgcacggctgacgcagatggaagacttaaggcagcggcagaagaagatgcaggcagctgagttgttgtattctgataagagtcagaggtaactcccgttgcggtgctgttaacggtggagggcagtgtagtctgagcagtactcgttgctgccgcgcgcgccaccagacataatagctgacagactaacagactgttcctttccatgggtcttttctgcagtcaccgtcggaccatgtgcgaactcgatattttacacgactctctttaccaattctgccccgaattacacttaaaacgactcaacagcttaacgttggcttgccacgcattacttgactgtaaaactctcactcttaccgaacttggccgtaacctgccaaccaaagcgagaacaaaacataacatcaaacgaatcgaccgattgttaggtaatcgtcacctccacaaagagcgactcgctgtataccgttggcatgctagctttatctgttcgggcaatacgatgcccattgtacttgttgactggtctgatattcgtgagcaaaaacgacttatggtattgcgagcttcagtcgcactacacggtcgttctgttactctttatgagaaagcgttcccgctttcagagcaatgttcaaagaaagctcatgaccaatttctagccgaccttgcgagcattctaccgagtaacaccacaccgctcattgtcagtgatgctggctttaaagtgccatggtataaatccgttgagaagctgggttggtactggttaagtcgagtaagaggaaaagtacaatatgcagacctaggagcggaaaactggaaacctatcagcaacttacatgatatgtcatctagtcactcaaagactttaggctataagaggctgactaaaagcaatccaatctcatgccaaattctattgtataaatctcgctctaaaggccgaaaaaatcagcgctcgacacggactcattgtcaccacccgtcacctaaaatctactcagcgtcggcaaaggagccatgggttctagcaactaacttacctgttgaaattcgaacacccaaacaacttgttaatatctattcgaagcgaatgcagattgaagaaaccttccgagacttgaaaagtcctgcctacggactaggcctacgccatagccgaacgagcagctcagagcgttttgatatcatgctgctaatcgccctgatgcttcaactaacatgttggcttgcgggcgttcatgctcagaaacaaggttgggacaagcacttccaggctaacacagtcagaaatcgaaacgtactctcaacagttcgcttaggcatggaagttttgcggcattctggctacacaataacaagggaagacttactcgtggctgcaaccctactagctcaaaatttattcacacatggttacgctttggggaaattatgaggggatcgctctagagcgatccgggatctcgggaaaagcgttggtgaccaaaggtgccttttatcatcactttaaaaataaaaaacaattactcagtgcctgttataagcagcaattaattatgattgatgcctacatcacaacaaaaactgatttaacaaatggttggtctgccttagaaagtatatttgaacattatcttgattatattattgataataataaaaaccttatccctatccaagaagtgatgcctatcattggttggaatgaacttgaaaaaaattagccttgaatacattactggtaaggtaaacgccattgtcagcaaattgatccaagagaaccaacttaaagctttcctgacggaatgttaattctcgttgaccctgagcactgatgaatcccctaatgattttggtaaaaatcattaagttaaggtggatacacatcttgtcatatgatcccggtaatgtgagttagctcactcattaggcaccccaggctttacactttatgcttccggctcgtatgttgtgtggaattgtgagcggataacaatttcacacaggaaacagctatgaccatgattacgccaagcgcgcaattaaccctcactaaagggaacaaaagctggagctccaccgcggtggcggccgctctagaactagtggatcccccgggctgcaggaattcgatatcaagcttatcgataccgctgacctcgagctgcagaaaaatgccaggtggactatgaactcacatccaaaggagcttgacctgatacctgattttcttcaaacaggggaaacaacacaatcccacaaaacagctcagagagaaaccatcactgatggctacagcaccaaggtatgcaatggcaatccattcgacattcatctgtgacctgagcaaaatgatttctctctccatgaatggttgcttctttccctcatgaaaaggcaatttccacactcacaatatgcgacaaagacaaacagagaacaattaatgtgctccttcctaatgtcaaaattgtagtggcaaagaggagaacaaaatctcaagttctgagtaggttttagtgattggataagaggctttgacctgtgagctcacctggacttcatatccttttggataaaaagtgcttttataactttcaggtctccgagtctttattcatgagactgttggtttagggacagacccacaatgaaatgcctggcataggaaagggcagcagagccttagctgaccttttcttgggacaagcattgtcaaacaatgtgtgacaaaactatttgtactgctttgcacagctgtgctgggcagggcgatccattgccacctatcccaggtaaccttccaactgcaagaagattgttgcttactctctctagaaagcttctgcagactgacatgcatttcataggtagagataacatttactgggaagcacatctatcatcacaaaaagcaggcaagattttcagactttcttagtggctgaaatagaagcaaaagacgtaattaaaaacaaaatgaaacaaaaaaaatcagttgatacctgtggtgtagacatccagcaaaaaaatattatttgcactaccatcttgtcttaagtcctcagacttagcaaggagaatgtagatttccacagtatatatgttttcacaaaaggaaggagagaaacaaaagaaaatggcactgactaaacttcagctagtggtataggaaagtaattctgcttaacagagattgcagtgatctctatgtatgtcctgaagaattatgttgtacttttttcccccatttttaaatcaaacagtgctttacagaggtcagaatggtttctttactgtttgtcaattctattatttcaatacagaacaatagcttctataactgaaatatatttgctattgtatattatgattgtccctcgaaccatgaacactcctccagctgaatttcacaattcctctgtcatctgccaggccattaagttattcatggaagatctttgaggaacactgcaagttcatatcataaacacatttgaaattgagtattggtttgcattgtatggagctatgttttgctgtatcctcagaaaaaaagtttgttataaagcattcacacccataaaaagatagatttaaatattccagctataggaaagaaagtgcgtctgctcttcactctagtctcagttggctccttcacatgcatgcttctttatttctcctattttgtcaagaaaataataggtcacgtcttgttctcacttatgtcctgcctagcatggctcagatgcacgttgtagatacaagaaggatcaaatgaaacagacttctggtctgttacctacaaccatagtaataagcacactaactaataattgctaattatgttttccatctctaaggttcccatatttttctgttttcttaaagatcccattatctggttgtaactgaagctcaatggaacatgagcaatatttcccagtcttctctcccatccaacagtcctgatggattagcagaacaggcagaaaacacattgttacccagaattaaaaactaatatttgctctccattcaatccaaaatggacctattgaaactaaaatctaacccaatcccattaaatgatttctatggtgtcaaaggtcaaacttctgaagggaacctgtgggtgggtcacaattcaggctatatattccccagggctcagccagtggatcaatcattcatctcgtgacttcttcgtgtgtggtgtttacctatatatctaaatttaatatttcgtttattaaaatttaatatatttcgacgatgaatttctcaaggatatttttcttcgtgttcgctttggttctggctttgtcaacagtttcggctgcgccagagccgaaaggtacccaggtgcagctgcaggagtcggggggaggcttggtaaagccgggggggtcccttagagtctcctgtgcagcctctggattcactttcagaaacgcctggatgagctgggtccgccaggctccagggaaggggctggagtgggtcggccgtattaaaagcaaaattgatggtgggacaacagactatgctgcacccgtgaaaggcagattcaccatctcaagagatgattcaaaaaacacgttatatctgcaaatgaatagcctgaaagccgaggacacagccgtatattactgtaccacggggattatgataacatttgggggagttatccctcccccgaattggggccagggaaccctggtcaccgtctcctcagcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacacctttccggctgtcctacagtcctcaggactctacttccttagcaacgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaagcgccagagccgaagctttcctatgagctgacacagccaccctcggtgtcagtgtccccaggacaaacggccaggatcacctgctctggagatgcattgccagaaaaatatgtttattggtaccagcagaagtcaggccaggcccctgtggtggtcatctatgaggacagcaaacgaccctccgggatccctgagagattctctggctccagctcagggacaatggccaccttgactatcagtggggcccaggtggaagatgaaggtgactactactgttactcaactgacagcagtggttatcatagggaggtgttcagcggagggaccaagctgaccgtcctaggtcagcccaaggctgccccctcggtcactctgttcccaccctcctctgaggagcttcaagccaacaaggccacactggtgtgtctcataagtgactcctacccgggagccgtgacagtggcctggaaggcagatagcagccccgtcaaggcgggagtggagaccaccacaccctccaaacaaagcaacaacaagtacgcggccagcagctacctgagcctgacgcttgagcagtggaagtcccacaaaagctacagctgccaggtcacgcatgaagggagcaccgtggagaagacagtggcccctgcagaatgttcaccgcggagggagggaagggccctttttgaagggggaggaaacttcgcgccatgactcctctcgtgccccccgcacggaacactgatgtgcagagggccctctgccattgctgcttcctctgcccttcctcgtcactctgaatgtggcttctttgctactgccacagcaagaaataaaatctcaacatctaaatgggtttcctgagatttttcaagagtcgttaagcacattccttccccagcaccccttgctgcaggccagtgccaggcaccaacttggctactgctgcccatgagagaaatccagttcaatattttccaaagcaaaatggattacatatgccctagatcctgattaacaggtgttttgtattatctgtgctttcgcttcacccacattatcccattgcctcccctcgagggggggcccggtacccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactactccgggatcatatgacaagatgtgtatccaccttaacttaatgatttttaccaaaatcattaggggattcatcagtgctcagggtcaacgagaattaacattccgtcaggaaagcttatgatgatgatgtgcttaaaaacttactcaatggctggttatgcatatcgcaatacatgcgaaaaacctaaaagagcttgccgataaaaaaggccaatttattgctatttaccgcggctttttattgagcttgaaagataaataaaatagataggttttatttgaagctaaatcttctttatcgtaaaaaatgccctcttgggttatcaagagggtcattatatttcgcggaataacatcatttggtgacgaaataactaagcacttgtctcctgtttactcccctgagcttgaggggttaacatgaaggtcatcgatagcaggataataatacagtaaaacgctaaaccaataatccaaatccagccatcccaaattggtagtgaatgattataaataacagcaaacagtaatgggccaataacaccggttgcattggtaaggctcaccaataatccctgtaaagcaccttgctgatgactctttgtttggatagacatcactccctgtaatgcaggtaaagcgatcccaccaccagccaataaaattaaaacagggaaaactaaccaaccttcagatataaacgctaaaaaggcaaatgcactactatctgcaataaatccgagcagtactgccgttttttcgcccatttagtggctattcttcctgccacaaaggcttggaatactgagtgtaaaagaccaagacccgtaatgaaaagccaaccatcatgctattcatcatcacgatttctgtaatagcaccacaccgtgctggattggctatcaatgcgctgaaataataatcaacaaatggcatcgttaaataagtgatgtataccgatcagcttttgttccctttagtgagggttaattgcgcgcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccac

SEQ ID NO: 99 - DNA, 11967 bp, ARTIFICIAL SEQUENCE
  Synthetic
ctgacgcgccctgtagcggcgcattaagcgcggcgggtgtggtggttacgcgcagcgtgaccgctacacttgccagcgccctagcgcccgctcctttcgctttcttcccttcctttctcgccacgttcgccggcatcagattggctattggccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccgcggccgggaacggtgcattggaacgcggattccccgtgccaagagtgacgtaagtaccgcctatagactctataggcacacccctttggctcttatgcatgctatactgtttttggcttggggcctatacacccccgcttccttatgctataggtgatggtatagcttagcctataggtgtgggttattgaccattattgaccactcccctattggtgacgatactttccattactaatccataacatggctctttgccacaactatctctattggctatatgccaatactctgtccttcagagactgacacggactctgtatttttacaggatggggtcccatttattatttacaaattcacatatacaacaacgccgtcccccgtgcccgcagtttttattaaacatagcgtgggatctccacgcgaatctcgggtacgtgttccggacatgggctcttctccggtagcggcggagcttccacatccgagccctggtcccatgcctccagcggctcatggtcgctcggcagctccttgctcctaacagtggaggccagacttaggcacagcacaatgcccaccaccaccagtgtgccgcacaaggccgtggcggtagggtatgtgtctgaaaatgagcgtggagattgggctcgcacggctgacgcagatggaagacttaaggcagcggcagaagaagatgcaggcagctgagttgttgtattctgataagagtcagaggtaactcccgttgcggtgctgttaacggtggagggcagtgtagtctgagcagtactcgttgctgccgcgcgcgccaccagacataatagctgacagactaacagactgttcctttccatgggtcttttctgcagtcaccgtcggaccatgtgcgaactcgatattttacacgactctctttaccaattctgccccgaattacacttaaaacgactcaacagcttaacgttggcttgccacgcattacttgactgtaaaactctcactcttaccgaacttggccgtaacctgccaaccaaagcgagaacaaaacataacatcaaacgaatcgaccgattgttaggtaatcgtcacctccacaaagagcgactcgctgtataccgttggcatgctagctttatctgttcgggcaatacgatgcccattgtacttgttgactggtctgatattcgtgagcaaaaacgacttatggtattgcgagcttcagtcgcactacacggtcgttctgttactctttatgagaaagcgttcccgctttcagagcaatgttcaaagaaagctcatgaccaatttctagccgaccttgcgagcattctaccgagtaacaccacaccgctcattgtcagtgatgctggctttaaagtgccatggtataaatccgttgagaagctgggttggtactggttaagtcgagtaagaggaaaagtacaatatgcagacctaggagcggaaaactggaaacctatcagcaacttacatgatatgtcatctagtcactcaaagactttaggctataagaggctgactaaaagcaatccaatctcatgccaaattctattgtataaatctcgctctaaaggccgaaaaaatcagcgctcgacacggactcattgtcaccacccgtcacctaaaatctactcagcgtcggcaaaggagccatgggttctagcaactaacttacctgttgaaattcgaacacccaaacaacttgttaatatctattcgaagcgaatgcagattgaagaaaccttccgagacttgaaaagtcctgcctacggactaggcctacgccatagccgaacgagcagctcagagcgttttgatatcatgctgctaatcgccctgatgcttcaactaacatgttggcttgcgggcgttcatgctcagaaacaaggttgggacaagcacttccaggctaacacagtcagaaatcgaaacgtactctcaacagttcgcttaggcatggaagttttgcggcattctggctacacaataacaagggaagacttactcgtggctgcaaccctactagctcaaaatttattcacacatggttacgctttggggaaattatgaggggatcgctctagagcgatccgggatctcgggaaaagcgttggtgaccaaaggtgccttttatcatcactttaaaaataaaaaacaattactcagtgcctgttataagcagcaattaattatgattgatgcctacatcacaacaaaaactgatttaacaaatggttggtctgccttagaaagtatatttgaacattatcttgattatattattgataataataaaaaccttatccctatccaagaagtgatgcctatcattggttggaatgaacttgaaaaaaattagccttgaatacattactggtaaggtaaacgccattgtcagcaaattgatccaagagaaccaacttaaagctttcctgacggaatgttaattctcgttgaccctgagcactgatgaatcccctaatgattttggtaaaaatcattaagttaaggtggatacacatcttgtcatatgatcccggtaatgtgagttagctcactcattaggcaccccaggctttacactttatgcttccggctcgtatgttgtgtggaattgtgagcggataacaatttcacacaggaaacagctatgaccatgattacgccaagcgcgcaattaaccctcactaaagggaacaaaagctggagctccaccgcggtggcggccgctctagaactagtggatcccccgggctgcaggaattcgatatcaagcttatcgataccgctgacctcgagctgcagaaaaatgccaggtggactatgaactcacatccaaaggagcttgacctgatacctgattttcttcaaacaggggaaacaacacaatcccacaaaacagctcagagagaaaccatcactgatggctacagcaccaaggtatgcaatggcaatccattcgacattcatctgtgacctgagcaaaatgatttctctctccatgaatggttgcttctttccctcatgaaaaggcaatttccacactcacaatatgcgacaaagacaaacagagaacaattaatgtgctccttcctaatgtcaaaattgtagtggcaaagaggagaacaaaatctcaagttctgagtaggttttagtgattggataagaggctttgacctgtgagctcacctggacttcatatccttttggataaaaagtgcttttataactttcaggtctccgagtctttattcatgagactgttggtttagggacagacccacaatgaaatgcctggcataggaaagggcagcagagccttagctgaccttttcttgggacaagcattgtcaaacaatgtgtgacaaaactatttgtactgctttgcacagctgtgctgggcagggcgatccattgccacctatcccaggtaaccttccaactgcaagaagattgttgcttactctctctagaaagcttctgcagactgacatgcatttcataggtagagataacatttactgggaagcacatctatcatcacaaaaagcaggcaagattttcagactttcttagtggctgaaatagaagcaaaagacgtaattaaaaacaaaatgaaacaaaaaaaatcagttgatacctgtggtgtagacatccagcaaaaaaatattatttgcactaccatcttgtcttaagtcctcagacttagcaaggagaatgtagatttccacagtatatatgttttcacaaaaggaaggagagaaacaaaagaaaatggcactgactaaacttcagctagtggtataggaaagtaattctgcttaacagagattgcagtgatctctatgtatgtcctgaagaattatgttgtacttttttcccccatttttaaatcaaacagtgctttacagaggtcagaatggtttctttactgtttgtcaattctattatttcaatacagaacaatagcttctataactgaaatatatttgctattgtatattatgattgtccctcgaaccatgaacactcctccagctgaatttcacaattcctctgtcatctgccaggccattaagttattcatggaagatctttgaggaacactgcaagttcatatcataaacacatttgaaattgagtattggtttgcattgtatggagctatgttttgctgtatcctcagaaaaaaagtttgttataaagcattcacacccataaaaagatagatttaaatattccagctataggaaagaaagtgcgtctgctcttcactctagtctcagttggctccttcacatgcatgcttctttatttctcctattttgtcaagaaaataataggtcacgtcttgttctcacttatgtcctgcctagcatggctcagatgcacgttgtagatacaagaaggatcaaatgaaacagacttctggtctgttacctacaaccatagtaataagcacactaactaataattgctaattatgttttccatctctaaggttcccatatttttctgttttcttaaagatcccattatctggttgtaactgaagctcaatggaacatgagcaatatttcccagtcttctctcccatccaacagtcctgatggattagcagaacaggcagaaaacacattgttacccagaattaaaaactaatatttgctctccattcaatccaaaatggacctattgaaactaaaatctaacccaatcccattaaatgatttctatggtgtcaaaggtcaaacttctgaagggaacctgtgggtgggtcacaattcaggctatatattccccagggctcagccagtggatcaatcattcatctcgtgacttcttcgtgtgtggtgtttacctatatatctaaatttaatatttcgtttattaaaatttaatatatttcgacgatgaatttctcaaggatatttttcttcgtgttcgctttggttctggctttgtcaacagtttcggctgcgccagagccgaaaggtacccaggtgcagctgcaggagtcggggggaggcttggtaaagccgggggggtcccttagagtctcctgtgcagcctctggattcactttcagaaacgcctggatgagctgggtccgccaggctccagggaaggggctggagtgggtcggccgtattaaaagcaaaattgatggtgggacaacagactatgctgcacccgtgaaaggcagattcaccatctcaagagatgattcaaaaaacacgttatatctgcaaatgaatagcctgaaagccgaggacacagccgtatattactgtaccacggggattatgataacatttgggggagttatccctcccccgaattggggccagggaaccctggtcaccgtctcctcagcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacacctttccggctgtcctacagtcctcaggactctacttccttagcaacgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaagcgccagagccgaaaaagctttcctatgagctgacacagccaccctcggtgtcagtgtccccaggacaaacggccaggatcacctgctctggagatgcattgccagaaaaatatgtttattggtaccagcagaagtcaggccaggcccctgtggtggtcatctatgaggacagcaaacgaccctccgggatccctgagagattctctggctccagctcagggacaatggccaccttgactatcagtggggcccaggtggaagatgaaggtgactactactgttactcaactgacagcagtggttatcatagggaggtgttcagcggagggaccaagctgaccgtcctaggtcagcccaaggctgccccctcggtcactctgttcccaccctcctctgaggagcttcaagccaacaaggccacactggtgtgtctcataagtgactcctacccgggagccgtgacagtggcctggaaggcagatagcagccccgtcaaggcgggagtggagaccaccacaccctccaaacaaagcaacaacaagtacgcggccagcagctacctgagcctgacgcttgagcagtggaagtcccacaaaagctacagctgccaggtcacgcatgaagggagcaccgtggagaagacagtggcccctgcagaatgttcaccgcggagggagggaagggccctttttgaagggggaggaaacttcgcgccatgactcctctcgtgccccccgcacggaacactgatgtgcagagggccctctgccattgctgcttcctctgcccttcctcgtcactctgaatgtggcttctttgctactgccacagcaagaaataaaatctcaacatctaaatgggtttcctgagatttttcaagagtcgttaagcacattccttccccagcaccccttgctgcaggccagtgccaggcaccaacttggctactgctgcccatgagagaaatccagttcaatattttccaaagcaaaatggattacatatgccctagatcctgattaacaggtgttttgtattatctgtgctttcgcttcacccacattatcccattgcctcccctcgagggggggcccggtacccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactactccgggatcatatgacaagatgtgtatccaccttaacttaatgatttttaccaaaatcattaggggattcatcagtgctcagggtcaacgagaattaacattccgtcaggaaagcttatgatgatgatgtgcttaaaaacttactcaatggctggttatgcatatcgcaatacatgcgaaaaacctaaaagagcttgccgataaaaaaggccaatttattgctatttaccgcggctttttattgagcttgaaagataaataaaatagataggttttatttgaagctaaatcttctttatcgtaaaaaatgccctcttgggttatcaagagggtcattatatttcgcggaataacatcatttggtgacgaaataactaagcacttgtctcctgtttactcccctgagcttgaggggttaacatgaaggtcatcgatagcaggataataatacagtaaaacgctaaaccaataatccaaatccagccatcccaaattggtagtgaatgattataaataacagcaaacagtaatgggccaataacaccggttgcattggtaaggctcaccaataatccctgtaaagcaccttgctgatgactctttgtttggatagacatcactccctgtaatgcaggtaaagcgatcccaccaccagccaataaaattaaaacagggaaaactaaccaaccttcagatataaacgctaaaaaggcaaatgcactactatctgcaataaatccgagcagtactgccgttttttcgcccatttagtggctattcttcctgccacaaaggcttggaatactgagtgtaaaagaccaagacccgtaatgaaaagccaaccatcatgctattcatcatcacgatttctgtaatagcaccacaccgtgctggattggctatcaatgcgctgaaataataatcaacaaatggcatcgttaaataagtgatgtataccgatcagcttttgttccctttagtgagggttaattgcgcgcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccac

SEQ ID NO: 100 - DNA, 11590 bp, ARTIFICIAL SEQUENCE
  Synthetic
ctgacgcgccctgtagcggcgcattaagcgcggcgggtgtggtggttacgcgcagcgtgaccgctacacttgccagcgccctagcgcccgctcctttcgctttcttcccttcctttctcgccacgttcgccggcatcagattggctattggccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccgcggccgggaacggtgcattggaacgcggattccccgtgccaagagtgacgtaagtaccgcctatagactctataggcacacccctttggctcttatgcatgctatactgtttttggcttggggcctatacacccccgcttccttatgctataggtgatggtatagcttagcctataggtgtgggttattgaccattattgaccactcccctattggtgacgatactttccattactaatccataacatggctctttgccacaactatctctattggctatatgccaatactctgtccttcagagactgacacggactctgtatttttacaggatggggtcccatttattatttacaaattcacatatacaacaacgccgtcccccgtgcccgcagtttttattaaacatagcgtgggatctccacgcgaatctcgggtacgtgttccggacatgggctcttctccggtagcggcggagcttccacatccgagccctggtcccatgcctccagcggctcatggtcgctcggcagctccttgctcctaacagtggaggccagacttaggcacagcacaatgcccaccaccaccagtgtgccgcacaaggccgtggcggtagggtatgtgtctgaaaatgagcgtggagattgggctcgcacggctgacgcagatggaagacttaaggcagcggcagaagaagatgcaggcagctgagttgttgtattctgataagagtcagaggtaactcccgttgcggtgctgttaacggtggagggcagtgtagtctgagcagtactcgttgctgccgcgcgcgccaccagacataatagctgacagactaacagactgttcctttccatgggtcttttctgcagtcaccgtcggaccatgtgcgaactcgatattttacacgactctctttaccaattctgccccgaattacacttaaaacgactcaacagcttaacgttggcttgccacgcattacttgactgtaaaactctcactcttaccgaacttggccgtaacctgccaaccaaagcgagaacaaaacataacatcaaacgaatcgaccgattgttaggtaatcgtcacctccacaaagagcgactcgctgtataccgttggcatgctagctttatctgttcgggcaatacgatgcccattgtacttgttgactggtctgatattcgtgagcaaaaacgacttatggtattgcgagcttcagtcgcactacacggtcgttctgttactctttatgagaaagcgttcccgctttcagagcaatgttcaaagaaagctcatgaccaatttctagccgaccttgcgagcattctaccgagtaacaccacaccgctcattgtcagtgatgctggctttaaagtgccatggtataaatccgttgagaagctgggttggtactggttaagtcgagtaagaggaaaagtacaatatgcagacctaggagcggaaaactggaaacctatcagcaacttacatgatatgtcatctagtcactcaaagactttaggctataagaggctgactaaaagcaatccaatctcatgccaaattctattgtataaatctcgctctaaaggccgaaaaaatcagcgctcgacacggactcattgtcaccacccgtcacctaaaatctactcagcgtcggcaaaggagccatgggttctagcaactaacttacctgttgaaattcgaacacccaaacaacttgttaatatctattcgaagcgaatgcagattgaagaaaccttccgagacttgaaaagtcctgcctacggactaggcctacgccatagccgaacgagcagctcagagcgttttgatatcatgctgctaatcgccctgatgcttcaactaacatgttggcttgcgggcgttcatgctcagaaacaaggttgggacaagcacttccaggctaacacagtcagaaatcgaaacgtactctcaacagttcgcttaggcatggaagttttgcggcattctggctacacaataacaagggaagacttactcgtggctgcaaccctactagctcaaaatttattcacacatggttacgctttggggaaattatgaggggatcgctctagagcgatccgggatctcgggaaaagcgttggtgaccaaaggtgccttttatcatcactttaaaaataaaaaacaattactcagtgcctgttataagcagcaattaattatgattgatgcctacatcacaacaaaaactgatttaacaaatggttggtctgccttagaaagtatatttgaacattatcttgattatattattgataataataaaaaccttatccctatccaagaagtgatgcctatcattggttggaatgaacttgaaaaaaattagccttgaatacattactggtaaggtaaacgccattgtcagcaaattgatccaagagaaccaacttaaagctttcctgacggaatgttaattctcgttgaccctgagcactgatgaatcccctaatgattttggtaaaaatcattaagttaaggtggatacacatcttgtcatatgatcccggtaatgtgagttagctcactcattaggcaccccaggctttacactttatgcttccggctcgtatgttgtgtggaattgtgagcggataacaatttcacacaggaaacagctatgaccatgattacgccaagcgcgcaattaaccctcactaaagggaacaaaagctggagctccaccgcggtggcggccgctctagaactagtggatcccccgggctgcaggaattcgatatcaagcttatcgataccgctgacctcgagcatcagattggctattggccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgtcaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtcttcaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccgcggccgggaacggtgcattggaacgcggattccccgtgccaagagtgacgtaagtaccgcctatagactctataggcacacccctttggctcttatgcatgctatactgtttttggcttggggcctatacacccccgcttccttatgctataggtgatggtatagcttagcctataggtgtgggttattgaccattattgaccactcccctattggtgacgatactttccattactaatccataacatggctctttgccacaactatctctattggctatatgccaatactctgtccttcagagactgacacggactctgtatttttacaggatggggtcccatttattatttacaaattcacatatacaacaacgccgtcccccgtgcccgcagtttttattaaacatagcgtgggatctccacgcgaatctcgggtacgtgttccggacatgggctcttctccggtagcggcggagcttccacatccgagccctggtcccatgcctccagcggctcatggtcgctcggcagctccttgctcctaacagtggaggccagacttaggcacagcacaatgcccaccaccaccagtgtgccgcacaaggccgtggcggtagggtatgtgtctgaaaatgagcgtggagattgggctcgcacggctgacgcagatggaagacttaaggcagcggcagaagaagatgcaggcagctgagttgttgtattctgataagagtcagaggtaactcccgttgcggtgctgttaacggtggagggcagtgtagtctgagcagtactcgttgctgccgcgcgcgccaccagacataatagctgacagactaacagactgttcctttccatgggtcttttctgcagtcaccgtcggatcaatcattcatctcgtgacttcttcgtgtgtggtgtttacctatatatctaaatttaatatttcgtttattaaaatttaatatatttcgacgatgaatttctcaaggatatttttcttcgtgttcgctttggttctggctttgtcaacagtttcggctgcgccagagccgaaaggtacccaggtgcagctgcaggagtcggggggaggcttggtaaagccgggggggtcccttagagtctcctgtgcagcctctggattcactttcagaaacgcctggatgagctgggtccgccaggctccagggaaggggctggagtgggtcggccgtattaaaagcaaaattgatggtgggacaacagactatgctgcacccgtgaaaggcagattcaccatctcaagagatgattcaaaaaacacgttatatctgcaaatgaatagcctgaaagccgaggacacagccgtatattactgtaccacggggattatgataacatttgggggagttatccctcccccgaattggggccagggaaccctggtcaccgtctcctcagcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacacctttccggctgtcctacagtcctcaggactctacttccttagcaacgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaagcgccagagccgaagctttcctatgagctgacacagccaccctcggtgtcagtgtccccaggacaaacggccaggatcacctgctctggagatgcattgccagaaaaatatgtttattggtaccagcagaagtcaggccaggcccctgtggtggtcatctatgaggacagcaaacgaccctccgggatccctgagagattctctggctccagctcagggacaatggccaccttgactatcagtggggcccaggtggaagatgaaggtgactactactgttactcaactgacagcagtggttatcatagggaggtgttcagcggagggaccaagctgaccgtcctaggtcagcccaaggctgccccctcggtcactctgttcccaccctcctctgaggagcttcaagccaacaaggccacactggtgtgtctcataagtgactcctacccgggagccgtgacagtggcctggaaggcagatagcagccccgtcaaggcgggagtggagaccaccacaccctccaaacaaagcaacaacaagtacgcggccagcagctacctgagcctgacgcttgagcagtggaagtcccacaaaagctacagctgccaggtcacgcatgaagggagcaccgtggagaagacagtggcccctgcagaatgttcaccgcggagggagggaagggccctttttgaagggggaggaaacttcgcgccatgactcctctcgtgccccccgcacggaacactgatgtgcagagggccctctgccattgctgcttcctctgcccttcctcgtcactctgaatgtggcttctttgctactgccacagcaagaaataaaatctcaacatctaaatgggtttcctgagatttttcaagagtcgttaagcacattccttccccagcaccccttgctgcaggccagtgccaggcaccaacttggctactgctgcccatgagagaaatccagttcaatattttccaaagcaaaatggattacatatgccctagatcctgattaacaggtgttttgtattatctgtgctttcgcttcacccacattatcccattgcctcccctcgagggggggcccggtacccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactactccgggatcatatgacaagatgtgtatccaccttaacttaatgatttttaccaaaatcattaggggattcatcagtgctcagggtcaacgagaattaacattccgtcaggaaagcttatgatgatgatgtgcttaaaaacttactcaatggctggttatgcatatcgcaatacatgcgaaaaacctaaaagagcttgccgataaaaaaggccaatttattgctatttaccgcggctttttattgagcttgaaagataaataaaatagataggttttatttgaagctaaatcttctttatcgtaaaaaatgccctcttgggttatcaagagggtcattatatttcgcggaataacatcatttggtgacgaaataactaagcacttgtctcctgtttactcccctgagcttgaggggttaacatgaaggtcatcgatagcaggataataatacagtaaaacgctaaaccaataatccaaatccagccatcccaaattggtagtgaatgattataaataacagcaaacagtaatgggccaataacaccggttgcattggtaaggctcaccaataatccctgtaaagcaccttgctgatgactctttgtttggatagacatcactccctgtaatgcaggtaaagcgatcccaccaccagccaataaaattaaaacagggaaaactaaccaaccttcagatataaacgctaaaaaggcaaatgcactactatctgcaataaatccgagcagtactgccgttttttcgcccatttagtggctattcttcctgccacaaaggcttggaatactgagtgtaaaagaccaagacccgtaatgaaaagccaaccatcatgctattcatcatcacgatttctgtaatagcaccacaccgtgctggattggctatcaatgcgctgaaataataatcaacaaatggcatcgttaaataagtgatgtataccgatcagcttttgttccctttagtgagggttaattgcgcgcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccac

SEQ ID NO: 101 - DNA, 11593 bp, ARTIFICIAL SEQUENCE
  Synthetic
ctgacgcgccctgtagcggcgcattaagcgcggcgggtgtggtggttacgcgcagcgtgaccgctacacttgccagcgccctagcgcccgctcctttcgctttcttcccttcctttctcgccacgttcgccggcatcagattggctattggccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccgcggccgggaacggtgcattggaacgcggattccccgtgccaagagtgacgtaagtaccgcctatagactctataggcacacccctttggctcttatgcatgctatactgtttttggcttggggcctatacacccccgcttccttatgctataggtgatggtatagcttagcctataggtgtgggttattgaccattattgaccactcccctattggtgacgatactttccattactaatccataacatggctctttgccacaactatctctattggctatatgccaatactctgtccttcagagactgacacggactctgtatttttacaggatggggtcccatttattatttacaaattcacatatacaacaacgccgtcccccgtgcccgcagtttttattaaacatagcgtgggatctccacgcgaatctcgggtacgtgttccggacatgggctcttctccggtagcggcggagcttccacatccgagccctggtcccatgcctccagcggctcatggtcgctcggcagctccttgctcctaacagtggaggccagacttaggcacagcacaatgcccaccaccaccagtgtgccgcacaaggccgtggcggtagggtatgtgtctgaaaatgagcgtggagattgggctcgcacggctgacgcagatggaagacttaaggcagcggcagaagaagatgcaggcagctgagttgttgtattctgataagagtcagaggtaactcccgttgcggtgctgttaacggtggagggcagtgtagtctgagcagtactcgttgctgccgcgcgcgccaccagacataatagctgacagactaacagactgttcctttccatgggtcttttctgcagtcaccgtcggaccatgtgcgaactcgatattttacacgactctctttaccaattctgccccgaattacacttaaaacgactcaacagcttaacgttggcttgccacgcattacttgactgtaaaactctcactcttaccgaacttggccgtaacctgccaaccaaagcgagaacaaaacataacatcaaacgaatcgaccgattgttaggtaatcgtcacctccacaaagagcgactcgctgtataccgttggcatgctagctttatctgttcgggcaatacgatgcccattgtacttgttgactggtctgatattcgtgagcaaaaacgacttatggtattgcgagcttcagtcgcactacacggtcgttctgttactctttatgagaaagcgttcccgctttcagagcaatgttcaaagaaagctcatgaccaatttctagccgaccttgcgagcattctaccgagtaacaccacaccgctcattgtcagtgatgctggctttaaagtgccatggtataaatccgttgagaagctgggttggtactggttaagtcgagtaagaggaaaagtacaatatgcagacctaggagcggaaaactggaaacctatcagcaacttacatgatatgtcatctagtcactcaaagactttaggctataagaggctgactaaaagcaatccaatctcatgccaaattctattgtataaatctcgctctaaaggccgaaaaaatcagcgctcgacacggactcattgtcaccacccgtcacctaaaatctactcagcgtcggcaaaggagccatgggttctagcaactaacttacctgttgaaattcgaacacccaaacaacttgttaatatctattcgaagcgaatgcagattgaagaaaccttccgagacttgaaaagtcctgcctacggactaggcctacgccatagccgaacgagcagctcagagcgttttgatatcatgctgctaatcgccctgatgcttcaactaacatgttggcttgcgggcgttcatgctcagaaacaaggttgggacaagcacttccaggctaacacagtcagaaatcgaaacgtactctcaacagttcgcttaggcatggaagttttgcggcattctggctacacaataacaagggaagacttactcgtggctgcaaccctactagctcaaaatttattcacacatggttacgctttggggaaattatgaggggatcgctctagagcgatccgggatctcgggaaaagcgttggtgaccaaaggtgccttttatcatcactttaaaaataaaaaacaattactcagtgcctgttataagcagcaattaattatgattgatgcctacatcacaacaaaaactgatttaacaaatggttggtctgccttagaaagtatatttgaacattatcttgattatattattgataataataaaaaccttatccctatccaagaagtgatgcctatcattggttggaatgaacttgaaaaaaattagccttgaatacattactggtaaggtaaacgccattgtcagcaaattgatccaagagaaccaacttaaagctttcctgacggaatgttaattctcgttgaccctgagcactgatgaatcccctaatgattttggtaaaaatcattaagttaaggtggatacacatcttgtcatatgatcccggtaatgtgagttagctcactcattaggcaccccaggctttacactttatgcttccggctcgtatgttgtgtggaattgtgagcggataacaatttcacacaggaaacagctatgaccatgattacgccaagcgcgcaattaaccctcactaaagggaacaaaagctggagctccaccgcggtggcggccgctctagaactagtggatcccccgggctgcaggaattcgatatcaagcttatcgataccgctgacctcgagcatcagattggctattggccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgtcaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtcttcaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccgcggccgggaacggtgcattggaacgcggattccccgtgccaagagtgacgtaagtaccgcctatagactctataggcacacccctttggctcttatgcatgctatactgtttttggcttggggcctatacacccccgcttccttatgctataggtgatggtatagcttagcctataggtgtgggttattgaccattattgaccactcccctattggtgacgatactttccattactaatccataacatggctctttgccacaactatctctattggctatatgccaatactctgtccttcagagactgacacggactctgtatttttacaggatggggtcccatttattatttacaaattcacatatacaacaacgccgtcccccgtgcccgcagtttttattaaacatagcgtgggatctccacgcgaatctcgggtacgtgttccggacatgggctcttctccggtagcggcggagcttccacatccgagccctggtcccatgcctccagcggctcatggtcgctcggcagctccttgctcctaacagtggaggccagacttaggcacagcacaatgcccaccaccaccagtgtgccgcacaaggccgtggcggtagggtatgtgtctgaaaatgagcgtggagattgggctcgcacggctgacgcagatggaagacttaaggcagcggcagaagaagatgcaggcagctgagttgttgtattctgataagagtcagaggtaactcccgttgcggtgctgttaacggtggagggcagtgtagtctgagcagtactcgttgctgccgcgcgcgccaccagacataatagctgacagactaacagactgttcctttccatgggtcttttctgcagtcaccgtcggatcaatcattcatctcgtgacttcttcgtgtgtggtgtttacctatatatctaaatttaatatttcgtttattaaaatttaatatatttcgacgatgaatttctcaaggatatttttcttcgtgttcgctttggttctggctttgtcaacagtttcggctgcgccagagccgaaaggtacccaggtgcagctgcaggagtcggggggaggcttggtaaagccgggggggtcccttagagtctcctgtgcagcctctggattcactttcagaaacgcctggatgagctgggtccgccaggctccagggaaggggctggagtgggtcggccgtattaaaagcaaaattgatggtgggacaacagactatgctgcacccgtgaaaggcagattcaccatctcaagagatgattcaaaaaacacgttatatctgcaaatgaatagcctgaaagccgaggacacagccgtatattactgtaccacggggattatgataacatttgggggagttatccctcccccgaattggggccagggaaccctggtcaccgtctcctcagcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacacctttccggctgtcctacagtcctcaggactctacttccttagcaacgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaagcgccagagccgaaaaagctttcctatgagctgacacagccaccctcggtgtcagtgtccccaggacaaacggccaggatcacctgctctggagatgcattgccagaaaaatatgtttattggtaccagcagaagtcaggccaggcccctgtggtggtcatctatgaggacagcaaacgaccctccgggatccctgagagattctctggctccagctcagggacaatggccaccttgactatcagtggggcccaggtggaagatgaaggtgactactactgttactcaactgacagcagtggttatcatagggaggtgttcagcggagggaccaagctgaccgtcctaggtcagcccaaggctgccccctcggtcactctgttcccaccctcctctgaggagcttcaagccaacaaggccacactggtgtgtctcataagtgactcctacccgggagccgtgacagtggcctggaaggcagatagcagccccgtcaaggcgggagtggagaccaccacaccctccaaacaaagcaacaacaagtacgcggccagcagctacctgagcctgacgcttgagcagtggaagtcccacaaaagctacagctgccaggtcacgcatgaagggagcaccgtggagaagacagtggcccctgcagaatgttcaccgcggagggagggaagggccctttttgaagggggaggaaacttcgcgccatgactcctctcgtgccccccgcacggaacactgatgtgcagagggccctctgccattgctgcttcctctgcccttcctcgtcactctgaatgtggcttctttgctactgccacagcaagaaataaaatctcaacatctaaatgggtttcctgagatttttcaagagtcgttaagcacattccttccccagcaccccttgctgcaggccagtgccaggcaccaacttggctactgctgcccatgagagaaatccagttcaatattttccaaagcaaaatggattacatatgccctagatcctgattaacaggtgttttgtattatctgtgctttcgcttcacccacattatcccattgcctcccctcgagggggggcccggtacccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactactccgggatcatatgacaagatgtgtatccaccttaacttaatgatttttaccaaaatcattaggggattcatcagtgctcagggtcaacgagaattaacattccgtcaggaaagcttatgatgatgatgtgcttaaaaacttactcaatggctggttatgcatatcgcaatacatgcgaaaaacctaaaagagcttgccgataaaaaaggccaatttattgctatttaccgcggctttttattgagcttgaaagataaataaaatagataggttttatttgaagctaaatcttctttatcgtaaaaaatgccctcttgggttatcaagagggtcattatatttcgcggaataacatcatttggtgacgaaataactaagcacttgtctcctgtttactcccctgagcttgaggggttaacatgaaggtcatcgatagcaggataataatacagtaaaacgctaaaccaataatccaaatccagccatcccaaattggtagtgaatgattataaataacagcaaacagtaatgggccaataacaccggttgcattggtaaggctcaccaataatccctgtaaagcaccttgctgatgactctttgtttggatagacatcactccctgtaatgcaggtaaagcgatcccaccaccagccaataaaattaaaacagggaaaactaaccaaccttcagatataaacgctaaaaaggcaaatgcactactatctgcaataaatccgagcagtactgccgttttttcgcccatttagtggctattcttcctgccacaaaggcttggaatactgagtgtaaaagaccaagacccgtaatgaaaagccaaccatcatgctattcatcatcacgatttctgtaatagcaccacaccgtgctggattggctatcaatgcgctgaaataataatcaacaaatggcatcgttaaataagtgatgtataccgatcagcttttgttccctttagtgagggttaattgcgcgcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccac

SEQ ID NO: 102 - DNA, 12339 bp, ARTIFICIAL SEQUENCE
  Synthetic
ctgacgcgccctgtagcggcgcattaagcgcggcgggtgtggtggttacgcgcagcgtgaccgctacacttgccagcgccctagcgcccgctcctttcgctttcttcccttcctttctcgccacgttcgccggcatcagattggctattggccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccgcggccgggaacggtgcattggaacgcggattccccgtgccaagagtgacgtaagtaccgcctatagactctataggcacacccctttggctcttatgcatgctatactgtttttggcttggggcctatacacccccgcttccttatgctataggtgatggtatagcttagcctataggtgtgggttattgaccattattgaccactcccctattggtgacgatactttccattactaatccataacatggctctttgccacaactatctctattggctatatgccaatactctgtccttcagagactgacacggactctgtatttttacaggatggggtcccatttattatttacaaattcacatatacaacaacgccgtcccccgtgcccgcagtttttattaaacatagcgtgggatctccacgcgaatctcgggtacgtgttccggacatgggctcttctccggtagcggcggagcttccacatccgagccctggtcccatgcctccagcggctcatggtcgctcggcagctccttgctcctaacagtggaggccagacttaggcacagcacaatgcccaccaccaccagtgtgccgcacaaggccgtggcggtagggtatgtgtctgaaaatgagcgtggagattgggctcgcacggctgacgcagatggaagacttaaggcagcggcagaagaagatgcaggcagctgagttgttgtattctgataagagtcagaggtaactcccgttgcggtgctgttaacggtggagggcagtgtagtctgagcagtactcgttgctgccgcgcgcgccaccagacataatagctgacagactaacagactgttcctttccatgggtcttttctgcagtcaccgtcggaccatgtgtgaacttgatattttacatgattctctttaccaattctgccccgaattacacttaaaacgactcaacagcttaacgttggcttgccacgcattacttgactgtaaaactctcactcttaccgaacttggccgtaacctgccaaccaaagcgagaacaaaacataacatcaaacgaatcgaccgattgttaggtaatcgtcacctccacaaagagcgactcgctgtataccgttggcatgctagctttatctgttcgggcaatacgatgcccattgtacttgttgactggtctgatattcgtgagcaaaaacgacttatggtattgcgagcttcagtcgcactacacggtcgttctgttactctttatgagaaagcgttcccgctttcagagcaatgttcaaagaaagctcatgaccaatttctagccgaccttgcgagcattctaccgagtaacaccacaccgctcattgtcagtgatgctggctttaaagtgccatggtataaatccgttgagaagctgggttggtactggttaagtcgagtaagaggaaaagtacaatatgcagacctaggagcggaaaactggaaacctatcagcaacttacatgatatgtcatctagtcactcaaagactttaggctataagaggctgactaaaagcaatccaatctcatgccaaattctattgtataaatctcgctctaaaggccgaaaaaatcagcgctcgacacggactcattgtcaccacccgtcacctaaaatctactcagcgtcggcaaaggagccatgggttctagcaactaacttacctgttgaaattcgaacacccaaacaacttgttaatatctattcgaagcgaatgcagattgaagaaaccttccgagacttgaaaagtcctgcctacggactaggcctacgccatagccgaacgagcagctcagagcgttttgatatcatgctgctaatcgccctgatgcttcaactaacatgttggcttgcgggcgttcatgctcagaaacaaggttgggacaagcacttccaggctaacacagtcagaaatcgaaacgtactctcaacagttcgcttaggcatggaagttttgcggcattctggctacacaataacaagggaagacttactcgtggctgcaaccctactagctcaaaatttattcacacatggttacgctttggggaaattatgataatgatccagatcacttctggctaataaaagatcagagctctagagatctgtgtgttggttttttgtggatctgctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcagcacagcaagggggaggattgggaagacaatagcaggcatgctggggatgcggtgggctctatgggtacctctctctctctctctctctctctctctctctctctctctcggtacctctctctctctctctctctctctctctctctctctctctcggtaccaggtgctgaagaattgacccggtgaccaaaggtgccttttatcatcactttaaaaataaaaaacaattactcagtgcctgttataagcagcaattaattatgattgatgcctacatcacaacaaaaactgatttaacaaatggttggtctgccttagaaagtatatttgaacattatcttgattatattattgataataataaaaaccttatccctatccaagaagtgatgcctatcattggttggaatgaacttgaaaaaaattagccttgaatacattactggtaaggtaaacgccattgtcagcaaattgatccaagagaaccaacttaaagctttcctgacggaatgttaattctcgttgaccctgagcactgatgaatcccctaatgattttggtaaaaatcattaagttaaggtggatacacatcttgtcatatgatcccggtaatgtgagttagctcactcattaggcaccccaggctttacactttatgcttccggctcgtatgttgtgtggaattgtgagcggataacaatttcacacaggaaacagctatgaccatgattacgccaagcgcgcaattaaccctcactaaagggaacaaaagctggagctccaccgcggtggcggccgctctagaactagtggatcccccgggctgcaggaattcgatatcaagcttatcgataccgctgacctcgagctgcagaaaaatgccaggtggactatgaactcacatccaaaggagcttgacctgatacctgattttcttcaaacaggggaaacaacacaatcccacaaaacagctcagagagaaaccatcactgatggctacagcaccaaggtatgcaatggcaatccattcgacattcatctgtgacctgagcaaaatgatttctctctccatgaatggttgcttctttccctcatgaaaaggcaatttccacactcacaatatgcgacaaagacaaacagagaacaattaatgtgctccttcctaatgtcaaaattgtagtggcaaagaggagaacaaaatctcaagttctgagtaggttttagtgattggataagaggctttgacctgtgagctcacctggacttcatatccttttggataaaaagtgcttttataactttcaggtctccgagtctttattcatgagactgttggtttagggacagacccacaatgaaatgcctggcataggaaagggcagcagagccttagctgaccttttcttgggacaagcattgtcaaacaatgtgtgacaaaactatttgtactgctttgcacagctgtgctgggcagggcgatccattgccacctatcccaggtaaccttccaactgcaagaagattgttgcttactctctctagaaagcttctgcagactgacatgcatttcataggtagagataacatttactgggaagcacatctatcatcacaaaaagcaggcaagattttcagactttcttagtggctgaaatagaagcaaaagacgtaattaaaaacaaaatgaaacaaaaaaaatcagttgatacctgtggtgtagacatccagcaaaaaaatattatttgcactaccatcttgtcttaagtcctcagacttagcaaggagaatgtagatttccacagtatatatgttttcacaaaaggaaggagagaaacaaaagaaaatggcactgactaaacttcagctagtggtataggaaagtaattctgcttaacagagattgcagtgatctctatgtatgtcctgaagaattatgttgtacttttttcccccatttttaaatcaaacagtgctttacagaggtcagaatggtttctttactgtttgtcaattctattatttcaatacagaacaatagcttctataactgaaatatatttgctattgtatattatgattgtccctcgaaccatgaacactcctccagctgaatttcacaattcctctgtcatctgccaggccattaagttattcatggaagatctttgaggaacactgcaagttcatatcataaacacatttgaaattgagtattggtttgcattgtatggagctatgttttgctgtatcctcagaaaaaaagtttgttataaagcattcacacccataaaaagatagatttaaatattccagctataggaaagaaagtgcgtctgctcttcactctagtctcagttggctccttcacatgcatgcttctttatttctcctattttgtcaagaaaataataggtcacgtcttgttctcacttatgtcctgcctagcatggctcagatgcacgttgtagatacaagaaggatcaaatgaaacagacttctggtctgttacctacaaccatagtaataagcacactaactaataattgctaattatgttttccatctctaaggttcccatatttttctgttttcttaaagatcccattatctggttgtaactgaagctcaatggaacatgagcaatatttcccagtcttctctcccatccaacagtcctgatggattagcagaacaggcagaaaacacattgttacccagaattaaaaactaatatttgctctccattcaatccaaaatggacctattgaaactaaaatctaacccaatcccattaaatgatttctatggtgtcaaaggtcaaacttctgaagggaacctgtgggtgggtcacaattcaggctatatattccccagggctcagccagtggatcaatcattcatctcgtgacttcttcgtgtgtggtgtttacctatatatctaaatttaatatttcgtttattaaaatttaatatatttcgacgatgaatttctcaaggatatttttcttcgtgttcgctttggttctggctttgtcaacagtttcggctgcgccagagccgaaaggtacccaggtgcagctgcaggagtcggggggaggcttggtaaagccgggggggtcccttagagtctcctgtgcagcctctggattcactttcagaaacgcctggatgagctgggtccgccaggctccagggaaggggctggagtgggtcggccgtattaaaagcaaaattgatggtgggacaacagactatgctgcacccgtgaaaggcagattcaccatctcaagagatgattcaaaaaacacgttatatctgcaaatgaatagcctgaaagccgaggacacagccgtatattactgtaccacggggattatgataacatttgggggagttatccctcccccgaattggggccagggaaccctggtcaccgtctcctcagcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacacctttccggctgtcctacagtcctcaggactctacttccttagcaacgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaagcgccagagccgaagctttcctatgagctgacacagccaccctcggtgtcagtgtccccaggacaaacggccaggatcacctgctctggagatgcattgccagaaaaatatgtttattggtaccagcagaagtcaggccaggcccctgtggtggtcatctatgaggacagcaaacgaccctccgggatccctgagagattctctggctccagctcagggacaatggccaccttgactatcagtggggcccaggtggaagatgaaggtgactactactgttactcaactgacagcagtggttatcatagggaggtgttcagcggagggaccaagctgaccgtcctaggtcagcccaaggctgccccctcggtcactctgttcccaccctcctctgaggagcttcaagccaacaaggccacactggtgtgtctcataagtgactcctacccgggagccgtgacagtggcctggaaggcagatagcagccccgtcaaggcgggagtggagaccaccacaccctccaaacaaagcaacaacaagtacgcggccagcagctacctgagcctgacgcttgagcagtggaagtcccacaaaagctacagctgccaggtcacgcatgaagggagcaccgtggagaagacagtggcccctgcagaatgttcaccgcggagggagggaagggccctttttgaagggggaggaaacttcgcgccatgactcctctcgtgccccccgcacggaacactgatgtgcagagggccctctgccattgctgcttcctctgcccttcctcgtcactctgaatgtggcttctttgctactgccacagcaagaaataaaatctcaacatctaaatgggtttcctgagatttttcaagagtcgttaagcacattccttccccagcaccccttgctgcaggccagtgccaggcaccaacttggctactgctgcccatgagagaaatccagttcaatattttccaaagcaaaatggattacatatgccctagatcctgattaacaggtgttttgtattatctgtgctttcgcttcacccacattatcccattgcctcccctcgagggggggcccggtacccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactactccgggatcatatgacaagatgtgtatccaccttaacttaatgatttttaccaaaatcattaggggattcatcagtgctcagggtcaacgagaattaacattccgtcaggaaagcttatgatgatgatgtgcttaaaaacttactcaatggctggttatgcatatcgcaatacatgcgaaaaacctaaaagagcttgccgataaaaaaggccaatttattgctatttaccgcggctttttattgagcttgaaagataaataaaatagataggttttatttgaagctaaatcttctttatcgtaaaaaatgccctcttgggttatcaagagggtcattatatttcgcggaataacatcatttggtgacgaaataactaagcacttgtctcctgtttactcccctgagcttgaggggttaacatgaaggtcatcgatagcaggataataatacagtaaaacgctaaaccaataatccaaatccagccatcccaaattggtagtgaatgattataaataacagcaaacagtaatgggccaataacaccggttgcattggtaaggctcaccaataatccctgtaaagcaccttgctgatgactctttgtttggatagacatcactccctgtaatgcaggtaaagcgatcccaccaccagccaataaaattaaaacagggaaaactaaccaaccttcagatataaacgctaaaaaggcaaatgcactactatctgcaataaatccgagcagtactgccgttttttcgcccatttagtggctattcttcctgccacaaaggcttggaatactgagtgtaaaagaccaagacccgtaatgaaaagccaaccatcatgctattcatcatcacgatttctgtaatagcaccacaccgtgctggattggctatcaatgcgctgaaataataatcaacaaatggcatcgttaaataagtgatgtataccgatcagcttttgttccctttagtgagggttaattgcgcgcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccac

SEQ ID NO: 103 - DNA, 12342 bp, ARTIFICIAL SEQUENCE
  Synthetic
ctgacgcgccctgtagcggcgcattaagcgcggcgggtgtggtggttacgcgcagcgtgaccgctacacttgccagcgccctagcgcccgctcctttcgctttcttcccttcctttctcgccacgttcgccggcatcagattggctattggccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccgcggccgggaacggtgcattggaacgcggattccccgtgccaagagtgacgtaagtaccgcctatagactctataggcacacccctttggctcttatgcatgctatactgtttttggcttggggcctatacacccccgcttccttatgctataggtgatggtatagcttagcctataggtgtgggttattgaccattattgaccactcccctattggtgacgatactttccattactaatccataacatggctctttgccacaactatctctattggctatatgccaatactctgtccttcagagactgacacggactctgtatttttacaggatggggtcccatttattatttacaaattcacatatacaacaacgccgtcccccgtgcccgcagtttttattaaacatagcgtgggatctccacgcgaatctcgggtacgtgttccggacatgggctcttctccggtagcggcggagcttccacatccgagccctggtcccatgcctccagcggctcatggtcgctcggcagctccttgctcctaacagtggaggccagacttaggcacagcacaatgcccaccaccaccagtgtgccgcacaaggccgtggcggtagggtatgtgtctgaaaatgagcgtggagattgggctcgcacggctgacgcagatggaagacttaaggcagcggcagaagaagatgcaggcagctgagttgttgtattctgataagagtcagaggtaactcccgttgcggtgctgttaacggtggagggcagtgtagtctgagcagtactcgttgctgccgcgcgcgccaccagacataatagctgacagactaacagactgttcctttccatgggtcttttctgcagtcaccgtcggaccatgtgtgaacttgatattttacatgattctctttaccaattctgccccgaattacacttaaaacgactcaacagcttaacgttggcttgccacgcattacttgactgtaaaactctcactcttaccgaacttggccgtaacctgccaaccaaagcgagaacaaaacataacatcaaacgaatcgaccgattgttaggtaatcgtcacctccacaaagagcgactcgctgtataccgttggcatgctagctttatctgttcgggcaatacgatgcccattgtacttgttgactggtctgatattcgtgagcaaaaacgacttatggtattgcgagcttcagtcgcactacacggtcgttctgttactctttatgagaaagcgttcccgctttcagagcaatgttcaaagaaagctcatgaccaatttctagccgaccttgcgagcattctaccgagtaacaccacaccgctcattgtcagtgatgctggctttaaagtgccatggtataaatccgttgagaagctgggttggtactggttaagtcgagtaagaggaaaagtacaatatgcagacctaggagcggaaaactggaaacctatcagcaacttacatgatatgtcatctagtcactcaaagactttaggctataagaggctgactaaaagcaatccaatctcatgccaaattctattgtataaatctcgctctaaaggccgaaaaaatcagcgctcgacacggactcattgtcaccacccgtcacctaaaatctactcagcgtcggcaaaggagccatgggttctagcaactaacttacctgttgaaattcgaacacccaaacaacttgttaatatctattcgaagcgaatgcagattgaagaaaccttccgagacttgaaaagtcctgcctacggactaggcctacgccatagccgaacgagcagctcagagcgttttgatatcatgctgctaatcgccctgatgcttcaactaacatgttggcttgcgggcgttcatgctcagaaacaaggttgggacaagcacttccaggctaacacagtcagaaatcgaaacgtactctcaacagttcgcttaggcatggaagttttgcggcattctggctacacaataacaagggaagacttactcgtggctgcaaccctactagctcaaaatttattcacacatggttacgctttggggaaattatgataatgatccagatcacttctggctaataaaagatcagagctctagagatctgtgtgttggttttttgtggatctgctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcagcacagcaagggggaggattgggaagacaatagcaggcatgctggggatgcggtgggctctatgggtacctctctctctctctctctctctctctctctctctctctctcggtacctctctctctctctctctctctctctctctctctctctctcggtaccaggtgctgaagaattgacccggtgaccaaaggtgccttttatcatcactttaaaaataaaaaacaattactcagtgcctgttataagcagcaattaattatgattgatgcctacatcacaacaaaaactgatttaacaaatggttggtctgccttagaaagtatatttgaacattatcttgattatattattgataataataaaaaccttatccctatccaagaagtgatgcctatcattggttggaatgaacttgaaaaaaattagccttgaatacattactggtaaggtaaacgccattgtcagcaaattgatccaagagaaccaacttaaagctttcctgacggaatgttaattctcgttgaccctgagcactgatgaatcccctaatgattttggtaaaaatcattaagttaaggtggatacacatcttgtcatatgatcccggtaatgtgagttagctcactcattaggcaccccaggctttacactttatgcttccggctcgtatgttgtgtggaattgtgagcggataacaatttcacacaggaaacagctatgaccatgattacgccaagcgcgcaattaaccctcactaaagggaacaaaagctggagctccaccgcggtggcggccgctctagaactagtggatcccccgggctgcaggaattcgatatcaagcttatcgataccgctgacctcgagctgcagaaaaatgccaggtggactatgaactcacatccaaaggagcttgacctgatacctgattttcttcaaacaggggaaacaacacaatcccacaaaacagctcagagagaaaccatcactgatggctacagcaccaaggtatgcaatggcaatccattcgacattcatctgtgacctgagcaaaatgatttctctctccatgaatggttgcttctttccctcatgaaaaggcaatttccacactcacaatatgcgacaaagacaaacagagaacaattaatgtgctccttcctaatgtcaaaattgtagtggcaaagaggagaacaaaatctcaagttctgagtaggttttagtgattggataagaggctttgacctgtgagctcacctggacttcatatccttttggataaaaagtgcttttataactttcaggtctccgagtctttattcatgagactgttggtttagggacagacccacaatgaaatgcctggcataggaaagggcagcagagccttagctgaccttttcttgggacaagcattgtcaaacaatgtgtgacaaaactatttgtactgctttgcacagctgtgctgggcagggcgatccattgccacctatcccaggtaaccttccaactgcaagaagattgttgcttactctctctagaaagcttctgcagactgacatgcatttcataggtagagataacatttactgggaagcacatctatcatcacaaaaagcaggcaagattttcagactttcttagtggctgaaatagaagcaaaagacgtaattaaaaacaaaatgaaacaaaaaaaatcagttgatacctgtggtgtagacatccagcaaaaaaatattatttgcactaccatcttgtcttaagtcctcagacttagcaaggagaatgtagatttccacagtatatatgttttcacaaaaggaaggagagaaacaaaagaaaatggcactgactaaacttcagctagtggtataggaaagtaattctgcttaacagagattgcagtgatctctatgtatgtcctgaagaattatgttgtacttttttcccccatttttaaatcaaacagtgctttacagaggtcagaatggtttctttactgtttgtcaattctattatttcaatacagaacaatagcttctataactgaaatatatttgctattgtatattatgattgtccctcgaaccatgaacactcctccagctgaatttcacaattcctctgtcatctgccaggccattaagttattcatggaagatctttgaggaacactgcaagttcatatcataaacacatttgaaattgagtattggtttgcattgtatggagctatgttttgctgtatcctcagaaaaaaagtttgttataaagcattcacacccataaaaagatagatttaaatattccagctataggaaagaaagtgcgtctgctcttcactctagtctcagttggctccttcacatgcatgcttctttatttctcctattttgtcaagaaaataataggtcacgtcttgttctcacttatgtcctgcctagcatggctcagatgcacgttgtagatacaagaaggatcaaatgaaacagacttctggtctgttacctacaaccatagtaataagcacactaactaataattgctaattatgttttccatctctaaggttcccatatttttctgttttcttaaagatcccattatctggttgtaactgaagctcaatggaacatgagcaatatttcccagtcttctctcccatccaacagtcctgatggattagcagaacaggcagaaaacacattgttacccagaattaaaaactaatatttgctctccattcaatccaaaatggacctattgaaactaaaatctaacccaatcccattaaatgatttctatggtgtcaaaggtcaaacttctgaagggaacctgtgggtgggtcacaattcaggctatatattccccagggctcagccagtggatcaatcattcatctcgtgacttcttcgtgtgtggtgtttacctatatatctaaatttaatatttcgtttattaaaatttaatatatttcgacgatgaatttctcaaggatatttttcttcgtgttcgctttggttctggctttgtcaacagtttcggctgcgccagagccgaaaggtacccaggtgcagctgcaggagtcggggggaggcttggtaaagccgggggggtcccttagagtctcctgtgcagcctctggattcactttcagaaacgcctggatgagctgggtccgccaggctccagggaaggggctggagtgggtcggccgtattaaaagcaaaattgatggtgggacaacagactatgctgcacccgtgaaaggcagattcaccatctcaagagatgattcaaaaaacacgttatatctgcaaatgaatagcctgaaagccgaggacacagccgtatattactgtaccacggggattatgataacatttgggggagttatccctcccccgaattggggccagggaaccctggtcaccgtctcctcagcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacacctttccggctgtcctacagtcctcaggactctacttccttagcaacgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaagcgccagagccgaaaaagctttcctatgagctgacacagccaccctcggtgtcagtgtccccaggacaaacggccaggatcacctgctctggagatgcattgccagaaaaatatgtttattggtaccagcagaagtcaggccaggcccctgtggtggtcatctatgaggacagcaaacgaccctccgggatccctgagagattctctggctccagctcagggacaatggccaccttgactatcagtggggcccaggtggaagatgaaggtgactactactgttactcaactgacagcagtggttatcatagggaggtgttcagcggagggaccaagctgaccgtcctaggtcagcccaaggctgccccctcggtcactctgttcccaccctcctctgaggagcttcaagccaacaaggccacactggtgtgtctcataagtgactcctacccgggagccgtgacagtggcctggaaggcagatagcagccccgtcaaggcgggagtggagaccaccacaccctccaaacaaagcaacaacaagtacgcggccagcagctacctgagcctgacgcttgagcagtggaagtcccacaaaagctacagctgccaggtcacgcatgaagggagcaccgtggagaagacagtggcccctgcagaatgttcaccgcggagggagggaagggccctttttgaagggggaggaaacttcgcgccatgactcctctcgtgccccccgcacggaacactgatgtgcagagggccctctgccattgctgcttcctctgcccttcctcgtcactctgaatgtggcttctttgctactgccacagcaagaaataaaatctcaacatctaaatgggtttcctgagatttttcaagagtcgttaagcacattccttccccagcaccccttgctgcaggccagtgccaggcaccaacttggctactgctgcccatgagagaaatccagttcaatattttccaaagcaaaatggattacatatgccctagatcctgattaacaggtgttttgtattatctgtgctttcgcttcacccacattatcccattgcctcccctcgagggggggcccggtacccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactactccgggatcatatgacaagatgtgtatccaccttaacttaatgatttttaccaaaatcattaggggattcatcagtgctcagggtcaacgagaattaacattccgtcaggaaagcttatgatgatgatgtgcttaaaaacttactcaatggctggttatgcatatcgcaatacatgcgaaaaacctaaaagagcttgccgataaaaaaggccaatttattgctatttaccgcggctttttattgagcttgaaagataaataaaatagataggttttatttgaagctaaatcttctttatcgtaaaaaatgccctcttgggttatcaagagggtcattatatttcgcggaataacatcatttggtgacgaaataactaagcacttgtctcctgtttactcccctgagcttgaggggttaacatgaaggtcatcgatagcaggataataatacagtaaaacgctaaaccaataatccaaatccagccatcccaaattggtagtgaatgattataaataacagcaaacagtaatgggccaataacaccggttgcattggtaaggctcaccaataatccctgtaaagcaccttgctgatgactctttgtttggatagacatcactccctgtaatgcaggtaaagcgatcccaccaccagccaataaaattaaaacagggaaaactaaccaaccttcagatataaacgctaaaaaggcaaatgcactactatctgcaataaatccgagcagtactgccgttttttcgcccatttagtggctattcttcctgccacaaaggcttggaatactgagtgtaaaagaccaagacccgtaatgaaaagccaaccatcatgctattcatcatcacgatttctgtaatagcaccacaccgtgctggattggctatcaatgcgctgaaataataatcaacaaatggcatcgttaaataagtgatgtataccgatcagcttttgttccctttagtgagggttaattgcgcgcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccac

SEQ ID NO: 104 - DNA, 11970 bp, ARTIFICIAL SEQUENCE
  Synthetic
ctgacgcgccctgtagcggcgcattaagcgcggcgggtgtggtggttacgcgcagcgtgaccgctacacttgccagcgccctagcgcccgctcctttcgctttcttcccttcctttctcgccacgttcgccggcatcagattggctattggccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccgcggccgggaacggtgcattggaacgcggattccccgtgccaagagtgacgtaagtaccgcctatagactctataggcacacccctttggctcttatgcatgctatactgtttttggcttggggcctatacacccccgcttccttatgctataggtgatggtatagcttagcctataggtgtgggttattgaccattattgaccactcccctattggtgacgatactttccattactaatccataacatggctctttgccacaactatctctattggctatatgccaatactctgtccttcagagactgacacggactctgtatttttacaggatggggtcccatttattatttacaaattcacatatacaacaacgccgtcccccgtgcccgcagtttttattaaacatagcgtgggatctccacgcgaatctcgggtacgtgttccggacatgggctcttctccggtagcggcggagcttccacatccgagccctggtcccatgcctccagcggctcatggtcgctcggcagctccttgctcctaacagtggaggccagacttaggcacagcacaatgcccaccaccaccagtgtgccgcacaaggccgtggcggtagggtatgtgtctgaaaatgagcgtggagattgggctcgcacggctgacgcagatggaagacttaaggcagcggcagaagaagatgcaggcagctgagttgttgtattctgataagagtcagaggtaactcccgttgcggtgctgttaacggtggagggcagtgtagtctgagcagtactcgttgctgccgcgcgcgccaccagacataatagctgacagactaacagactgttcctttccatgggtcttttctgcagtcaccgtcggaccatgtgtgaacttgatattttacatgattctctttaccaattctgccccgaattacacttaaaacgactcaacagcttaacgttggcttgccacgcattacttgactgtaaaactctcactcttaccgaacttggccgtaacctgccaaccaaagcgagaacaaaacataacatcaaacgaatcgaccgattgttaggtaatcgtcacctccacaaagagcgactcgctgtataccgttggcatgctagctttatctgttcgggcaatacgatgcccattgtacttgttgactggtctgatattcgtgagcaaaaacgacttatggtattgcgagcttcagtcgcactacacggtcgttctgttactctttatgagaaagcgttcccgctttcagagcaatgttcaaagaaagctcatgaccaatttctagccgaccttgcgagcattctaccgagtaacaccacaccgctcattgtcagtgatgctggctttaaagtgccatggtataaatccgttgagaagctgggttggtactggttaagtcgagtaagaggaaaagtacaatatgcagacctaggagcggaaaactggaaacctatcagcaacttacatgatatgtcatctagtcactcaaagactttaggctataagaggctgactaaaagcaatccaatctcatgccaaattctattgtataaatctcgctctaaaggccgaaaaaatcagcgctcgacacggactcattgtcaccacccgtcacctaaaatctactcagcgtcggcaaaggagccatgggttctagcaactaacttacctgttgaaattcgaacacccaaacaacttgttaatatctattcgaagcgaatgcagattgaagaaaccttccgagacttgaaaagtcctgcctacggactaggcctacgccatagccgaacgagcagctcagagcgttttgatatcatgctgctaatcgccctgatgcttcaactaacatgttggcttgcgggcgttcatgctcagaaacaaggttgggacaagcacttccaggctaacacagtcagaaatcgaaacgtactctcaacagttcgcttaggcatggaagttttgcggcattctggctacacaataacaagggaagacttactcgtggctgcaaccctactagctcaaaatttattcacacatggttacgctttggggaaattatgataatgatccagatcacttctggctaataaaagatcagagctctagagatctgtgtgttggttttttgtggatctgctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcagcacagcaagggggaggattgggaagacaatagcaggcatgctggggatgcggtgggctctatgggtacctctctctctctctctctctctctctctctctctctctctcggtacctctctctctctctctctctctctctctctctctctctctcggtaccaggtgctgaagaattgacccggtgaccaaaggtgccttttatcatcactttaaaaataaaaaacaattactcagtgcctgttataagcagcaattaattatgattgatgcctacatcacaacaaaaactgatttaacaaatggttggtctgccttagaaagtatatttgaacattatcttgattatattattgataataataaaaaccttatccctatccaagaagtgatgcctatcattggttggaatgaacttgaaaaaaattagccttgaatacattactggtaaggtaaacgccattgtcagcaaattgatccaagagaaccaacttaaagctttcctgacggaatgttaattctcgttgaccctgagcactgatgaatcccctaatgattttggtaaaaatcattaagttaaggtggatacacatcttgtcatatgatcccggtaatgtgagttagctcactcattaggcaccccaggctttacactttatgcttccggctcgtatgttgtgtggaattgtgagcggataacaatttcacacaggaaacagctatgaccatgattacgccaagcgcgcaattaaccctcactaaagggaacaaaagctggagctccaccgcggtggcggccgctctagaactagtggatcccccgggctgcaggaattcgatatcaagcttatcgataccgctgacctcgagcatcagattggctattggccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgtcaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtcttcaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccgcggccgggaacggtgcattggaacgcggattccccgtgccaagagtgacgtaagtaccgcctatagactctataggcacacccctttggctcttatgcatgctatactgtttttggcttggggcctatacacccccgcttccttatgctataggtgatggtatagcttagcctataggtgtgggttattgaccattattgaccactcccctattggtgacgatactttccattactaatccataacatggctctttgccacaactatctctattggctatatgccaatactctgtccttcagagactgacacggactctgtatttttacaggatggggtcccatttattatttacaaattcacatatacaacaacgccgtcccccgtgcccgcagtttttattaaacatagcgtgggatctccacgcgaatctcgggtacgtgttccggacatgggctcttctccggtagcggcggagcttccacatccgagccctggtcccatgcctccagcggctcatggtcgctcggcagctccttgctcctaacagtggaggccagacttaggcacagcacaatgcccaccaccaccagtgtgccgcacaaggccgtggcggtagggtatgtgtctgaaaatgagcgtggagattgggctcgcacggctgacgcagatggaagacttaaggcagcggcagaagaagatgcaggcagctgagttgttgtattctgataagagtcagaggtaactcccgttgcggtgctgttaacggtggagggcagtgtagtctgagcagtactcgttgctgccgcgcgcgccaccagacataatagctgacagactaacagactgttcctttccatgggtcttttctgcagtcaccgtcggatcaatcattcatctcgtgacttcttcgtgtgtggtgtttacctatatatctaaatttaatatttcgtttattaaaatttaatatatttcgacgatgaatttctcaaggatatttttcttcgtgttcgctttggttctggctttgtcaacagtttcggctgcgccagagccgaaaggtacccaggtgcagctgcaggagtcggggggaggcttggtaaagccgggggggtcccttagagtctcctgtgcagcctctggattcactttcagaaacgcctggatgagctgggtccgccaggctccagggaaggggctggagtgggtcggccgtattaaaagcaaaattgatggtgggacaacagactatgctgcacccgtgaaaggcagattcaccatctcaagagatgattcaaaaaacacgttatatctgcaaatgaatagcctgaaagccgaggacacagccgtatattactgtaccacggggattatgataacatttgggggagttatccctcccccgaattggggccagggaaccctggtcaccgtctcctcagcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacacctttccggctgtcctacagtcctcaggactctacttccttagcaacgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaagcgccagagccgaagctttcctatgagctgacacagccaccctcggtgtcagtgtccccaggacaaacggccaggatcacctgctctggagatgcattgccagaaaaatatgtttattggtaccagcagaagtcaggccaggcccctgtggtggtcatctatgaggacagcaaacgaccctccgggatccctgagagattctctggctccagctcagggacaatggccaccttgactatcagtggggcccaggtggaagatgaaggtgactactactgttactcaactgacagcagtggttatcatagggaggtgttcagcggagggaccaagctgaccgtcctaggtcagcccaaggctgccccctcggtcactctgttcccaccctcctctgaggagcttcaagccaacaaggccacactggtgtgtctcataagtgactcctacccgggagccgtgacagtggcctggaaggcagatagcagccccgtcaaggcgggagtggagaccaccacaccctccaaacaaagcaacaacaagtacgcggccagcagctacctgagcctgacgcttgagcagtggaagtcccacaaaagctacagctgccaggtcacgcatgaagggagcaccgtggagaagacagtggcccctgcagaatgttcaccgcggagggagggaagggccctttttgaagggggaggaaacttcgcgccatgactcctctcgtgccccccgcacggaacactgatgtgcagagggccctctgccattgctgcttcctctgcccttcctcgtcactctgaatgtggcttctttgctactgccacagcaagaaataaaatctcaacatctaaatgggtttcctgagatttttcaagagtcgttaagcacattccttccccagcaccccttgctgcaggccagtgccaggcaccaacttggctactgctgcccatgagagaaatccagttcaatattttccaaagcaaaatggattacatatgccctagatcctgattaacaggtgttttgtattatctgtgctttcgcttcacccacattatcccattgcctcccctcgactcgagggggggcccggtacccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactactccgggatcatatgacaagatgtgtatccaccttaacttaatgatttttaccaaaatcattaggggattcatcagtgctcagggtcaacgagaattaacattccgtcaggaaagcttatgatgatgatgtgcttaaaaacttactcaatggctggttatgcatatcgcaatacatgcgaaaaacctaaaagagcttgccgataaaaaaggccaatttattgctatttaccgcggctttttattgagcttgaaagataaataaaatagataggttttatttgaagctaaatcttctttatcgtaaaaaatgccctcttgggttatcaagagggtcattatatttcgcggaataacatcatttggtgacgaaataactaagcacttgtctcctgtttactcccctgagcttgaggggttaacatgaaggtcatcgatagcaggataataatacagtaaaacgctaaaccaataatccaaatccagccatcccaaattggtagtgaatgattataaataacagcaaacagtaatgggccaataacaccggttgcattggtaaggctcaccaataatccctgtaaagcaccttgctgatgactctttgtttggatagacatcactccctgtaatgcaggtaaagcgatcccaccaccagccaataaaattaaaacagggaaaactaaccaaccttcagatataaacgctaaaaaggcaaatgcactactatctgcaataaatccgagcagtactgccgttttttcgcccatttagtggctattcttcctgccacaaaggcttggaatactgagtgtaaaagaccaagacccgtaatgaaaagccaaccatcatgctattcatcatcacgatttctgtaatagcaccacaccgtgctggattggctatcaatgcgctgaaataataatcaacaaatggcatcgttaaataagtgatgtataccgatcagcttttgttccctttagtgagggttaattgcgcgcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccac